(19) United States
(12) Patent Application Publication
(10) Pub. No.: US 2025/0262167 A1
(43) Pub. Date: Aug. 21, 2025

(54) COMPOUNDS, TARGETS AND PATHWAYS FOR MACROPHAGE MODULATION

(71) Applicant: Massachusetts Institute of Technology, Cambridge, MA (US)

(72) Inventors: Jianzhu Chen, Lexington, MA (US); Guangan Hu, Westwood, MA (US)

(21) Appl. No.: 19/035,427

(22) Filed: Jan. 23, 2025

Related U.S. Application Data
(62) Division of application No. 17/381,857, filed on Jul. 21, 2021, now abandoned.
(60) Provisional application No. 63/080,988, filed on Sep. 21, 2020.

(51) Int. Cl. A61K 31/03; G01N 33/50

(52) U.S. Cl. CPC A61K 31/03; G01N 33/5026

(57) ABSTRACT

Disclosed are methods of modulating macrophage activation to treat various diseases, such as cancer, fibrosis, infectious diseases, inflammatory diseases, metabolic diseases, or autoimmune diseases. Also disclosed are methods of identifying compounds useful for modulating macrophage activation as means to treat cancer, fibrosis, infectious diseases, inflammatory diseases, metabolic diseases, or autoimmune diseases.

### RELATED APPLICATIONS

This application is a division of application Ser. No. 17/381,857, filed Jul. 21, 2021; which claims the benefit of priority to U.S. Provisional Patent Application Ser. No. 63/080,988, filed Sep. 21, 2020; the contents of each of said applications are hereby incorporated herein by reference in their entirety.

### GOVERNMENT SUPPORT

This invention was made with Government support under Grant No. R35 CA197605 awarded by the National Institutes of Health (NIH). The Government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on Apr. 14, 2025, is named MTV-19202_SL.xml and is 36,310 bytes in size.

### BACKGROUND

Macrophages play an essential role in development, tissue homeostasis and repair, and immunity. Most macrophages exhibit multi-dimensional spectrum of phenotypes in response to various physiological and pathological signals. Because of their critical function in maintaining tissue homeostasis and repair, dysregulation of macrophage polarization has been implicated in contributing to many human diseases including cancer, fibrosis, obesity, diabetes, and infectious, cardiovascular, inflammatory and neurodegenerative diseases. Accordingly, there is a great need to identify modulators of macrophage activation for disease intervention.

### SUMMARY OF THE INVENTION

In one aspect, described herein is a method of identifying a modulator of macrophage activation. The method comprises contacting a primary macrophage cell with a candidate agent; monitoring or photographing the morphology of the cell contacted with the candidate agent; and optionally comparing the cell's morphology in the presence of the candidate agent with the cell's morphology in the absence of the candidate agent; wherein a change in morphology in the presence of the candidate agent is indicative of modulation of macrophage activation. Numerous embodiments are further provided that can be applied to any aspect of the present invention described herein. For example, in some embodiments, the primary macrophage cell is a bone marrow-derived macrophage or a monocyte-derived macrophage. In some embodiments, the morphology of the cell is monitored or photographed by a microscope, such as a fluorescence microscope. In some embodiments, the morphology of the cell is monitored or photographed by Opera Phenix high content screening system or CellProfiler. In some embodiments, the morphology of the cell is changed from elongated shape to round shape. In some embodiments, the modulator activates a M1-like macrophage, deactivates a M2-like macrophage, changes a tumor-associated macrophage (TAM) to M1-like macrophage, changes a M2-like macrophage to a M1-like macrophage, changes a M-CSF macrophage to a M1-like macrophage, changes a GM-CSF macrophage to a M1-like macrophage, changes a primary macrophage to a M1-like macrophage, induces LPS, IFNγ or TNFα, or activates a serotonin transporter or receptor, a histamine transporter or receptor, a dopamine transporter or receptor, an adrenoceptor, VEGF, EGF and/or leptin. In some embodiments, the modulator is a M1-activating compound. In some embodiments, the modulator is cytochalasin-B, fenbendazole, parbendazole, methiazole, alprostadil, FTY720, penfluridol, taxol, smer-3, cantharidin, SCH79797, mitoxantrone, niclosamide, MS275, HMN-214, DPI, thiostrepton, evodiamine, cucurbitacin-I, NVP 231, Chlorhexidine, Diphenyleneiodonium, LE135, Fluvoxamine, Mocetinostat, Pimozide, NP-010176, Celastrol, FTY720, WP1130, Prulifloxacin, dihydrocelastryl diacetate, or Quinolinium. In some embodiments, the M1-like macrophage mediates a pro-inflammatory response, an anti-microbial response, and/or an anti-tumor response. In some embodiments, the modulator treats cancer, fibrosis, and/or an infectious disease. In some embodiments, the cancer is hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, bladder cancer, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, or superficial spreading melanoma. In some embodiments, the infectious disease is a viral infection, or a bacterial infection. The infection may be associated with COVID-19 (SARS-CoV-2), SARS-CoV, MERS-CoV, Ebola virus, influenza, cytomegalovirus, variola and group A Streptococcus, or sepsis.

In some embodiments, the morphology of the cell is changed from round shape to elongated shape. In some embodiments, the modulator activates a M2-like macrophage, deactivates a M1-like macrophage, changes a M1-like macrophage to a M2-like macrophage, changes a M-CSF macrophage to a M2-like macrophage, changes a GM-CSF macrophage to a M2-like macrophage, changes a primary macrophage to a M2-like macrophage, modulator induces a M2-activating stimuli selected from IL4, IL13 and IL10, or inhibits a serotonin transporter or receptor, a histamine transporter or receptor, a dopamine transporter or receptor, an adrenoceptor, VEGF, EGF and/or leptin. In some embodiments, the modulator is a M2-activating compound. In some embodiments, the modulator is Bostunib, Su11274, Alsterpaullone, Alrestatin, Bisantrene, triptolide, lovastatin, QS 11, Regorafenib, Sorafenib, MLN2238, GW-843682X, KW 2449, Axitinib, JTE 013, Purmorphamine, Arcyriaflavin A, Dasatinib, NVP-LDE225, 1-Naphthyl PP1, Selamectin, MGCD-265, podofilox, colchicine, or vinblastine sulfate. In some embodiments, the M2-like macrophage mediates an anti-inflammatory or a tissue repair response. In some embodiments, the modulator treats an inflammatory disease, a metabolic disease, an autoimmune disease, or a neurodegenerative disease. In some embodiments, the inflammatory disease, the metabolic disease, or the autoimmune disease is diabetes, obesity, non-alcoholic fatty liver disease (NAFLD), hepatic steatosis, non-alcoholic steatohepatitis, cirrhosis, rheumatoid arthritis (RA), acute respiratory distress syndrome (ARDS), cardiovascular disease, remote tissue injury after ischemia and reperfusion, dermatomyositis, pemphigus, lupus nephritis and resultant glomerulonephritis and vasculitis, cardiopulmonary bypass, cardioplegia-induced coronary endothelial dysfunction, type II membranoproliferative glomerulonephritis, IgA nephropathy, acute renal failure, cryoglobulinemia, antiphospholipid syndrome, Chronic open-angle glaucoma, acute closed angle glaucoma, macular degenerative diseases, age-related macular degeneration (AMD), choroidal neovascularization (CNV), uveitis, diabetic retinopathy, ischemia-related retinopathy, endophthalmitis, intraocular neovascular disease, diabetic macular edema, pathological myopia, von Hippel-Lindau disease, histoplasmosis of the eye, Neuromyelitis Optica (NMO), Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, Anti-Neutrophilic Cytoplasmic Autoantibody vasculitis, Purtscher retinopathy, Sjogren's dry eye disease, dry AMD, sarcoidosis, temporal arteritis, polyarteritis nodosa, multiple sclerosis, hyperacute rejection, hemodialysis, chronic occlusive pulmonary distress syndrome (COPD), asthma, aspiration pneumonia, multiple sclerosis, Guillain-Barre syndrome, Myasthenia Gravis, Bullous Pemphigoid, or myositis. In some embodiments, the neurodegenerative disease is Alzheimer's disease, amyotrophic lateral sclerosis, multiple sclerosis, glaucoma, myotonic dystrophy, Guillain-Barre' syndrome (GBS), Myasthenia Gravis, Bullous Pemphigoid, spinal muscular atrophy, Down syndrome, Parkinson's disease, or Huntington's disease.

In one aspect, described herein is a method of treating cancer, fibrosis, or an infectious disease. The method comprises administering to a subject in need thereof an effective amount of a modulator of macrophage activation; wherein the modulator changes the morphology of a macrophage cell from elongated shape to round shape. Numerous embodiments are further provided that can be applied to any aspect of the present invention described herein. For example, in some embodiments, the modulator activates a M1-like macrophage, deactivates a M2-like macrophage, changes a tumor-associated macrophage (TAM) to M1-like macrophage, changes a M2-like macrophage to a M1-like macrophage, changes a M-CSF macrophage to a M1-like macrophage, changes a GM-CSF macrophage to a M1-like macrophage, changes a primary macrophage to a M1-like macrophage, induces a M1-activating stimuli selected from LPS, IFNγ and TNFα, or activates a serotonin transporter or receptor, a histamine transporter or receptor, a dopamine transporter or receptor, an adrenoceptor, VEGF, EGF and/or leptin. In some embodiments, the modulator is a M1-activating compound. In some embodiments, the modulator is cytochalasin-B, fenbendazole, parbendazole, methiazole, alprostadil, FTY720, penfluridol, taxol, smer-3, cantharidin, SCH79797, mitoxantrone, niclosamide, MS275, HMN-214, DPI, thiostrepton, evodiamine, cucurbitacin-I, NVP 231, Chlorhexidine, Diphenyleneiodonium, LE135, Fluvoxamine, Mocetinostat, Pimozide, NP-010176, Celastrol, FTY720, WP1130, Prulifloxacin, dihydrocelastryl diacetate, or Quinolinium. In some embodiments, the M1-like macrophage mediates a pro-inflammatory response, an anti-microbial response, and/or an anti-tumor response. In some embodiments, the cancer is hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, bladder cancer, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, or superficial spreading melanoma. In some embodiments, the method further comprises administering to the subject an effective amount of a second cancer therapy. In some embodiments, the second cancer therapy comprises cancer immunotherapy. In some embodiments, the cancer immunotherapy comprises administering an immune checkpoint inhibitor, such as an antibody or antigen-binding fragment thereof that specifically binds to an immune checkpoint protein. The immune checkpoint protein may be CTLA4, PD-1, PD-L1, PD-L2, A2AR, B7-H3, B7-H4, BTLA, KIR, LAG3, TIM-3 or VISTA. The immune checkpoint inhibitor may be atezolizumab, avelumab, durvalumab, ipilimumab, nivolumab, pembrolizumab, pidilizumab, AMP-224, AMP-514, BGB-A317, STI-A1110, TSR-042, RG-7446, BMS-936559, MEDI-4736, MSB-0020718C, AUR-012 or STI-A1010. In some embodiments, the second cancer therapy comprises the administration of a chemotherapy agent, such as rituxumab, thiotepa, cyclosphosphamide, busulfan, improsulfan, piposulfan, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, calicheamicin, dynemicin, clodronate, esperamicin; neocarzinostatin chromophore, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, epothilone, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK polysaccharide complex, razoxane, rhizoxin, sizofuran, spirogermanium, tenuazonic acid, triaziquone; 2,2′,2″-trichlorotriethylamine, trichothecene, T-2 toxin, verracurin A, roridin A, anguidine, urethane, vindesine, dacarbazine, mannomustine, mitobronitol, mitolactol, pipobroman, gacytosine, arabinoside, cyclophosphamide, thiotepa, paclitaxel, doxetaxel, chlorambucil, gemcitabine, 6-thioguanine, mercaptopurine, methotrexate, cisplatin, oxaliplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, irinotecan, RFS 2000, difluoromethylomithine, retinoic acid or capecitabine. In some embodiments, the infectious disease is a viral infection, or a bacterial infection. In some embodiments, the infection is associated with COVID-19 (SARS-CoV-2), SARS-CoV, MERS-CoV, Ebola virus, influenza, cytomegalovirus, variola and group A Streptococcus, or sepsis.

In one aspect, described herein is a method of treating an inflammatory disease, a metabolic disease, an autoimmune disease, or a neurodegenerative disease. The method comprises administering to a subject in need thereof an effective amount of a modulator of macrophage activation; wherein the modulator changes the morphology of a macrophage cell from round shape to elongated shape. Numerous embodiments are further provided that can be applied to any aspect of the present invention described herein. For example, in some embodiments, the modulator activates a M2-like macrophage, deactivates a M1-like macrophage, changes a M1-like macrophage to a M2-like macrophage, changes a M-CSF macrophage to a M2-like macrophage, changes a GM-CSF macrophage to a M2-like macrophage, changes a primary macrophage to a M2-like macrophage, induces a M2-activating stimuli selected from IL4, IL13 and IL10, or inhibits a serotonin transporter or receptor, a histamine transporter or receptor, a dopamine transporter or receptor, an adrenoceptor, VEGF, EGF and/or leptin. In some embodiments, the modulator is a M2-activating compound. In some embodiments, the modulator is Bostunib, Su11274, Alsterpaullone, Alrestatin, Bisantrene, triptolide, lovastatin, QS 11, Regorafenib, Sorafenib, MLN2238, GW-843682X, KW 2449, Axitinib, JTE 013, Purmorphamine, Arcyriaflavin A, Dasatinib, NVP-LDE225, 1-Naphthyl PP1, Selamectin, MGCD-265, podofilox, colchicine, or vinblastine sulfate. In some embodiments, the M2-like macrophage mediates an anti-inflammatory or a tissue repair response. In some embodiments, the inflammatory disease, the metabolic disease, or the autoimmune disease is diabetes, obesity, non-alcoholic fatty liver disease (NAFLD), hepatic steatosis, non-alcoholic steatohepatitis, cirrhosis, rheumatoid arthritis (RA), acute respiratory distress syndrome (ARDS), cardiovascular disease, remote tissue injury after ischemia and reperfusion, dermatomyositis, pemphigus, lupus nephritis and resultant glomerulonephritis and vasculitis, cardiopulmonary bypass, cardioplegia-induced coronary endothelial dysfunction, type II membranoproliferative glomerulonephritis, IgA nephropathy, acute renal failure, cryoglobulinemia, antiphospholipid syndrome, Chronic open-angle glaucoma, acute closed angle glaucoma, macular degenerative diseases, age-related macular degeneration (AMD), choroidal neovascularization (CNV), uveitis, diabetic retinopathy, ischemia-related retinopathy, endophthalmitis, intraocular neovascular disease, diabetic macular edema, pathological myopia, von Hippel-Lindau disease, histoplasmosis of the eye, Neuromyelitis Optica (NMO), Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, Anti-Neutrophilic Cytoplasmic Autoantibody vasculitis, Purtscher retinopathy, Sjogren's dry eye disease, dry AMD, sarcoidosis, temporal arteritis, polyarteritis nodosa, multiple sclerosis, hyperacute rejection, hemodialysis, chronic occlusive pulmonary distress syndrome (COPD), asthma, aspiration pneumonia, multiple sclerosis, Guillain-Barre syndrome, Myasthenia Gravis, Bullous Pemphigoid, or myositis. In some embodiments, the neurodegenerative disease is Alzheimer's disease, amyotrophic lateral sclerosis, multiple sclerosis, glaucoma, myotonic dystrophy, Guillain-Barre' syndrome (GBS), Myasthenia Gravis, Bullous Pemphigoid, spinal muscular atrophy, Down syndrome, Parkinson's disease, or Huntington's disease.

In one aspect, described herein is a method of treating cancer, fibrosis, or an infectious disease. The method comprises administering to a subject in need thereof an effective amount of a modulator of macrophage activation; wherein the modulator activates a serotonin transporter or receptor, a histamine transporter or receptor, a dopamine transporter or receptor, an adrenoceptor, VEGF, EGF and/or leptin. Numerous embodiments are further provided that can be applied to any aspect of the present invention described herein. For example, in some embodiments, the modulator is cytochalasin-B, fenbendazole, parbendazole, methiazole, alprostadil, FTY720, penfluridol, taxol, smer-3, cantharidin, SCH79797, mitoxantrone, niclosamide, MS275, HMN-214, DPI, thiostrepton, evodiamine, cucurbitacin-I, NVP 231, Chlorhexidine, Diphenyleneiodonium, LE135, Fluvoxamine, Mocetinostat, Pimozide, NP-010176, Celastrol, FTY720, WP1130, Prulifloxacin, dihydrocelastryl diacetate, or Quinolinium. In some embodiments, the cancer is hematological malignancy, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, telangiectaltic sarcoma, Hodgkin's Disease, Non-Hodgkin's Lymphoma, multiple myeloma, neuroblastoma, bladder cancer, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, or superficial spreading melanoma. In some embodiments, the method further comprises administering to the subject an effective amount of a second cancer therapy. In some embodiments, the second cancer therapy is cancer immunotherapy, such as an immune checkpoint inhibitor, for example, an antibody or antigen-binding fragment thereof that specifically binds to an immune checkpoint protein. In some embodiments, the immune checkpoint protein is CTLA4, PD-1, PD-L1, PD-L2, A2AR, B7-H3, B7-H4, BTLA, KIR, LAG3, TIM-3 or VISTA. In some embodiments, the immune checkpoint inhibitor is atezolizumab, avelumab, durvalumab, ipilimumab, nivolumab, pembrolizumab, pidilizumab, AMP-224, AMP-514, BGB-A317, STI-A1110, TSR-042, RG-7446, BMS-936559, MEDI-4736, MSB-0020718C, AUR-012 or STI-A1010. In some embodiments, the second cancer therapy is a chemotherapy agent, such as rituxumab, thiotepa, cyclosphosphamide, busulfan, improsulfan, piposulfan, benzodopa, carboquone, meturedopa, uredopa, altretamine, triethylenemelamine, trietylenephosphoramide, triethiylenethiophosphoramide, trimethylolomelamine, bullatacin, bullatacinone, camptothecin, topotecan, bryostatin, callystatin, CC-1065, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimnustine, calicheamicin, dynemicin, clodronate, esperamicin; neocarzinostatin chromophore, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycin, mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, zorubicin, methotrexate, 5-fluorouracil (5-FU), denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elformithine, elliptinium acetate, epothilone, etoglucid, gallium nitrate, hydroxyurea, lentinan, lonidainine, maytansine, ansamitocins, mitoguazone, mitoxantrone, mopidanmol, nitraerine, pentostatin, phenamet, pirarubicin, losoxantrone, podophyllinic acid, 2-ethylhydrazide, procarbazine, PSK polysaccharide complex, razoxane, rhizoxin, sizofuran, spirogermanium, tenuazonic acid, triaziquone; 2,2′,2″-trichlorotriethylamine, trichothecene, T-2 toxin, verracurin A, roridin A, anguidine, urethane, vindesine, dacarbazine, mannomustine, mitobronitol, mitolactol, pipobroman, gacytosine, arabinoside, cyclophosphamide, thiotepa, paclitaxel, doxetaxel, chlorambucil, gemcitabine, 6-thioguanine, mercaptopurine, methotrexate, cisplatin, oxaliplatin, carboplatin, vinblastine, platinum, etoposide, ifosfamide, mitoxantrone, vincristine, vinorelbine, novantrone, teniposide, edatrexate, daunomycin, aminopterin, xeloda, ibandronate, irinotecan, RFS 2000, difluoromethylomithine, retinoic acid or capecitabine. In some embodiments, the infectious disease is a viral infection, or a bacterial infection. In some embodiments, the infection is associated with COVID-19 (SARS-CoV-2), SARS-CoV, MERS-CoV, Ebola virus, influenza, cytomegalovirus, variola and group A Streptococcus, or sepsis.

In one aspect, described herein is a method of treating an inflammatory disease, a metabolic disease, an autoimmune disease, or a neurodegenerative disease. The method comprises administering to a subject in need thereof an effective amount of a modulator of macrophage activation; wherein the modulator inhibits a serotonin transporter or receptor, a histamine transporter or receptor, a dopamine transporter or receptor, an adrenoceptor, VEGF, EGF and/or leptin. Numerous embodiments are further provided that can be applied to any aspect of the present invention described herein. For example, in some embodiments, the modulator is Bostunib, Su11274, Alsterpaullone, Alrestatin, Bisantrene, triptolide, lovastatin, QS 11, Regorafenib, Sorafenib, MLN2238, GW-843682X, KW 2449, Axitinib, JTE 013, Purmorphamine, Arcyriaflavin A, Dasatinib, NVP-LDE225, 1-Naphthyl PP1, Selamectin, MGCD-265, podofilox, colchicine, or vinblastine sulfate. In some embodiments, the inflammatory disease, the metabolic disease, or the autoimmune disease is diabetes, obesity, non-alcoholic fatty liver disease (NAFLD), hepatic steatosis, non-alcoholic steatohepatitis, cirrhosis, rheumatoid arthritis (RA), acute respiratory distress syndrome (ARDS), cardiovascular disease, remote tissue injury after ischemia and reperfusion, dermatomyositis, pemphigus, lupus nephritis and resultant glomerulonephritis and vasculitis, cardiopulmonary bypass, cardioplegia-induced coronary endothelial dysfunction, type II membranoproliferative glomerulonephritis, IgA nephropathy, acute renal failure, cryoglobulinemia, antiphospholipid syndrome, Chronic open-angle glaucoma, acute closed angle glaucoma, macular degenerative diseases, age-related macular degeneration (AMD), choroidal neovascularization (CNV), uveitis, diabetic retinopathy, ischemia-related retinopathy, endophthalmitis, intraocular neovascular disease, diabetic macular edema, pathological myopia, von Hippel-Lindau disease, histoplasmosis of the eye, Neuromyelitis Optica (NMO), Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, Anti-Neutrophilic Cytoplasmic Autoantibody vasculitis, Purtscher retinopathy, Sjogren's dry eye disease, dry AMD, sarcoidosis, temporal arteritis, polyarteritis nodosa, multiple sclerosis, hyperacute rejection, hemodialysis, chronic occlusive pulmonary distress syndrome (COPD), asthma, aspiration pneumonia, multiple sclerosis, Guillain-Barre syndrome, Myasthenia Gravis, Bullous Pemphigoid, or myositis. In some embodiments, the neurodegenerative disease is Alzheimer's disease, amyotrophic lateral sclerosis, multiple sclerosis, glaucoma, myotonic dystrophy, Guillain-Barre' syndrome (GBS), Myasthenia Gravis, Bullous Pemphigoid, spinal muscular atrophy, Down syndrome, Parkinson's disease, or Huntington's disease.

In one aspect, described herein is a method of treating an inflammatory disease, a metabolic disease, an autoimmune disease, or a neurodegenerative disease. The method comprises administering to a subject in need thereof an effective amount of diphenyleneiodonium (DPI). Numerous embodiments are further provided that can be applied to any aspect of the present invention described herein. For example, in some embodiments, the inflammatory disease, the metabolic disease, or the autoimmune disease is diabetes, obesity, Non-alcoholic fatty liver disease (NAFLD), hepatic steatosis, non-alcoholic steatohepatitis, cirrhosis, rheumatoid arthritis (RA), acute respiratory distress syndrome (ARDS), cardiovascular disease, remote tissue injury after ischemia and reperfusion, dermatomyositis, pemphigus, lupus nephritis and resultant glomerulonephritis and vasculitis, cardiopulmonary bypass, cardioplegia-induced coronary endothelial dysfunction, type II membranoproliferative glomerulonephritis, IgA nephropathy, acute renal failure, cryoglobulinemia, antiphospholipid syndrome, Chronic open-angle glaucoma, acute closed angle glaucoma, macular degenerative diseases, age-related macular degeneration (AMD), choroidal neovascularization (CNV), uveitis, diabetic retinopathy, ischemia-related retinopathy, endophthalmitis, intraocular neovascular disease, diabetic macular edema, pathological myopia, von Hippel-Lindau disease, histoplasmosis of the eye, Neuromyelitis Optica (NMO), Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, Anti-Neutrophilic Cytoplasmic Autoantibody vasculitis, Purtscher retinopathy, Sjogren's dry eye disease, dry AMD, sarcoidosis, temporal arteritis, polyarteritis nodosa, multiple sclerosis, hyperacute rejection, hemodialysis, chronic occlusive pulmonary distress syndrome (COPD), asthma, aspiration pneumonia, multiple sclerosis, Guillain-Barre syndrome, Myasthenia Gravis, Bullous Pemphigoid, or myositis. In some embodiments, the neurodegenerative disease is Alzheimer's disease, amyotrophic lateral sclerosis, multiple sclerosis, glaucoma, myotonic dystrophy, Guillain-Barre' syndrome (GBS), Myasthenia Gravis, Bullous Pemphigoid, spinal muscular atrophy, Down syndrome, Parkinson's disease, or Huntington's disease.

### BRIEF DESCRIPTION OF THE DRAWINGS

FIGS. 1A-1H show a high throughput screen for compounds that activate human macrophages. FIG. 1A and FIG. 1B show that hMDMs were cultured for 24 hours in the presence of LPS, IFNγ, TNFα, IFNγ plus TNFα (I+T), IL-10, IL-4 or IL-13. Shown are examples of cell morphologies of M1-activating macrophages by IFNγ and M2-activating macrophages by IL-4 (FIG. 1A) and calculated Z-scores for each stimulus (FIG. 1B) from three independent experiments. Each symbol represents a technical replicate. The Z-score was calculated by T-test to measure the difference of cell morphology between treatment and control. Stimuli had negative Z-scores when induced cells to round morphology and positive scores when induced cells to elongated morphology. FIG. 1C shows the flowchart of screening and data analysis. Equally mixed human monocytes isolated from fresh blood of 4 healthy donors were cultured in vitro with 50 ng/mL M-CSF for 7 days. hMDMs were trypsinized and plated on 384-well plates (5000 cells/well in 50 μL). Cells were recovered in 10 ng/mL M-CSF for 16 hrs and then treated with compounds for 24 hrs. Cells were washed, fixed and stained with Phalloidin and DAPI. The plates were scanned with a high-content microscope with six-fields per well to quantify the cell number and cell morphology. FIG. 1D shows composition of compound libraries used in the screen. FIG. 1E shows examples of cell shape changes induced by two compounds and their corresponding Z-scores as compared to DMSO controls. The cell eccentricity was calculated to measure the cell morphology. The Z-score was calculated by T-test to measure the difference in cell morphologies between each compound and DMSO control. FIG. 1F shows plot of Z-scores of 4126 compounds and number of cells captured in each well. The dash lines are the cutoffs for M1 activation (left) and M2 activation (right) based on the average of Z-scores from FIG. 1B. FIG. 1G shows classification of identified compounds based on their origination and the function of their known targets. FIG. 1H shows pathway analysis of known targets of identified M1- or M2-activating compounds. Each dot is one specific pathway having protein targets by compounds and dot size refer to the number of compounds. The average Z-score (y-axis) and number of compounds that have protein targets belongs to one specific pathway are plotted. Selected known (black) and novel (gray) pathways associated with macrophage activation are indicated.

FIGS. 2A-2F show validation of macrophage activation induced by compounds or by ligands of the identified novel pathways. FIGS. 2A-2B show that the morphology changes induced by selected compounds are dosage-dependent. Dosage response was calculated based on the measurement of Z-scores at different concentrations of the compound in a Michaelis-Menten model. Shown are representative dosage response curves of M1-activating (thiostrepton) and M2-activating (bosutinib) compounds (FIG. 2A). 25 of the 30 tested compounds had typical dosage dependent response (FIG. 2B). Effective concentration (EC) was defined as the concentration of compounds inducing cell morphology changes to reach the cutoffs of either M1 or M2. EC, fitness (R square) and Max Z-score were calculated by the Michaelis-Menten equation. Data were summarized from 3 independent experiments. FIG. 2C shows GSEA of transcriptional response to 8 selected compounds and controls (IL-4 and IFNγ). Duplicate hMDM samples were treated with 2 M2-activating and 6 M1-activating compounds as well as IL-4 and IFNγ for 24 hrs. Gene expression levels were measured by RNA-seq separately. GSEA preranked analysis was performed based on the whole genome gene list ranked on gene expression changes using a gene set of 49 transcriptional modules in response to 29 stimuli in hMDMs. bosut.: bosutinib; alster.: alsterpaullone; mocet.: mocetinostat; thios.: thiostrepton; niclo.: niclosamide; chlor.: chlorhexidine; fenb.: fenbendazole; fluvo.: fluvoxamine. FIG. 2D shows GO enrichment analysis of DEGs induced by each compound and positive controls. The numbers of DEGs that are up and down regulated are indicated. FIG. 2E shows GSEA of transcriptional response to 6 ligands of the identified novel pathways in FIG. 1H. dopa.: dopamine; 5HT: serotonin. Duplicate hMDM samples were stimulated with each ligand and analyzed by RNA-seq separately. FIG. 2F shows GO enrichment analysis of DEGs induced by the ligands and positive controls. The numbers of DEGs that are up and down regulated are indicated.

FIGS. 3A-3E show reprogramming screen of compounds on differentiated macrophages. FIG. 3A shows that hMDMs were differentiated into M2 by IL4 plus IL13 and then treated with each of the 127 identified M1-activating compounds at either 5 μM or 10 μM for 24 hrs in the absence of differentiating cytokines. Shown are comparison of Z-scores between 5 μM or 10 μM of compounds. FIG. 3B shows that hMDMs were differentiated into M1 by IFNγ plus TNFα and then treated with each of the 180 identified M2-activating compounds at either 5 μM or 10 μM for 24 hrs in the absence of differentiating cytokines. Shown are comparison of Z-scores between 5 μM or 10 μM of compounds. FIG. 3C shows the effective concentration of 40 selected M1- or M2-activating compounds calculated from the dosage assays. EC and fitness of 21 M1-polarizing (triangle) and 19 M2-polarizing compounds (circle) were calculated by the Michaelis-Menten equation and plotted. Data were summarized from 3 independent experiments. FIGS. 3D-3E show that hMDMs were differentiated into either M2 by IL4 plus IL13 or M1 by IFNγ plus TNFα and then treated with 127 M1-activating (FIG. 3D) or 180 M2-activating (FIG. 3E) compounds for 24 hrs in the presence of differentiating cytokines. Filled dots showed the overlapping ones with the 37 M1-activating (FIG. 3A) and 21 M2-activating (FIG. 3B) compounds.

FIGS. 4A-4F show reprogramming of differentiated macrophages by selected compounds. FIG. 4A shows number of DEGs induced by each compound: upregulated genes and down-regulated genes. hMDMs were differentiated into either M2 by IL-4 plus IL-13 or M1 by IFNγ plus TNFα and duplicate samples were then treated with either M1-activating or M2-activating compounds, respectively, at the effective concentrations. Controls include two differentiated M1 and M2 macrophages, M2 macrophages treated with IFNγ and M1 macrophages treated with IL-4. Gene expression in each sample was measured by RNA-seq separately. FIG. 4B shows hierarchical clustering heatmap of Pearson correlation coefficients for 7620 DEGs induced by compounds as well as IFNγ and IL-4. FIG. 4C shows GSEA analysis of transcriptional responses to each compound as compared to IFNγ and IL-4. FIG. 4D show network of GO enriched terms using BiNGO on top 10% central hubs genes (n=1255) of macrophage activation network. Node color and size represent the FDR values of enriched GO terms. FIGS. 4E-4F shows functional enrichment analysis of DEGs induced by each compound. Shared (FIG. 4E) and unique pathways (FIG. 4F) are shown. Compound targets and FDA-approval information are indicated. The order of M1-activating and M2-activating compounds in FIG. 4E and FIG. 4F are the same as in FIG. 4A.

FIGS. 5A-5E show that thiostrepton induces macrophages into pro-inflammatory state and enhances anti-tumor activity in vitro. FIG. 5A shows volcano plot showing changes in transcription in hMDMs induced by thiostrepton (n=2). hMDMs were treated with 2.5 μM thiostrepton for 24 hrs followed by RNA-seq. DEGs were identified by edgeR at P<0.05 with at least 2 fold-change. Data for genes that were not classified as differentially expressed are plotted in black. Filled dots represent upregulated and down-regulated genes as shown. FIG. 5B shows GO enrichment analysis of DEGs induced by thiostrepton. FIG. 5C shows GSEA of transcriptional response to thiostrepton. FIG. 5D shows that thiostrepton inhibits the development and function of TAMs in vitro. Mouse BMMs were cultured in normal medium with or without 2.5 μM thiostrepton for 24 hrs (group 1), or cultured in B16F10 tumor cell conditioned medium (CM) with or without 2.5 μM thiostrepton for 24 hrs (group 2), or cultured with B16F10 tumor cell CM for 24 hrs first and then treated with 2.5 μM thiostrepton for another 24 hrs (group 3). The transcript levels of the indicated genes were quantified by qPCR. Data were summarized from two independent experiments. FIG. 5E shows that thiostrepton enhances anti-tumor activities of macrophages. Mouse BMMs were treated with thiostrepton for 24 hrs. Untreated and treated macrophages were co-cultured with equal number of B16F10 melanoma cells for 12 hrs. The number of tumor cells were quantified by flow cytometry after subtracting macrophages from total number of cells. Data were summarized from three independent experiments. ** P<0.01 by T-test.

FIGS. 6A-6F show that thiostrepton exhibits anti-tumor activities through reprogramming tumor-associated macrophages in vivo. FIG. 6A shows tumor growth curves in B6 mice bearing subcutaneous B16F10 tumors treated I.P. with DMSO, TA99, thiostrepton (300 mg/kg or 150 mg/kg) and thipstrepton plus TA99. Arrows indicate dosing time points. FIG. 6B shows tumor growth curves in B6 mice bearing subcutaneous B16F10 tumors treated I.P. with TA99, and S.C. with PBS or DMSO or thiostrepton (20 mg/kg) or thiostrepton plus TA99 (n=10-12 mice per group). FIGS. 6C-6D show flow cytometry analysis of TAM (F4/80⁺CD11b⁺Ly6C⁻Ly6G⁻), inflammatory monocytes (F4/80^{int}CD11b⁺Ly6C⁺Ly6G⁻) and monocytes (F4/80⁻CD11b⁺Ly6C⁺Ly6G⁺) in the tumors of control, TA99-treated, thiostrepton-treated and thiostrepton plus TA99-treated tumor-bearing mice 18 days after tumor engraftment. Shown are representative F4/80 versus CD11b staining profiles gating on CD45+ cells (FIG. 6C) and summarized data (FIG. 6D) from three independent experiments with 3-4 mice per group per experiment. Error bars indicate standard deviation (SD). FIG. 6E shows immunohistochemistry staining of F4/80 in tumor sections. Scale bar: 100 m. FIG. 6F shows comparison of gene expression changes induced by thiostrepton in tumor infiltrated macrophages by I.P. (n=4) or S.C. administration (n=2) of thiostrepton or DMSO (n=2). Tumor infiltrated macrophages were sorted from tumor issues based on CD45⁺F4/80⁺CD11b⁺ Gr-1⁻ 18 days after tumor engraftment. I.P.: intraperitoneal injection; S.C.: paratumor subcutaneous injection. * P<0.05 and ** P<0.01 by T-test.

FIGS. 7A-7B show morphology and phenotypes of activated macrophages. FIG. 7A shows F-actin staining of M1- and M2-like macrophages. hMDMs were induced to become M0 by M-CSF. The resulting macrophages were polarized to M1 by IFNγ or M2 by IL4. Then, M1 macrophages were treated with M2-type compound bosutinib (1 mM) for 24 hrs, and M2 macrophages were treated with M1-type compound thiostrepton (2.5 mM) for 24 hrs. F-actin was stained and images were acquired by fluorescent microscopy with 60× objective. Cell nuclei are stained with DAPI. Representative data were shown from two independent experiments. FIG. 7B shows CD163, CD206, CD80 and CD86 in hMDM treated with DMSO, or IFNγ or IL4 quantified by flow cytometry. Shown are the representative staining profiles from three independent experiments. The numbers show mean fluorescent intensity (MFI)+/−standard error of the mean (SEM) for n=3 samples per group.

FIG. 8 shows the top list of proteins that are targeted by M1-activating and M2-activating compounds. Histone deacetylases and VEGF receptors are highlighted gray.

FIGS. 9A-9C show comparison of the differentially expressed genes induced by selected compounds (FIG. 9A), ligands for novel pathways (FIG. 9B), and controls (IL-4 and IFNγ). FIG. 9C shows changes of the selected M1 markers (CD80 and CD86) and M2 markers (CD206 and CD163) at protein level induced by compound as assayed by flow cytometry. Shown are the changes of the relative mean fluorescence intensity (MFI) to controls. 0.2 refers to 20% MFI increase.

FIG. 10 shows comparison of EC of 21 M1-activating and 19 M2-activating compounds in the presence or absence of the polarizing cytokines.

FIGS. 11A-11E show reprogramming of differentiated macrophages by selected compounds. FIG. 11A shows principal component analysis of global transcriptional response of hMDMs to 17 M1-activating and 17 M2-activating compounds. The samples are the same as those in FIG. 4A. FIG. 11B shows functional enrichment analysis of DEGs induced by each compound. Shown is the assembled heatmap and number of up-regulated and down-regulated DEGs (bottom panel). FIG. 11C shows comparison of relative transcript levels of the selected M1 and M2 genes following compound treatment based on RNA-seq. FIG. 11D shows comparison of the transcript levels of the selected M1 and M2 genes following compound treatment as measured by quantitative PCR. FIG. 11E shows comparison of the protein levels of the selected M1 and M2 markers following compound treatment as measured by flow cytometry. Shown are the relative MFI change to controls. 0.2 refers to 20% MFI increase. The order of M1-activating and M2-activating compounds in b-e is the same as in FIG. 4A.

FIG. 12 shows macrophage activation network. The network was inferred by ARACNe (Margolin et al. 2006). The top 10% central hub gene network was visualized by Cytoscape (Shannon et al. 2003). The dark marked nodes are transcription factors (regulators). Top 10 central hubs and top 10 central TF hubs are listed.

FIGS. 13A-13B show that thiostrepton inhibits the development and function of M2-like macrophages in vitro. FIG. 13A shows mouse BMMs were cultured with B16F10 tumor cell conditioned medium (CM) for 24 hrs first and then treated with 2.5 mM thiostrepton for another 24 hrs (group 3 from FIG. 5D). Expression of MHCII, CD80, iNOS, Arg1 and CD206 were quantified by flow cytometry. Shown are representative staining profiles of treated (red) and untreated (dark) TAMs from two independent experiments. FIG. 13B shows that mouse BMMs were not treated or treated with 2.5 mM thiostrepton for 24 hrs in normal medium (group 1), or polarized with IL-4/IL-13 in the absence or presence of 2.5 mM thiostrepton for 24 hrs (group 2), or polarized with lactic acid in the absence or presence of 2.5 mM thiostrepton for 24 hrs (group 4). Alternatively, mouse BMMs were polarized with IL-4/IL-13 (group 3) or lactic acid (group 5) for 24 hrs first and then either not treated or treated with 2.5 mM thiostrepton for another 24 hrs. The transcript levels of the indicated genes were quantified by qPCR. Data are summarized from two independent experiments.

FIGS. 14A-14C show that thiostrepton activates macrophages in vitro. FIG. 14A shows that mouse BMMs were treated with thiostrepton for 24 hrs (same as FIG. 5E). Conditioned medium (CM) was collected and filtered. B16F10 melanoma cells were cultured for 12 hrs with CM or CM heat-inactivated at 95° C. for 5 min. The number of tumor cells were quantified by flow cytometry. Data were summarized from two independent experiments. * P<0.05 by T-test. P values are shown based on t-test. FIGS. 14B-14C show that thiostreption enhances ADCP of macrophages. Mouse BMMs (FIG. 14B) or hMDM (FIG. 14C) were treated with 2.5 mM thiostrepton for 24 hrs, then co-cultured with equal number of eFluro670 and anti-CD20 labelled human B-cell lymphoma cells for 2 hrs, and analyzed by flow cytometry. Macrophages that have phagocytosed tumor cells are identified as efluro670+ and CD14+. Shown are representative eFluro670 histograms gating on CD14+ macrophages from three different experiments.

FIGS. 15A-15B show that thiostrepton activates macrophages in vivo without altering the total number of gut bacterial counts. FIG. 15A shows flow cytometry analysis of macrophages (F4/80+CD11b+) and monocytes (F4/80−CD11b+) in the bone marrow and spleen of mice 6 days post treatment with either DMSO or thiostrepton by I.P. or S.C. (n=3). Shown are representative F4/80 versus CD11b staining profiles gating on CD45+ cells. I.P.: intraperitoneal injection; S.C.: paratumor subcutaneous injection. FIG. 15B shows total bacterial counts in the stool sample of mice. Data shown are mean±s.d. n.s., not significant by T-test.

FIGS. 16A-16D show effect of thiostrepton on macrophages, NK cells and CD8+ T cells in vivo. B6 mice bearing subcutaneous B16F10 tumor were treated as in FIG. 6. Single cell suspensions were prepared from tumors 18 day after engraftment, stained and analyzed by flow cytometry. FIGS. 16A-16B show representative intracellular staining profiles of Arg1 vs. CD86 gated on F4/80+CD11b+ Gr1− TAMs (FIG. 16A) and summarized data from n=5 mice per group from two independent experiments (FIG. 16B). FIG. 16C shows representative intracellular staining profiles of IFNγ vs. TNFα gated on CD45+ NK1.1+ NK cells (top two rows) and CD45+ CD8a+ T cells (bottom two rows). Samples for T-cell staining were stimulated in vitro by T-cell stimulation cocktail for 4 hrs. FIG. 16D shows summarized data from n=4˜6 mice per group from two independent experiments. * P<0.05 by T-test. Data shown are mean±s.d.

FIGS. 17A-17D show transcriptional response of TAMs to thiostrepton in vivo. FIG. 17A shows GO enrichment analysis showing enrichment of certain pathways in the up-regulated and down-regulated genes in TAMs following I.P. administration of thiostrepton or DMSO. GO sets of biological process, number of genes and P-value are shown. Tumor infiltrated macrophages were sorted from tumor tissues based on CD45+F4/80+CD11b+Gr1− 18 days after tumor engraftment. Gene expression levels were measured by RNAseq. FIG. 17B shows GSEA showing enriched gene sets in TAMs induced by thiostrepton in vivo by I.P. administration (FDR q-value <0.05). FIG. 17C shows GO enrichment analysis showing enrichment of certain pathways in the up-regulated and down-regulated genes in TAMs induced by S.C. administration of thiostrepton or DMSO. GO sets of biological process, number of genes and P-value are shown. FIG. 17D shows GSEA showing enriched gene sets in TAMs induced by thiostrepton in vivo by S.C. administration (FDR q-value <0.05). I.P.: intraperitoneal injection; S.C.: paratumor subcutaneous injection.

FIGS. 18A-18D show that thiostrepton inhibits tumor growth in the bone marrow. NSG mice were grafted with 1×10⁷ GMB-luc cells and dosed twice at 14 and 21 days later with 0.5 mg/kg Rituximab (Ritu) and/or 300 mg/kg thiostrepton (Thio). Tumor burden was monitored (FIG. 18A) and quantified (FIG. 18B) by imaging the luciferase activity in vivo (n=5˜6 mice per group). Data are shown as mean±s.e.m. At day 28 post tumor engraftment, bone marrow cells were analyzed by flow cytometry (FIG. 18C). Shown are representative F4/80 versus CD11b staining profiles gating on CD45⁺ cells (top panel), Ly6C versus Ly6G staining profiles gating on F4/80⁺CD11b⁺ cells (bottom panel). MHCII histograms gating on macrophages from FIG. 18C. FIG. 18D shows summarized data of MHCII expression in bone marrow macrophages from FIG. 18C. Data shown are mean±s.d. * P<0.05, ** P<0.01 and *** P<0.001, by T-test.

FIGS. 19A-19D shows that M1-type compound, cucurbitacin I, also activates macrophages and inhibits tumor growth. FIG. 19A shows that cucurbitacin I inhibits the development and function of tumor-associated macrophages in vitro induced by IL4/IL13. Mouse BMMs were not treated or treated with 2.5 mM thiostrepton for 24 hours in normal medium (group 1) or in the presence of IL4/IL13 (group 2), or mouse BMMs were polarized with IL4/IL13 for 24 hours and then either not treated or treated with 2.5 mM thiostrepton for 24 hours (group 3). RNA was isolated and the transcript levels of the indicated genes were quantified by PCR. Data, shown as mean±s.d., were summarized from two independent experiments. * P<0.05 and ** P<0.01 by T-test. FIG. 19B shows B16F10 tumor growth in B6 mice treated i.p. with DMSO, TA99, cucurbitacin I (1 mg/kg) and cucurbitacin I plus TA99 (n=6 mice per group). Data are shown as mean±s.e.m. FIGS. 19C-19D show flow cytometry analysis of TAM (F4/80⁺CD11b⁺Ly6C⁻Ly6G⁻), inflammatory monocytes (F4/80′^{int}CD11b⁺Ly6C⁺Ly6G⁻) and monocytes (F4/80⁻CD11b⁺Ly6C⁺Ly6G⁺) in the tumors of mice treated with DMSO, TA99, cucurbitacin I, and cucurbitacin I plus TA99 18 days after tumor engraftment. Shown are representative F4/80 versus CD11b staining profiles gating on CD45+ cells and MHCII histograms gating on macrophages from c.

FIGS. 20A-20F show that DPI stimulates both rapid and sustained increase in glycolysis in macrophages. FIG. 20A shows glycolysis pathway with involved enzymes and intermediates and TCA cycle with selected intermediates. FIGS. 20B-20C show the short-term effects of DPI on ECAR (FIG. 20B) and OCR (FIG. 20C) in ImKCs. ECAR and OCR were measured by Seahorse analyzer in ImKCs for 20 min, then for another 120 min following addition of different concentrations of DPI (5, 50 or 500 nM), and then for another 40 min following addition of rotenone plus antimycin A (Rot/AA) (FIG. 20B) or 2-deoxylglucose (2-DG) (FIG. 20C). Shown are representative data of three independent experiments. FIGS. 20D-20E show the long-term effects of DPI on ECAR. ImKCs were seeded and incubated with or without DPI (50 and 500 nM) for 24 hrs. ECAR values were then measured under the basal conditions with sequential addition of 15 mM glucose, 2 μM oligomycin, and 50 mM Rot plus 1 M AA (FIG. 20D). Specific parameters for glycolysis, glycolytic capacity and glycolytic reserve were calculated and data are presented as the mean±sd (n=18) from three independent experiments (FIG. 20E). FIG. 20F shows select metabolite levels. ImKCs were treated with DPI for 6 hrs and select metabolites in the glycolytic pathway and TCA cycle were quantified by LC-MS. Data are presented as the mean±sd (n=4). P values were calculated by student t-test. * P<0.05, ** P<0.01, *** P<0.001, **** P<0.0001.

FIGS. 21A-21I show DPI stimulates glycolysis through GPR3 and β-arrestin2. FIGS. 21A-21B show DPI-stimulated glycolysis is independent of the NOX activity. Wildtype (WT) and p47phox^{-/-} BMDMs were seeded and incubated without or with DPI (50 and 500 nM) for 24 hrs and ECAR was measured by Seahorse analyzer (FIG. 21A). WT BMDMs were seeded and incubated without or with DPI (500 nM) in the absence or the presence of NOX inhibitor apocynin (100 μM) for 24 hrs and ECAR was measured by Seahorse analyzer (FIG. 21B). Data are presented as the mean±sd (n=15) from three independent experiments. FIG. 21C shows the effect of DPI on glucose uptake in WT and p47phox^{-/-} BMDMs. BMDMs were treated with DMSO or DPI (50 and 500 nM) for 24 hrs in the presence of the fluorescent glucose analog 2-NBDG. The mean fluorescence intensity (MFI) of 2-NBDG in cells was measured by flow cytometry and normalized to DMSO controls of wildtype BMDMs. Data are presented as the mean±sd (n=3). FIG. 21D shows that DPI-stimulated glycolysis requires GPR3. ImKCs were transfected with siRNA specific for Gpr3 or a scramble siRNA as control. 48 hrs later, transfected ImKCs were seeded and incubated without or with DPI (50 and 500 nM) for 24 hrs and ECAR was measured by Seahorse analyzer. Data are presented as the mean±sd (n=15) from three independent experiments. FIG. 21E shows that transfected ImKCs were incubated without or with DPI (50 and 500 nM) for 24 hrs in the presence of 2-NBDG to measure the glucose uptake. Data are presented as the mean±sd (n=4). FIG. 21F shows that ImKCs were incubated with DMSO, DPI (500 nM) or S1P (3 mM) for 24 hrs and ECAR was measured by Seahorse analyzer. Data are presented as the mean±sd (n=12). FIG. 21G shows that DPI-stimulated glycolysis requires β-arrestin-2. Abbr2^{-/-} ImKC were constructed by CRISPR-Cas9-mediated gene editing. WT and Abbr2^{-/-} ImKCs were seeded and incubated without or with DPI (50 and 500 nM) for 24 hrs and ECAR was measured by Seahorse analyzer. Data are presented as the mean±sd (n=15) from three independent experiments. FIG. 21H shows that WT andAbbr2^{-/-} ImKCs were incubated without or with DPI (50 and 500 nM) for 24 hrs in the presence of 2-NBDG to measure the glucose uptake. Data are presented as the mean±sd (n=4). FIG. 21I shows that DPI induces β-arrestin2 translocation to cytoplasm membrane. ImKCs were transfected with Abbr2-GFP fusion gene and stimulated with DMSO, DPI (50 nM), or S1P (3 mM). The GFP signal was captured with a TIRF microscope at indicated time points. Shown are representative data of GFP signal at 0 min and 10 min, and merged signal from three independent experiments. P values were calculated by student t-test. * P<0.05, ** P<0.01, *** P<0.001, **** P<0.0001.

FIGS. 22A-22E show that DPI stimulates rapid increase in glycolytic activity through the formation of GPR3-β-arrestin2-GAPDH-PKM2 enzymatic super complex. FIG. 22A shows Co-IP of β-arrestin2 with ERK1/2, enolase, GAPDH, and PKM2. ImKCs were transfected with β-arrestin2 and then treated with or without 50 nM DPI for 6 hrs. Cell lysates were precipitated with anti-β-arrestin2 and the precipitates were analyzed by Western blotting for the indicated proteins. Shown are representative data from one of the three experiments. FIG. 22B shows that DPI-stimulated glycolysis requires PKM2. BMDMs were prepared from wild-type and Pkm^{-/-} mice, seeded and incubated with or without DPI (50 and 500 nM) for 24 hrs and ECAR was measured by Seahorse analyzer. Data are presented as the mean±sd (n=15) from three independent experiments. FIG. 22C shows that WT and Pkm^{-/-} BMDMs were seeded and incubated with or without DPI (50 and 500 nM) for 24 hrs in the presence of 2-NBDG to measure the glucose uptake. Data are presented as the mean±sd (n=4). FIGS. 22D-22E show that DPI stimulates enzymatic activities of PKM2 and GAPDH. Wildtype and Abbr2^{-/-} ImKCs were treated with DPI (500 nM) for 6 hrs and the enzymatic activities of PKM2 (FIG. 22D) and GAPDH (FIG. 22E) were measured by colorimetric assay kits. Data are presented as the mean±sd (n=6). P values were calculated by student t-test. * P<0.05, ** P<0.01, *** P<0.001, **** P<0.0001.

FIGS. 23A-23D show that DPI stimulates sustained increase in glycolytic activity through nuclear translocation of PKM2 and transcriptional activation. FIG. 23A shows that DPI-induced transcription of glycolytic genes requires PKM2. WT and Pkm^{-/-} BMDMs were not treated or treated with DPI (50 and 500 nM) for 24 hrs. The transcript levels of Pkm, Ldha, Hk2 and c-Myc were measured by real-time qPCR. Data were collected from two independent experiments with 3 biological replicates per group. Transcriptional level was normalized to β-actin first and then to DMSO control. Data are presented as the mean±sd. FIG. 23B shows induction of dimeric PKM2 by DPI. ImKCs were not treated or treated with DPI (50 and 500 nM) for 6 or 12 hrs. Cell lysates were run on native PAGE gel and analyzed by Western blotting. Shown are representative data from two independent experiments. FIG. 23C shows that DPI induces nuclear translocation of PKM2. ImKCs and human primary KCs were not treated or treated with DPI (50 nM) for 24 hrs, stained with anti-PKM2 and DAPI, followed by confocal imaging. Shown are representative images from two independent experiments. Enlarged areas are boxed. FIG. 23D shows that DPI stimulates transactivation of c-Myc. c-Myc luciferase reporter plasmid was transfected into WT and Pkm^{-/-} BMDMs. Transfected cells were not treated or treated with DPI (50 and 500 nM) for 6 hrs and luciferase activities were measured. Data are presented as the mean±sd (n=5). P values were calculated by student t-test. * P<0.05, ** P<0.01, *** P<0.001, **** P<0.0001.

FIGS. 24A-24H show that DPI inhibits HFD-induced obesity and liver pathogenesis through PKM2 expression in Kupffer cells. FIGS. 24A-24B shows that DPI prevents weight gain in mice fed with HFD. Male B6 mice at 5 weeks of age were fed with HFD or normal chow diet (ND) for a total of 8 weeks. Three weeks after HFD (arrow), half of mice were given DPI in vehicle (2 mg/kg) and the other half were given vehicle alone every five days for a total of 6 doses. The body weight (FIG. 24A) and food consumption (FIG. 24B) were monitored weekly. Data are presented as the mean±sd from three independent experiments with 12-15 mice per group. FIG. 24C shows the weights of eWAT and iWAT of mice after 8 weeks on HFD. Each dot represents one mouse. FIG. 24D show fast glucose assay. Mice from FIG. 24A at week 7 plus 3 days were starved overnight (12˜16 hrs) with only water. Glucose (1 mg/kg) was injected intraperitoneally and blood glucose levels were monitored at the indicated time. AUC (right panel) were calculated for statistics. FIG. 24E shows serum levels of AST and ALT. Sera from mice in FIG. 24A were collected and activities of AST and ALT were measured by colorimetric assay kits (Sigma). FIG. 24F shows comparison of H&E staining of liver sections from HFD mice treated with vehicle or DPI after 8 weeks on HFD. Shown are representative H&E staining from one mouse per group from FIG. 24A. Arrows point to lipid droplets. Scale bar: 100 μm. FIGS. 24G-24H show DPI's effect on KC-specific Pkm^{-/-} mice fed with HFD. Male KC-specific Pkm^{-/-} mice at the age of 5 weeks were fed with HFD for a total of 8 weeks. Three weeks after HFD, half of the mice were given DPI in vehicle (2 mg/kg) and the other half were given vehicle every 5 days for a total of 6 doses. Body weights were monitored weekly (FIG. 24G). Data are presented as the mean±sd from two independent experiments with 6 mice per group. Comparison of H&E staining of liver sections after 8 weeks on HFD. Shown are representative H&E staining from one mouse per group. Arrows in FIG. 24F and FIG. 24H point to lipid droplets. Scale bar: 100 μm. P values were calculated by student t-test. * P<0.05, ** P<0.01, *** P<0.001, **** P<0.0001.

FIGS. 25A-25D show that DPI upregulates glycolysis and suppresses inflammatory responses of Kupffer cells in HFD-fed mice. FIG. 25A shows comparison of gene expression in KCs isolated from mice fed with ND or HFD. Single cell suspension was prepared from mice from FIG. 24A after 8 weeks on HFD (6 mice per group), stained with anti-F4/80, anti-CD11b and anti-Gr-1. F4/80⁺CD11b⁺Gr1^{low} macrophages were purified by cell sorting followed by RNAseq. Shown are differentially expressed genes among the three groups. FIG. 25B shows functional enrichment analysis of DEGs based on comparison of KCs from HFD-fed and ND-fed mice or from HFD-fed mice treated with DPI or vehicle. FIG. 25C shows GSEA of gene expression profiles of KCs either from HFD and ND mice, or from HFD mice treated with DPI or vehicle. Graphs in FIG. 25B and FIG. 25C indicate up- and down-regulated pathways as labeled. FIG. 25D shows macrophage polarization index analysis based on the expression profile in FIG. 25A with the online software MacSpectrum (see the World Wide Web at macspectrum.uconn.edu). M1-type polarization is expressed as positive scores whereas M2-type polarization is expressed as negative scores.

FIGS. 26A-26H shows that DPI upregulates glycolysis and suppresses inflammatory responses of Kupffer cells from patients with NAFLD. FIGS. 26A-26D show scRNAseq analysis of the macrophage populations. A total of 5,497 macrophages based on the expressing of CD14 and CD68 (cluster 5, 8 and 12 in FIG. 35A) were subjected to clustering analysis by tSNE. A total of 7 clusters were identified (FIG. 26A). Relative proportion of each cluster in each sample was calculated and shown (FIG. 26B). Each cluster was annotated based on the expression of typical markers as shown by dot plot (FIG. 26C) and heatmap (FIG. 26D). FIG. 26E shows trajectory inference of the liver macrophages by slingshot (Street et al. 2018). FIG. 26F shows GO enrichment analysis of DEGs between C3 and C1 and C2. FIGS. 26G-26H show comparison of gene expression changes induced by DPI in primary KCs isolated from NAFLD liver biopsies. CD14⁺ KCs were sorted from single cell suspension of NAFLD human liver biopsies (n=2) and treated with DMSO or DPI (500 nM) for 24 hrs, followed by RNAseq to quantify gene expression. Shown are the expression changes of glycolytic genes and DAM markers (FIG. 26G) and GO enrichment analysis of DEGs induced by DPI in KCs (FIG. 26H). Graphs in FIG. 26F and FIG. 26H indicate up- and down-regulated pathways as labeled.

FIGS. 27A-27C show that DPI stimulates both rapid and sustained increase in glycolysis in macrophages. FIG. 27A shows that DPI stimulates transcription of glycolytic genes in human primary macrophages following treatment with 50 nM DPI for 24 hours. Heatmap of transcript levels is based on reanalysis of RNAseq data from Hu et al. 2021. FIG. 27B shows that DPI stimulates expression of glycolytic enzymes at protein level as measured by Western blotting. Total protein lysates were isolated from either mouse ImKCs with or without DPI treatment for 6 and 12 hrs or human primary macrophages with or without DPI treatment for 12 hrs at the indicated concentrations. Equal amounts of total proteins from whole-cell lysates were subjected to Western blotting analysis. β-actin was used as a loading control. Shown are representative data of two independent experiments. FIG. 27C shows metabolite analysis in ImKCs. ImKCs were treated with DPI (500 nM) for 24 hrs and the select metabolites were quantified by LC-MS. Shown are representative data of two independent experiments. P values were calculated by student t-test. * P<0.05, ** P<0.01. n.s. not significant.

FIGS. 28A-28I show that DPI stimulates glycolysis through GPR3 and β-arrestin2. FIGS. 28A-28B show that DPI-stimulated glycolysis is independent of the NOX activity. Wild-type (WT) and p47phox^{-/-} BMDMs were seeded and incubated without or with DPI (50 and 500 nM) for 24 hrs and ECAR was measured by Seahorse analyzer. Specific parameters for glycolytic capacity and glycolytic reserve were calculated and summarized based on two independent experiments. Data are presented as the mean±sd (n=15) from three independent experiments. FIG. 28C shows Western blotting of GPR3 in ImKCs transfected with scramble or siGpr3. FIGS. 28D-28E show that ImKCs were transfected with siRNA specific for Gpr3 or scramble siRNA. 48 hours later, transfected ImKCs were seeded and incubated without or with DPI (50 and 500 nM) for 24 hrs and ECAR was measured by Seahorse analyzer. Data are presented as the mean±sd (n=15) from three independent experiments. FIG. 28F shows Western blotting of β-arrestin2 in wild-type or Abbr2^{-/-} ImKCs. FIGS. 28G-28H show that WT and Abbr2^{-/-} ImKCs were seeded and incubated without or with DPI (50 and 500 nM) for 24 hrs and ECAR was measured by Seahorse analyzer. Data are presented as the mean±sd (n=15) from three independent experiments. FIG. 28I show that BMDMs were transfected with Arrb2-GFP fusion gene and stimulated with DMSO or DPI (50 nM). The GFP signal was captured with a TIRF microscope at indicated time points. Shown are representative data of GFP signal at 0 min and 10 min, and the merged signal from three independent experiments. P values were calculated by student t-test. * P<0.05, ** P<0.01, *** P<0.001, **** P<0.0001. n.s. not significant.

FIG. 29A-29D show that DPI stimulates rapid increase in glycolytic activity through the formation of GPR3-β-arrestin-GAPDH-PKM2 enzymatic super complex. FIGS. 29A-29B show comparison of DPI's effect on glycolysis in wild-type and Pkm^{-/-} BMDMs. BMDMs were generated from wildtype and PKM2^{-/-} mice, seeded and incubated with or without DPI (50 and 500 nM) for 24 hrs and ECAR was measured by Seahorse analyzer. Data are presented as the mean±sd (n=15) from three independent experiments. FIGS. 29C-29D show activation of PKM2 and GAPDH enzymatic activity by DPI is inhibited by ERK1/2 inhibitor. ImKCs were treated with DMSO or DPI alone (500 nM) or DPI plus ERK1/2 inhibitor (SCH772984, 1 mM) for 6 hrs and the enzymatic activities of PKM2 (c) and GAPDH (d) were measured by colorimetric assay kits (Biovision). Data are presented as the mean±sd (n=6). P values were calculated by student t-test. * P<0.05, ** P<0.01, *** P<0.001, **** P<0.0001. n.s. not significant.

FIGS. 30A-30B show that DPI stimulates sustained increase in glycolytic activity through formation of dimeric PKM2. FIG. 30A shows induction of dimeric PKM2 by DPI. ImKCs were not treated or treated with DPI (50 and 500 nM) for 6 or 12 hrs. Cells were treated with crosslinking agent DSS and lysed. Lysates were run on SDS-PAGE and analyzed by Western blotting. Shown are representative data from two independent experiments. FIG. 30B shows that phosphorylation of ERK1/2 is inhibited by SCH772984 in the presence of DPI. ImKCs were not treated or treated with DPI (50 and 500 nM) in the presence or the absence of SCH772984 for 12 hrs. Cells were lysed and analyzed for total ERK1/2 and phosphorylated ERK1/2 by Western blotting. Shown are representative data from two independent experiments. P values were calculated by student t-test. * P<0.05, ** P<0.01, *** P<0.001, **** P<0.0001. n.s. not significant.

FIG. 31 shows that DPI stimulates metabolism of blood glucose in mice. C57BL/6 mice at 10 weeks of age were given a single injection of DPI (2 mg/kg) intraperitoneally. Six hrs later (−360 min), mice were injected intraperitoneally with glucose (1.5 mg/kg). Blood glucose levels were monitored at the indicated time. Data are presented as the mean±sd with 5 mice per group. P values were calculated by student t-test. * P<0.05, ** P<0.01, *** P<0.001.

FIGS. 32A-32E show that DPI inhibits HFD-induced obesity and liver pathogenesis. FIGS. 32A-32B show that male B6 mice at 5 weeks of age were fed with HFD for a total of 16 weeks. Nine weeks after HFD (arrow), half of the mice were dosed with vehicle and the other half with DPI in vehicle (2 mg/kg) every 5 days with a total of 6 doses. The weight (FIG. 32A) and food consumption (FIG. 32B) were monitored weekly. Data are presented as the mean±sd from two independent experiments with 9-10 mice per group. FIG. 32C shows the weights of eWAT and iWAT after 16 weeks on HFD. FIG. 32D shows fast glucose assay. At week 15 plus 3 days, mice from FIG. 32A were starved overnight (12˜16 hrs) with only water. Glucose (1 mg/kg) was injected intraperitoneally and blood glucose levels were measured at the indicated time. AUC were calculated for statistics (right panel). FIG. 32E shows comparison of H&E and trichrome staining of liver sections from HFD mice treated with vehicle or DPI. Shown are representative H&E staining from one mouse per group from a. Scale bar: 100 m. P values were calculated by student t-test. * P<0.05, ** P<0.01, *** P<0.001. n.s. not significant.

FIG. 33 shows expression of PKM2 and PKM1 in human and mouse Kupffer cells and hepatocytes. scRNAseq data from normal human liver and mouse liver were reanalyzed for expression of PKM2 and PKM1 as well as markers of macrophages (VSIG4 or F4/80) and hepatocytes (APOC3 or Apoc3) using featureplot in Seurat package. In the mouse dataset, the small population of hepatocytes (Apoc3+) is due to the removal of hepatocytes in the process of enriching immune cells for scRNAseq.

FIGS. 34A-34C show effect of DPI on Kupffer cell-specific Pkm^{-/-} mice on HFD. FIG. 34A shows fast glucose assay. KC-specific Pkm^{-/-} mice were given HFD for a total of 8 weeks. Three weeks after HFD, half of the mice were given DPI in vehicle (2 mg/kg) and the other half were given vehicle every 5 days for a total of 6 doses. At 7 weeks plus 3 days, mice were starved overnight (12˜16 hrs) with only water. Glucose (1 mg/kg) was injected intraperitoneally and blood glucose levels were monitored at the indicated time. FIG. 34B shows the weights of eWAT and iWAT after 8 weeks on HFD. FIG. 34C shows serum levels of AST and ALT. Sera from mice were collected at the end of HFD feeding and activities of AST and ALT were measured by colorimetric assay kits (Sigma). Shown are representative data from two independent experiments with 5˜6 mice per group. P values were calculated by student t-test. n.s. not significant. * P<0.05.

FIGS. 35A-35D show Single cell RNAseq analysis of immune cells from biopsies of healthy and NAFLD human livers. A total 47,724 immune cells from 3 healthy and 3 NAFLD human liver biopsies were clustered into 14 clusters by tSNE (FIG. 35A). Each cluster was annotated based on the expression of typical markers as T and B cells, NK cells, macrophages, neutrophils and dendritic cells as shown by dot plotting (FIG. 35B). Cell fraction of each cluster (FIG. 35C) and relative proportion of each cluster in each sample (FIG. 35D) were calculated and shown.

FIG. 36 shows GO enrichment analysis of DEGs of different liver macrophage subpopulations. DEGs were identified using the function of FindMarkers in Seurat package between different clusters as indicated with setting min.fct to 0.25 and logfc.threshold to 0.25. Up- and down-regulated DEG were applied to GO ontology enrichment analysis by the online tool DAVID (see the World Wide Web at david.ncifcrf.gov). Shown are the selected top GO terms and p values based on the significance and redundance. Graphs indicate up- and down-regulated pathways as labeled.

### DETAILED DESCRIPTION OF THE INVENTION

Macrophages are remarkably plastic and in response to different local stimuli can polarize toward multi-dimensional spectrum of phenotypes, including the pro-inflammatory M1-like and the anti-inflammatory M2-like states. Using a high throughput phenotypic screen, ˜300 compounds that potently activated primary human macrophages toward either pro-inflammatory (M1-like) or anti-inflammatory (M2-like) state were identified from a library of ˜4000 FDA-approved drugs, bioactive compounds and natural products. Among the hits, ˜30 were capable of reprogramming M1-like macrophages toward M2-like state and another ˜20 were capable of reprogramming M2-like macrophages toward M1-like state. Transcriptional analysis of 34 non-redundant hits on macrophage reprogramming by RNA-seq identified shared pathways through which the selected hits modulate macrophage activation, as well as new unique targets and pathways by which individual compound stimulates macrophage activation. One M1-activating compound, thiostrepton, was further shown to reprogram tumor-associated macrophages toward M1-like state in mice and exhibit potent anti-tumor activity either alone or in combination with an antibody therapeutic. Described herein are new compounds, targets and pathways involved in macrophage activation. The methods described herein provide a valuable resource not only for studying the macrophage biology but also for developing novel therapeutics or repositioning known drugs for treating diseases through modulating macrophage activation.

Macrophages are a key class of phagocytic cells that readily engulf and degrade dying/dead cells and invading bacteria and viruses. As such, macrophages play an essential role in development, tissue homeostasis and repair, and immunity. Consistently, macrophages are generated during early ontogeny and throughout the adult life. In mammals, the first wave of macrophages is generated from the yolk sac and gives rise to macrophages in the central nervous system, i.e., microglia, for example. The second wave of macrophages is generated from fetal liver and give rise to alveolar macrophages in the lung and Kupffer cells in the liver among others. After birth, macrophages are generated from the bone marrow where hematopoietic stem cells give rise to monocytes, which differentiate into tissue resident macrophages upon migration from blood into specific tissues.

A remarkable feature of macrophages is their plasticity: the ability to respond to local stimuli to acquire different phenotypes and functions so as to respond to changing physiological needs. Macrophages from different tissues exhibit different phenotypes and functions. For example, Kupffer cells in the liver function in the degradation of toxic and waste products as well as in the maintenance of metabolic homeostasis, whereas alveolar macrophages in the lung function in removal of dust, microorganisms, and surfactants from the respiratory surfaces despite their common origin from fetal liver. Within the same tissue, macrophages are heterogeneous and can change phenotypes and functions in response to changing local tissue environment. For example, macrophages can eliminate antibody-bound tumor cells through Fc receptor-mediated phagocytosis (antibody-dependent cellular phagocytosis or ADCP). However, once adapted to the tumor microenvironment, the tumor-associated macrophages (TAM) suppress anti-tumor immune responses and promote tumor growth and metastasis.

Macrophage plasticity underlies their ability to be activated toward a spectrum of phenotypes and acquire diverse functions. One extreme is the classically activated pro-inflammatory M1 macrophages and the other extreme is the alternatively activated anti-inflammatory M2 macrophages. By expressing inflammatory cytokines, such as IFNγ and TNFα, and reactive oxygen species, M1 macrophages mediate anti-microbial and anti-tumor responses, but can also cause inflammation and tissue damage if hyper-activated. In contrast, by expressing anti-inflammatory cytokines, such as IL-10, TGFβ and arginase, M2 macrophages mediate tissue repair, but can also mediate fibrosis if dysregulated. While M1 and M2 serves to define the opposite activating states of macrophages in simplistic manner, most macrophages exhibit multi-dimensional spectrum of phenotypes in response to various physiological and pathological signals. By transcriptional profiling of human monocyte-derived macrophages (hMDMs) in response to 29 different stimuli, such as pro- and anti-inflammatory cytokines, 49 gene expression modules that are associated with macrophage activation were identified. Many aspects of macrophage activation/plasticity remain poorly defined. In particular, how small molecules modulate macrophage activation remains to be elucidated.

Because of their critical function in maintaining tissue homeostasis and repair, dysregulation of macrophage polarization has been implicated in contributing to many human diseases including cancer, fibrosis, obesity, diabetes, and infectious, cardiovascular, inflammatory and neurodegenerative diseases. For example, TAMs are one of the most abundant immune cells present in solid tumors. Clinical and experimental studies have shown that TAMs produce various membranous and soluble factors that enhance tumor cell growth and invasion as well as suppress anti-tumor immune responses to allow cancer cells to escape immune surveillance. TAMs are derived from circulating monocytes in the tumor microenvironment, which progressively skews macrophages into the immunosuppressive state, phenotypically resembling M2-activated macrophages. Reprogramming M2-like TAMs toward M1-like macrophages is associated with expression of a strong anti-tumor activity. In a remarkable synergy, cyclophosphamide-activated macrophages efficiently eliminate leukemia cells in refractory bone marrow microenvironment in combination with monoclonal antibody therapeutics. Repolarizing TAMs toward a pro-inflammatory, anti-tumorigenic M1-like state proves an efficient approach to cancer immunotherapy either alone or in combination with antibody therapeutics. More broadly, as dysregulation of macrophage activation has emerged as a key determinant in many disease development and progression, modulation of macrophage activation could be a fruitful approach for disease intervention.

Described herein is a high throughput phenotypic screen for small molecules that activate primary human macrophages. By screening a library of 4126 compounds which include FDA-approved drugs, bioactive compounds and natural products, ˜300 potently activated M-CSF cultured macrophages toward pro-inflammatory M1-like or anti-inflammatory M2-like state (or spectrum) were identified. Among the hits, ˜30 were capable of reprogramming M2-like macrophages induced by IL4/IL13 toward pro-inflammatory M1-like macrophages and another ˜20 were capable of reprogramming M1-like macrophages induced by IFNγ/TNFα toward anti-inflammatory M2-like macrophages. By analyzing the effects of the 34 selected hits on macrophage reprogramming through RNA-seq, we identified new cellular pathways that mediate macrophage activation (or reprogramming). M1-activating compounds thiostrepton and cucurbitacin I were further shown to reprogram TAMs toward M1-like macrophages in mice and exhibit potent anti-tumor activity either alone or in combination with monoclonal antibody therapeutics. The examples herein reveal a remarkable plasticity of macrophage polarization and provides a valuable resource not only for studying the macrophage biology but also for developing novel therapeutics or repositioning known drugs for treating diseases through macrophage reprogramming. Furthermore, the phenotypic screen can be extended to much larger compound libraries and in combination with transcriptional profiling is a powerful approach to elucidate the mechanism of action of small molecule compounds in macrophage polarization for precision disease intervention.

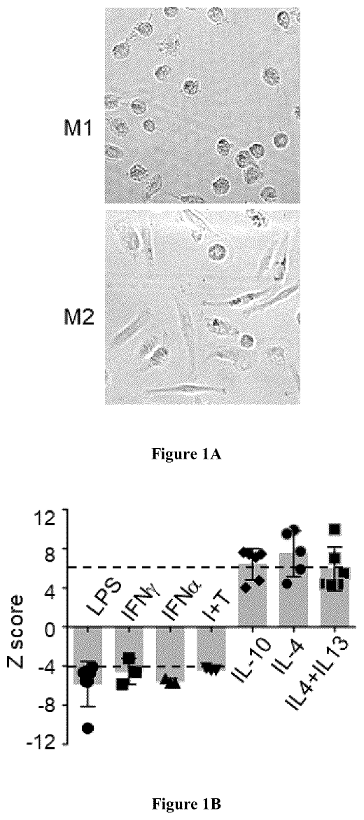

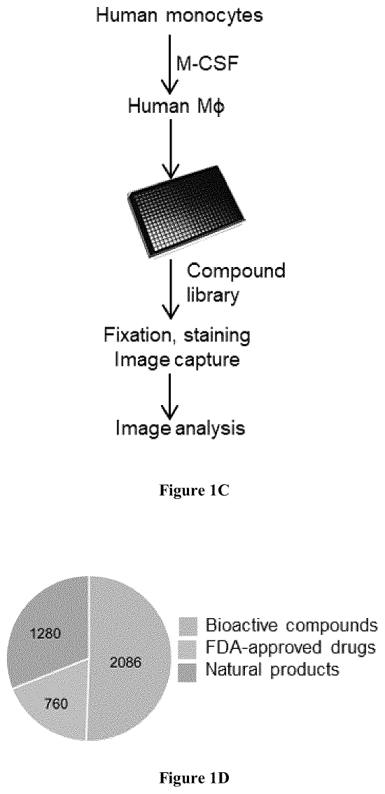

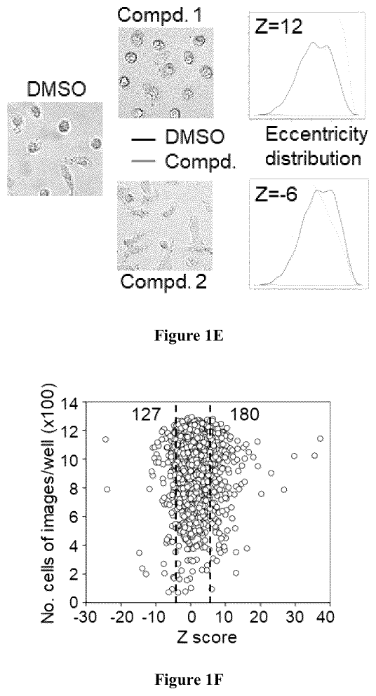

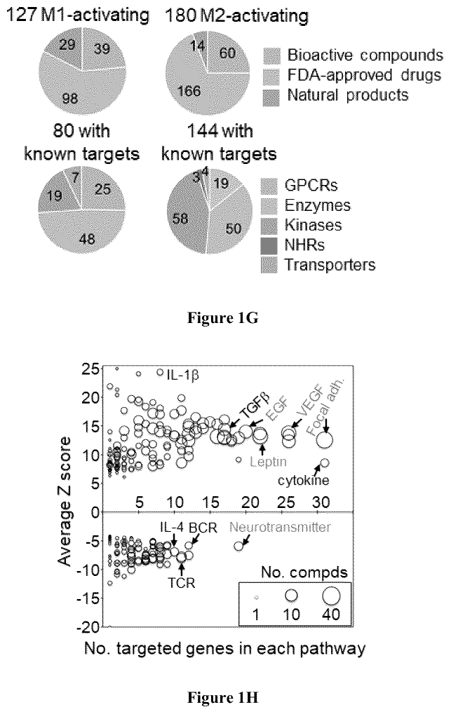

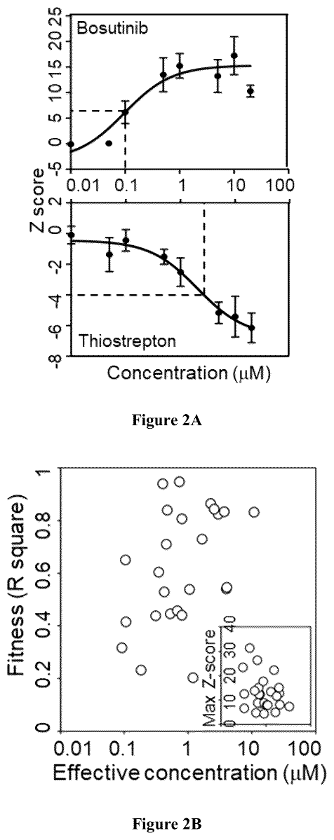

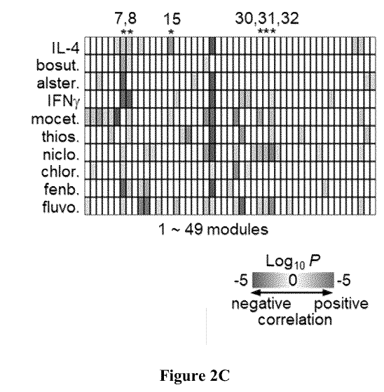

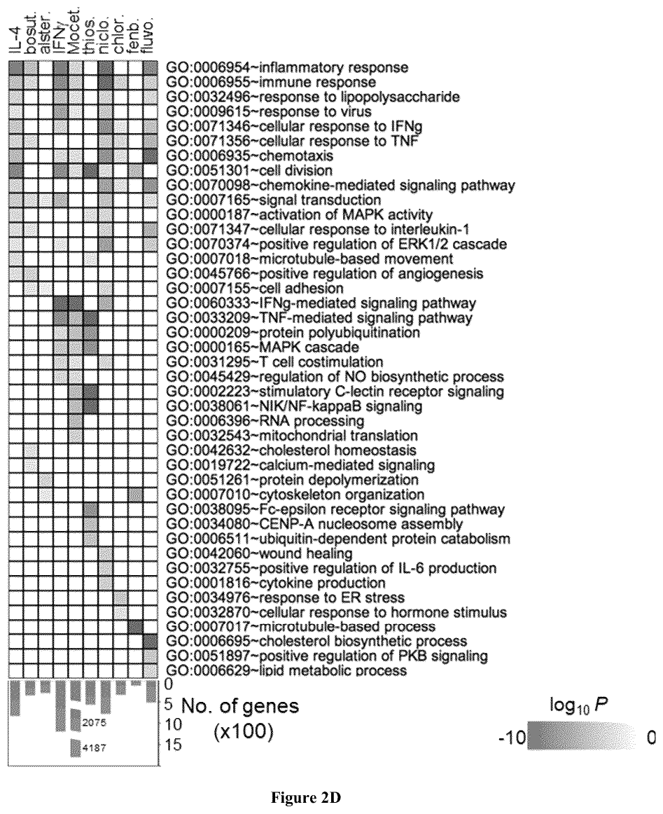

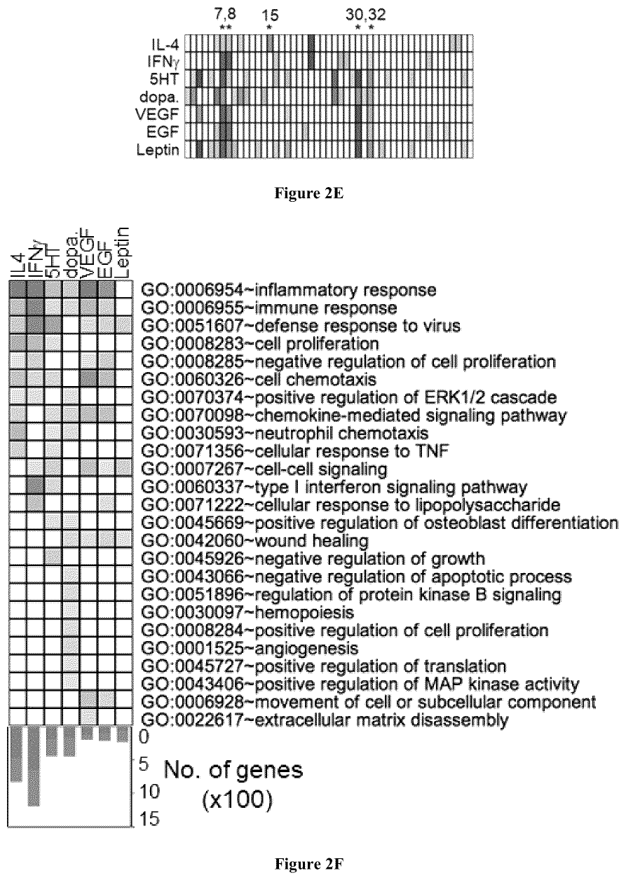

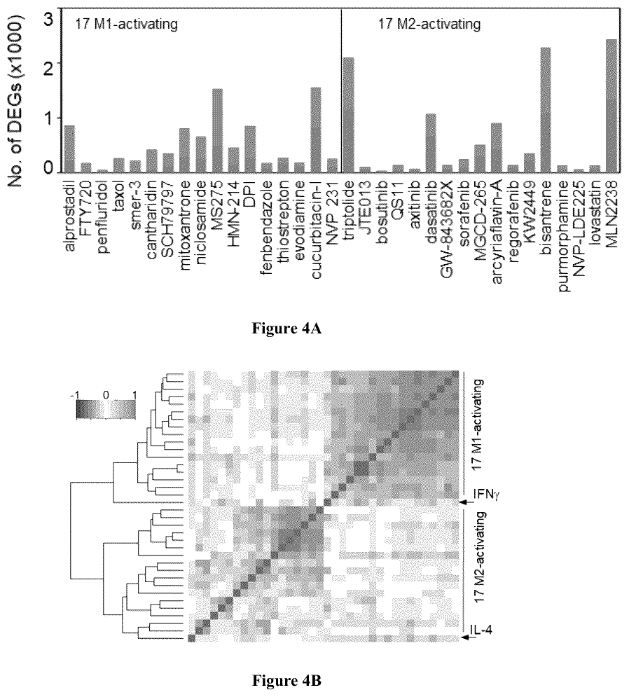

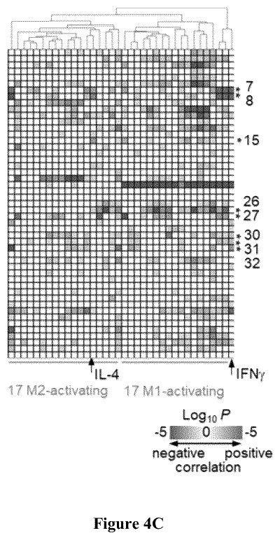

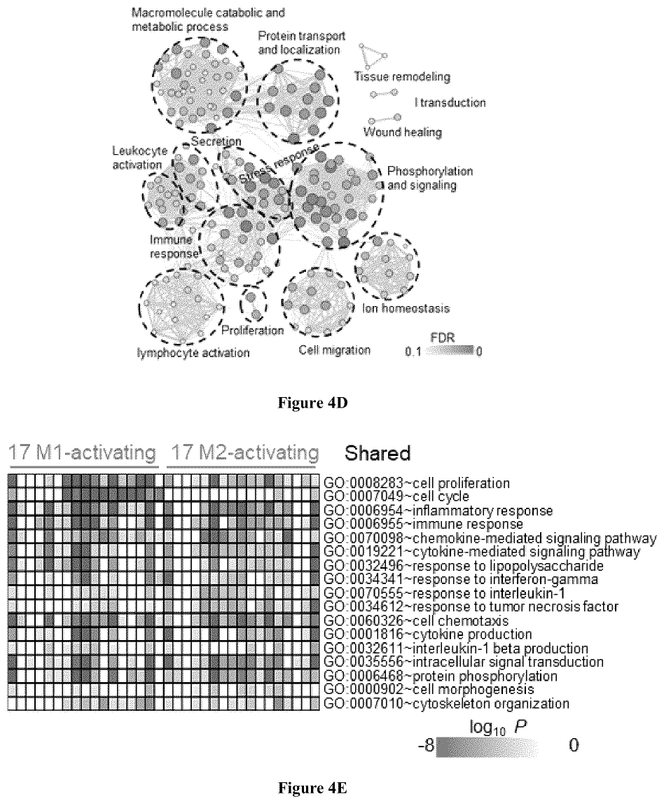

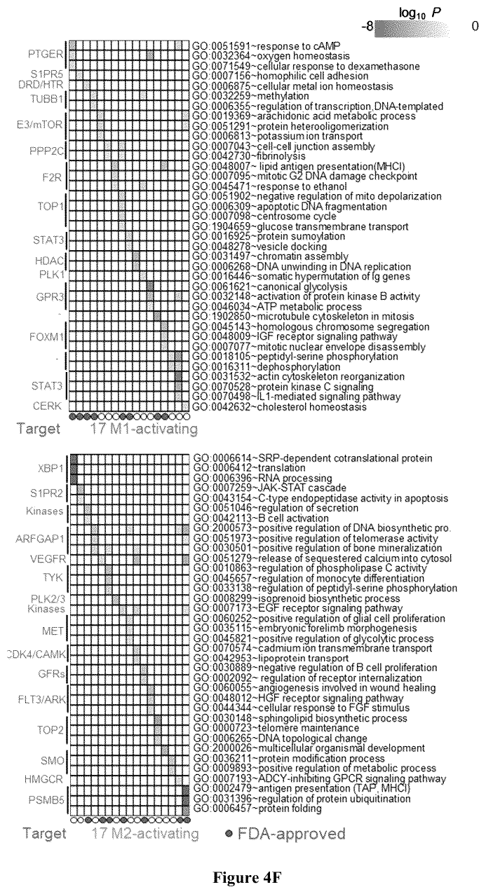

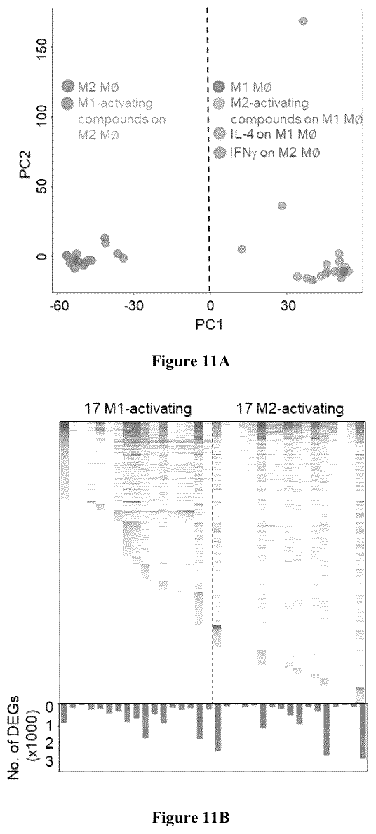

The high throughput phenotypic screen described herein is based on macrophage cell shape changes in response to compounds. Cell shape change is a valid phenotypic profiling of macrophage activation based on the following considerations. First, cell shape changes are mediated by changes in cytoskeleton dynamics and are known to associate with different states of cell function in general. More specifically, both mouse and human macrophages exhibit dramatically different cell shapes following activation into different phenotypes in vitro: an elongated shape for M2-like macrophages and round shape for M1-like macrophages. Consistently, we showed that known M1-activating stimuli LPS, IFNγ and TNFα induced round shape of differentiated macrophages whereas known M2-activating stimuli IL4, IL13 and IL10 induced elongated cell shape of differentiated macrophages (FIG. 1). Similarly, GM-CSF-induced round human macrophages and M-CSF-induced elongated human macrophages exhibited M1-like and M2-like phenotypes, respectively, based on cytokine profiles, and the genome-wide gene expression. Second, it has been shown that inducing cytoskeleton changes by extracellular stress or drug paclitaxel lead to macrophage polarization. In the examples herein, we also identified several compounds/drugs that modulate macrophage morphology by directly regulating actin-cytoskeleton, including paclitaxel as well as other M1-activating compounds: cytochalasin-B, fenbendazole, parbendazole, methiazole, and M2-activating compounds: podofilox, colchicine and vinblastine sulfate. Analysis of human macrophage responses to fenbendazole and paclitaxel further confirmed that both drugs activated macrophages toward M1-like phenotype at both the transcriptional and translational level (FIGS. 2C, 4, 9C, and 11). Third, although we used cell shape change as a high throughput readout in the initial phenotypic screen, we confirmed the effects of over 40 selected compounds on macrophage activation at the whole genomic level by RNA-seq (FIGS. 2 and 4) and protein expression of typical M1 and M2 markers by flow cytometry (FIG. 11). As expected, pathway analysis of DEGs identified cell morphogenesis and cytoskeleton organization as major GO terms that are regulated by the compounds (FIG. 4C). Thus, a cell shape-based phenotypical profiling is a valid approach to screen for small molecule compounds that activate human macrophages. The data in the Examples herein is a first proof-of-concept large scale screen using primary human macrophages. The screen can be extended to much larger compound libraries as the microscopy-based cell shape profiling can be easily scaled up. As further discussed below, the combination of the phenotypic screen and transcriptional analysis could be a powerful approach to identify compounds and their mechanisms of action in macrophage activation for new drug development.

The data herein identifies compounds, targets and pathways that mediate macrophage activation and sheds new light on the underlying molecular mechanisms. In our library, many compounds have known protein targets. Based on functional pathway enrichment analysis of protein targets of M1- or M2-activating compounds, we identified known pathways, such as cytokine, in macrophage activation. More importantly, we identified new pathways, including leptin, VEGF, EGF and neurotransmitter pathways, which mediate macrophage activation. Although studies have shown these pathways in macrophage function, their effects on macrophage activation and underlying mechanisms are unknown. Our transcriptional analysis of macrophages suggests that the ligands of these pathways activate macrophage by regulating gene expression of both typical M1 and M2 modules. For example, in hMDMs, leptin upregulates the expression of typical M1 modules induced by IFNγ while suppresses the expression of chronic inflammation TPP modules (FIG. 2). Notably, the ligands of serotonin transporter and receptors, histamine transporter and receptors, dopamine transporter and receptors, and adrenoceptors all stimulated M1-like macrophage activation, shedding light on the cross-talk between neuronal and immune systems and the potential roles of macrophage activation in neurological diseases.

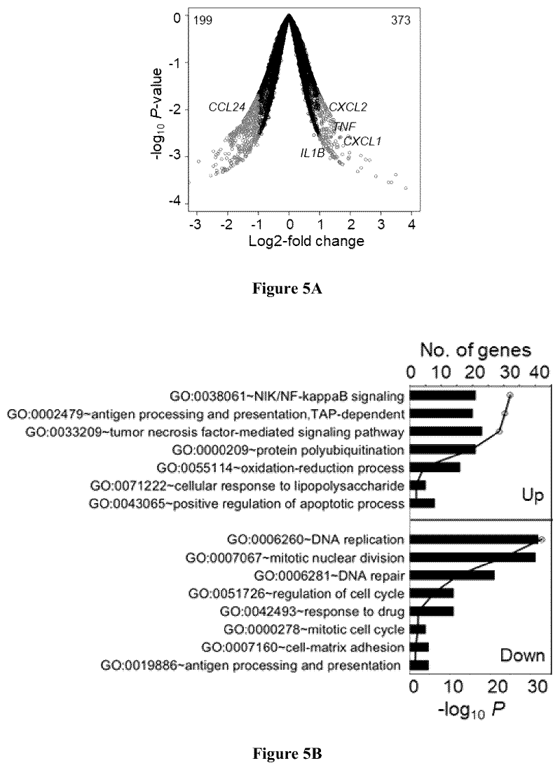

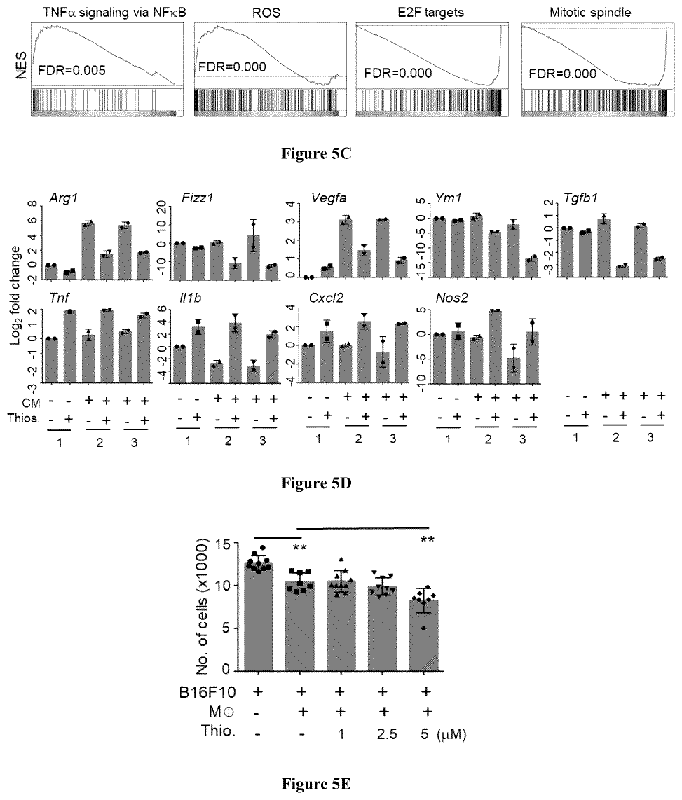

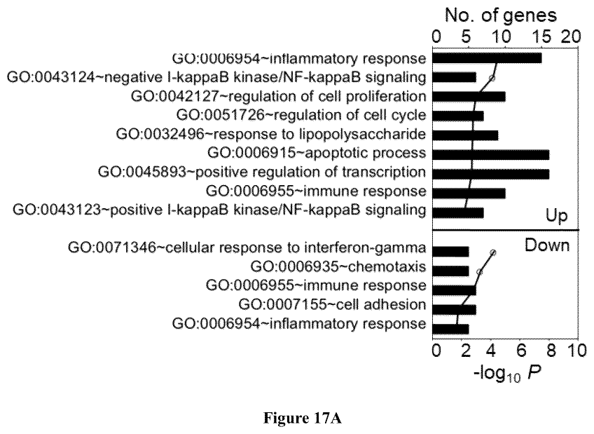

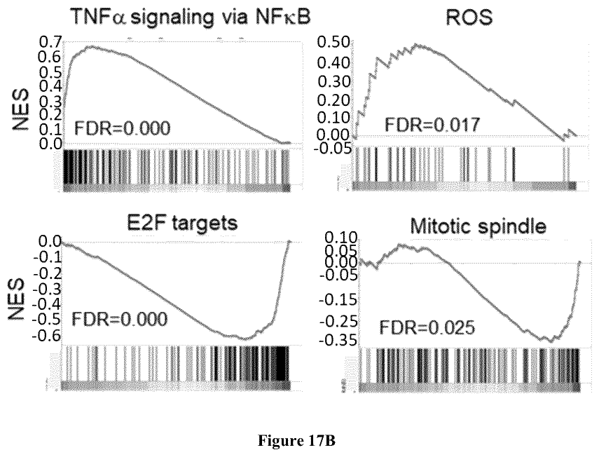

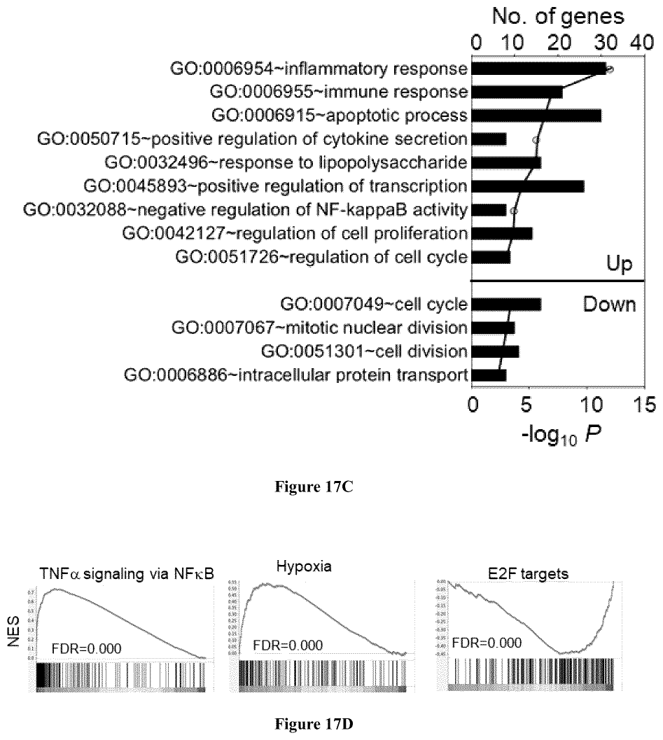

Macrophages exhibit a multi-dimensional spectrum of phenotypes beyond M1 and M2. Our identification of a diverse panel of macrophage-activating compounds that target GPCRs, enzymes, kinases, nuclear hormone receptors (NHRs), and transporters (FIG. 1G) adds to the molecular basis of macrophage plasticity and further identifies new pathways in macrophage activation. Our extensive transcriptional analysis with over 40 selected compounds identifies how each compound stimulates macrophage activation through shared mechanisms and unique pathways. All compounds modulated macrophage activation through common pathways such as inflammatory response, immune response, chemokine- and cytokine-mediated signaling pathways. Furthermore, each compound induced unique transcriptional responses of macrophages through their specific cellular targets; and many of these unique pathways are not known to mediate macrophage activation. For example, thiostrepton has been shown to have antiproliferative activity in cancer cells by inhibiting proteasome function or FOXM. In both human and mouse macrophages, thiostrepton upregulated expression of pro-inflammatory genes, as well as genes associated with IFN/NFκB pathway and oxidative-reduction process (FIGS. 5 and 17). The transcriptional analysis also revealed that most of the identified compounds stimulate macrophage activation through modulating a fraction of M1- or M2-specific gene modules as well as common denominators that are induced by M1- or M2-activating cytokines (FIGS. 4C, and 4D). The milder effect is expected as individual compound only regulates specific signaling pathways through relevant protein targets (FIG. 4E). These observations further shed light on the nature of macrophage activation. The identified large panel of small molecule compounds and their corresponding targets and pathways are a rich resource for further studying basic macrophage biology.

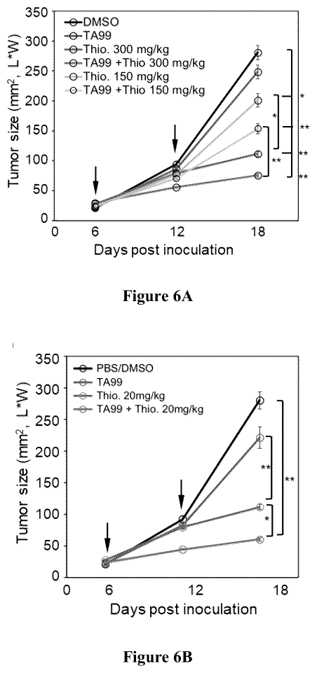

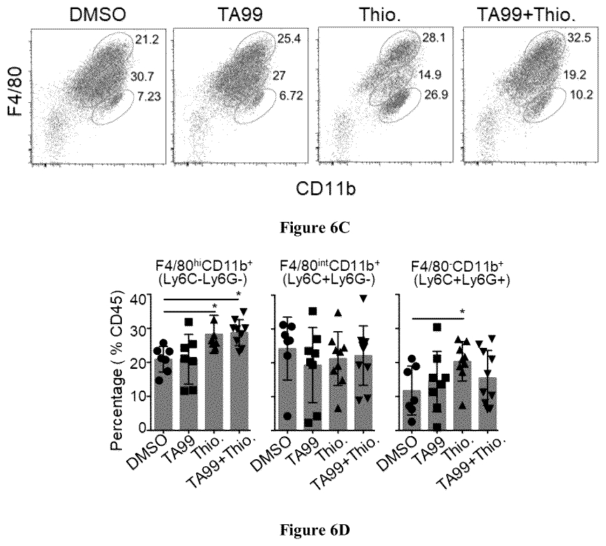

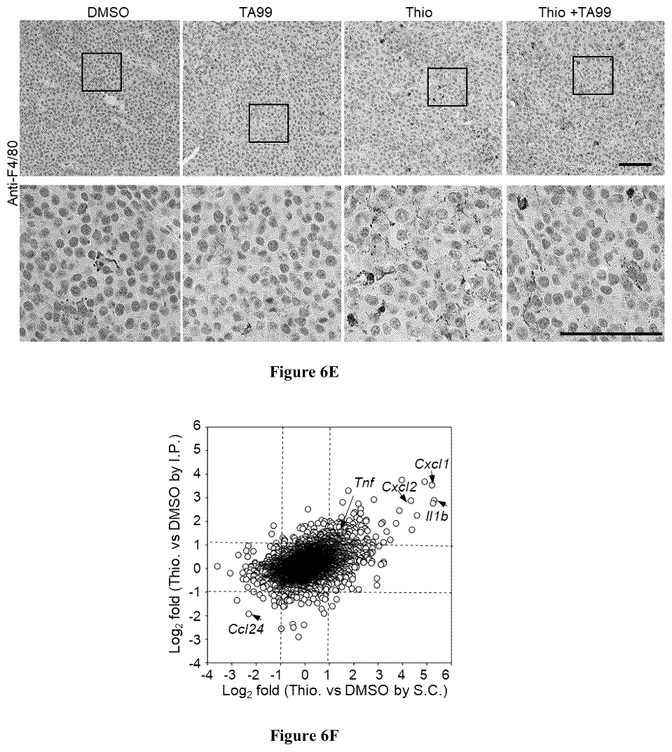

The data described herein also provides a rich resource for exploring compounds/targets/pathways for modulating macrophage activation in disease intervention. Reprogramming macrophage has emerged as a significant approach for treating a variety of diseases. Suppression or reprogramming of M2-like TAMs into M1-like macrophages by small molecule compounds is associated with induction of a strong anti-tumor activity alone or in combination with other therapeutics. Similarly, suppression or reprogramming of M1-like macrophages into M2-like state significantly inhibits the progression of inflammatory and autoimmune diseases. In this study, we confirmed M1-activating compounds thiostrepton and cucurbitacin I potently reprogrammed TAMs toward M1-like macrophages and enhanced anti-tumor activity either alone or in combination with an antibody therapeutic (FIGS. 6, 18, and 19), showing that M1-activating compounds can be explored for reprogramming M2-like macrophages for the treatment of cancer and fibrosis where M2-like macrophages play a significant role in disease processes. Similarly, M2-polarizing compounds can be explored for the treatment of inflammatory diseases by suppressing the inflammatory activities of M1-like macrophages. In complex diseases, pathogenic macrophages are known to be heterogeneous including both M1- and M2-like phenotypes, or have a transitional or intermediate phenotype with mixed characteristics of M1-like and M2-like phenotypes, or exhibit a dynamic phenotype during the disease progression. To target the desired macrophage population, it is critical to suppress the expression of signature genes/pathways in the pathogenic macrophages at the correct time window. Our identification of unique pathways modulated by each compound by transcriptional analysis provides a basis for selecting the appropriate compounds to reprogram macrophages for precision disease intervention.

### Activation of GPR3-β-Arrestin2-PKM2 Pathway in Kupffer Cells Protects Against Obesity and Liver Pathogenesis Through Enhanced Glycolysis

Increasing evidence suggests a critical role of macrophages in regulating body weight and obesity associated pathologies. However, the underlying molecular and cellular mechanisms remain to be elucidated. Here, we show that diphenyleneiodonium (DPI), an agonist of G-protein coupled receptor 3 (GPR3), stimulates both rapid and sustained increase in glycolysis at cellular level and protects mice from high fat diet (HFD) induced obesity and liver pathogenesis. Activation of GPR3 by DPI results in a rapid recruitment of β-arrestin2 to the plasma membrane, formation of β-arrestin2-GAPDH-PKM2 super complex, greatly increased enzymatic activities of GAPDH and PKM2, and therefore a rapid increase in glycolytic activities. DPI stimulation also results in the formation of PKM2 dimers, translocation of PKM2 from the cytosol to the nucleus, transactivation of c-Myc, and transcription of glycolytic genes, leading to a sustained increase in glycolysis. In mice, DPI inhibits HFD-induced obesity and liver pathogenesis by enhancing glycolysis and suppressing inflammatory response of Kupffer cells in a PKM2-dependent manner. In patients with non-alcoholic fatty liver disease (NAFLD), single cell RNA sequencing identifies a population of disease-associated macrophages that exhibit reduced expression of glycolytic genes but increased expression of inflammatory genes. DPI stimulates glycolysis and suppresses inflammatory responses of Kupffer cells from NAFLD patients. These findings identify GPR3-β-arrestin2-PKM2 signaling as a critical pathway for metabolic reprogramming of Kupffer cells and activation of this pathway as a potential approach to inhibit the development of obesity and NAFLD.

Non-alcoholic fatty liver disease (NAFLD) is the most common liver disorder globally and is induced by fat deposition in the liver. NAFLD progresses through a series of stages: from simple steatosis to non-alcoholic steatohepatitis (NASH) to cirrhosis. Although the disease pathogenesis is not well understood, development of NAFLD is highly correlated with obesity and diabetes, and pathogenetically associated with lipid accumulation, inflammation, injury and fibrosis in the liver. As NFLAD is also a metabolic disorder, mechanisms that link metabolism to inflammation offers insights into the pathogenesis and help to identify targets for therapeutic development.

Kupffer cells (KCs) are the resident macrophages in the liver and the most abundant tissue macrophages in the body. They play a key role in detoxification, pathogen removal and tissue repair and homeostasis, but they can also contribute to the pathogenesis of liver diseases, including NAFLD, as they are involved in the initiation and progression of inflammation and tissue injury. In response to local stimuli, KCs regulate both metabolic and immune functions in the homeostatic liver. Lipids and other metabolites have been shown to not only regulate the expression of genes associated with immune response in human macrophages, but also modulate the activation of KCs in models of fatty liver disease and steatohepatitis. Disease-associated macrophages (DAMs) have been identified by single cell RNA sequencing (scRNAseq) in livers from patients with advanced NAFLD (NASH and cirrhosis) and from mouse models of NASH. DAMs exhibit altered expression of pathways associated with not only inflammation but also metabolism, suggesting that reprogramming dysfunctional macrophages may be a promising strategy to treat NAFLD.

G protein-coupled receptors (GPCRs) play essential roles in metabolic disorders as they serve as receptors for metabolites and fatty acids. In our screen for compounds that can reprogram macrophages, we found that diphenyleneiodonium (DPI), an agonist of GPR3, upregulates expression of genes involved in glycolysis and lipid metabolism. GPR3 is highly expressed in the brain and has been shown to play important roles in neurological processes. GPR3 is considered as a constitutively active orphan receptor that mediates sustained cAMP production in the absence of a ligand. An important mechanism that regulates GPCR signaling is desensitization, involving the receptor kinases (GRKs) and the β-arrestins. GPR3 stimulates the A3 production by recruiting the scaffold protein β-arrestin2 to regulate γ-secretase activity. Despite these progresses, little is known about the function and mechanism of GPR3 signaling in other cell types, especially in regulating metabolism.

We have investigated the effect of DPI on metabolic reprogramming of macrophages, the underlying molecular mechanisms, and physiological effect of DPI on high fat diet (HFD)-induced obesity and pathogenesis. We show: i) DPI induces a rapid switch of cellular metabolism from oxidative phosphorylation (OxPhos) to glycolysis in macrophages by stimulating the formation of β-arrestin2-GAPDH-PKM2 super complex with greatly increased enzymatic activities; ii) DPI also induces a prolonged increase in glycolytic activities by stimulating translocation of PKM2 from cytosol to nucleus, transactivation of c-Myc, and transcription of glycolytic genes; iii) DPI inhibits HFD-induced obesity and liver pathogenesis in mice by stimulating glycolysis and suppressing inflammation in KCs and in a manner that requires PKM2 expression in KCs; and iv) DPI also stimulates glycolysis and suppresses inflammation of KCs from patients with NAFLD. These findings identify that GPR3 to β-arrestin2 to PKM2 and to c-Myc signaling is a critical pathway for metabolic reprogramming of macrophages and activation of this pathway in KCs is an approach for therapeutic interventions of obesity and NAFLD.

DPI has been reported as an agonist of GPR3 and an inhibitor of NOX. Consistent with previous observation that NOX-deficiency leads to a lower cellular glycolysis, we found that p47phox^{-/-} BMDMs and inhibition of NOX activity by apocynin in macrophages lead to a significantly reduced basal level of glycolytic activity. However, DPI (50 nM) stimulated a similar level of increase in glycolysis in p47phox^{-/-} BMDMs as in wild-type BMDMs, or with or without inhibitor apocynin, showing that DPI stimulates glycolysis independent of NOX activity. In contrast, although GPR3 knockdown also reduces the basal level of glycolytic activities, DPI (50 nM) failed to stimulate any significant increase in glycolysis, suggesting that DPI stimulates glycolysis through activation of GPR3. Similarly, β-arrestin2 and PKM2 are required for mediating the effect of DPI on glycolysis as knockout of these genes in BMDMs abolishes DPI-induced glycolysis. The difference between β-arrestin2 and PKM2 is that the former is required for maintaining a threshold level of basal glycolytic activity while the latter is not required. These genetic analyses identify a signaling pathway involving GPR3, β-arrestin2 and PKM2 in mediating the effect of DPI on glycolysis as well as NOX, GPR3 and β-arrestin2 in maintaining a threshold level of basal cellular glycolysis. As SIP, a putative endogenous ligand of GPR3, also induces a significant increase in glycolysis in macrophages, the identified pathway likely functions in metabolic reprogramming in response to endogenous ligands.

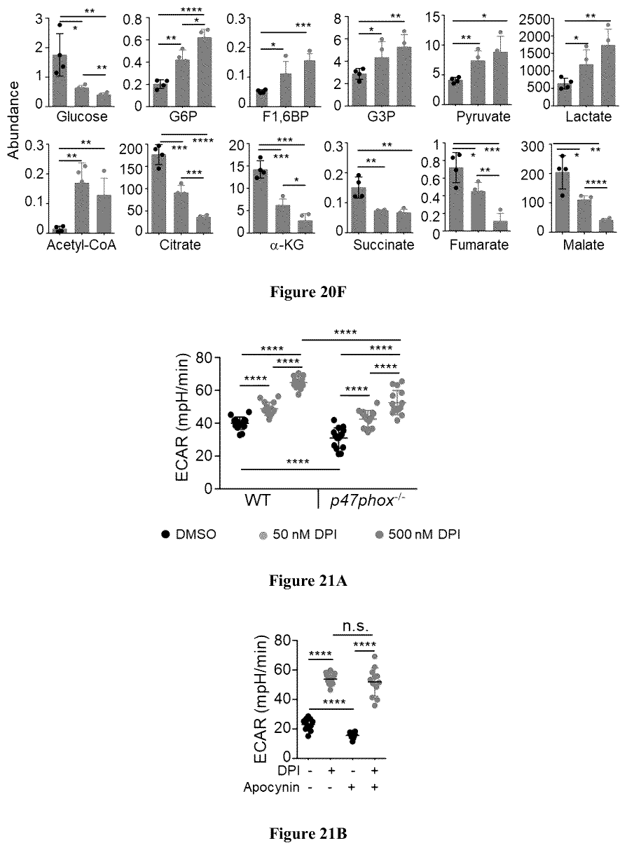

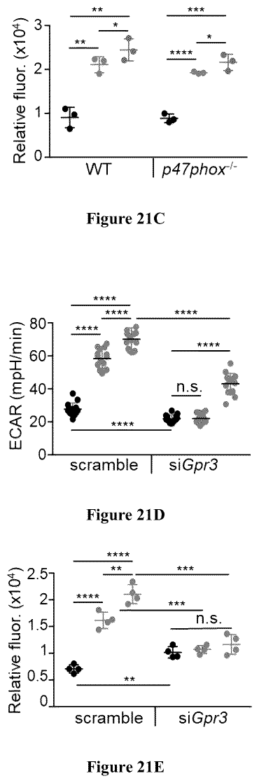

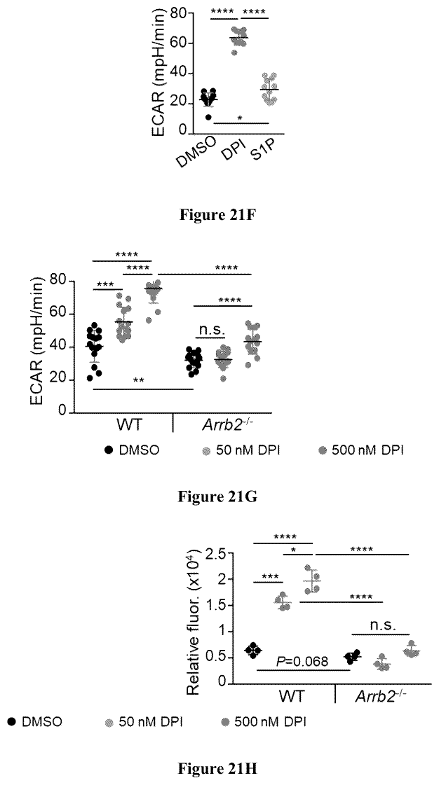

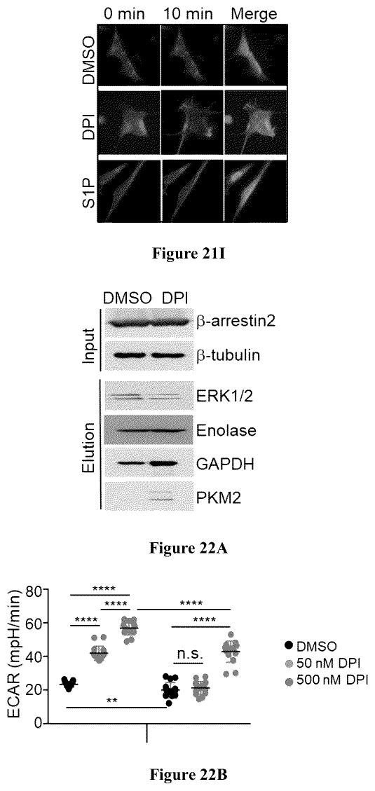

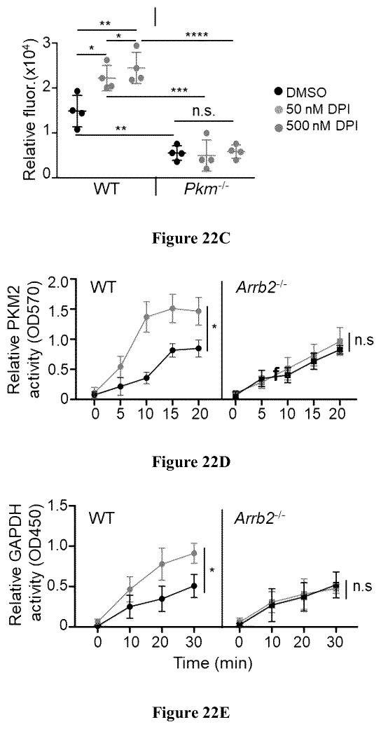

Consistent with a critical role of β-arrestin2 in GPCR signaling by functioning as a scaffold protein, we show that activation of GPR3 by DPI leads to a rapid recruitment of β-arrestin2 to the plasma membrane (FIG. 21I), presumably to GPR3. Biochemically, we further show that activation of GPR3 by DPI results in the formation of glycolytic enzyme super complex, including β-arrestin2, enolase, GAPDH and PKM2 (FIG. 22A), in an ERK1/2-dependent manner (FIG. 30). The greatly increased enzymatic activities of GAPDH and PKM2 provides a biochemical basis for the rapid increase of glycolytic activities following DPI treatment.

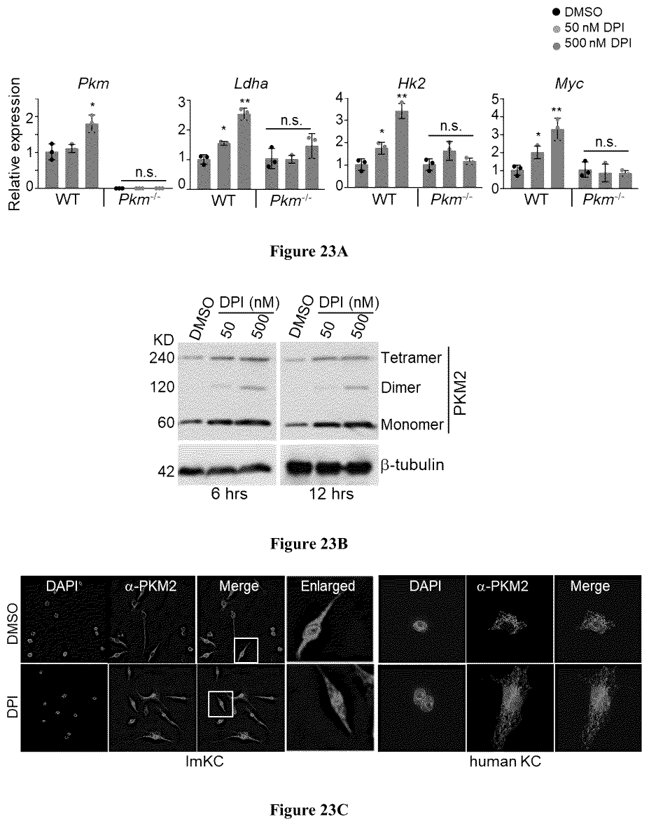

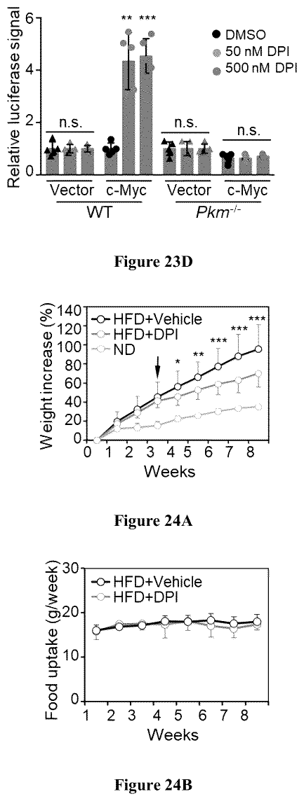

We found that activation of GPR3 by DPI also promotes the formation of PKM2 dimers in an ERK1/2-dependent manner (FIGS. 23B and 30A). ERK1/2-dependent formation of PKM2 dimers is known to translocate from the cytosol to the nucleus and activate transcription of glycolytic genes. Indeed, DPI stimulates PKM2 translocation into the nucleus in both ImKCs and primary human KCs and c-Myc transcription in an PKM2-dependent manner (FIG. 23C). c-Myc is known to directly activate almost all glycolytic genes through binding the classical E-box sequence. Besides an increased level of transcription, we also show that DPI stimulates c-Myc transactivation activities by reporter gene assay in ImKCs (FIG. 23D). These findings provide a molecular mechanism by which DPI stimulates a sustained increase of glycolytic activities in macrophages.

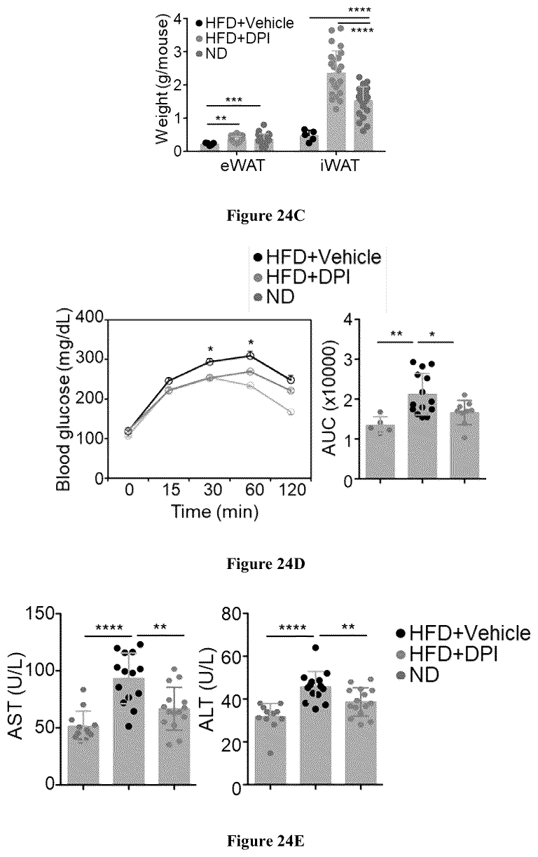

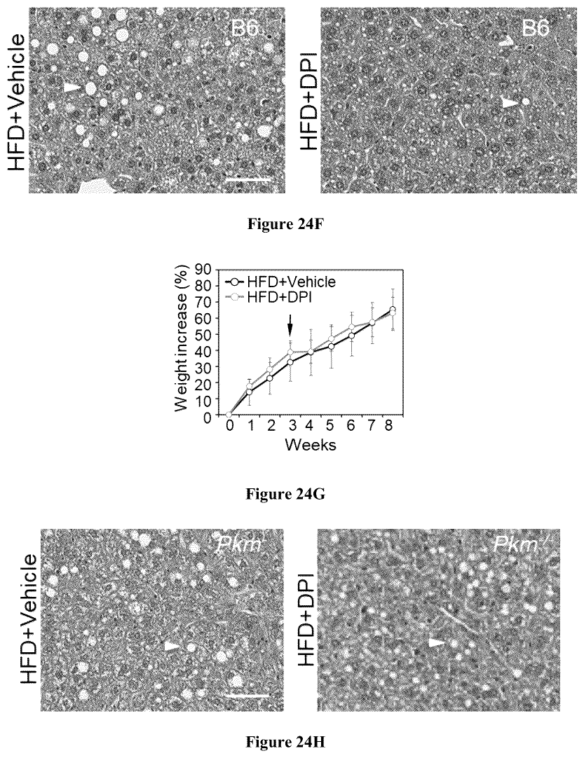

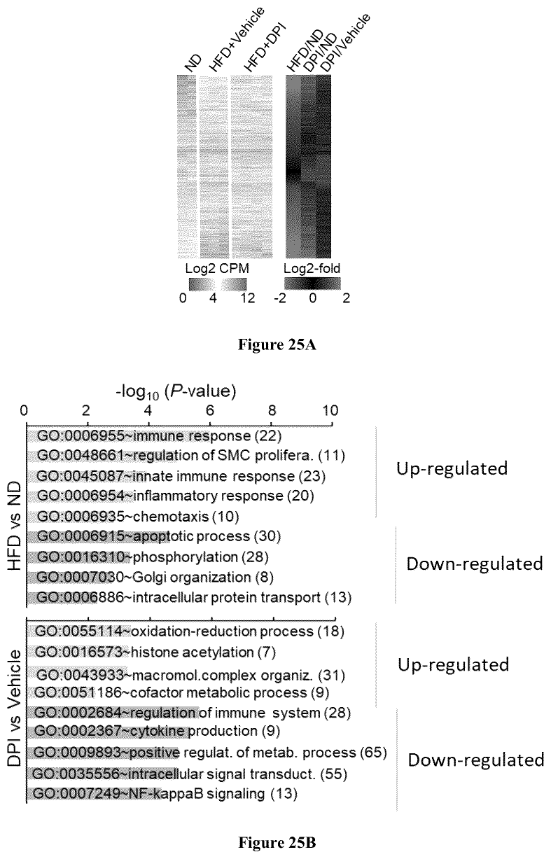

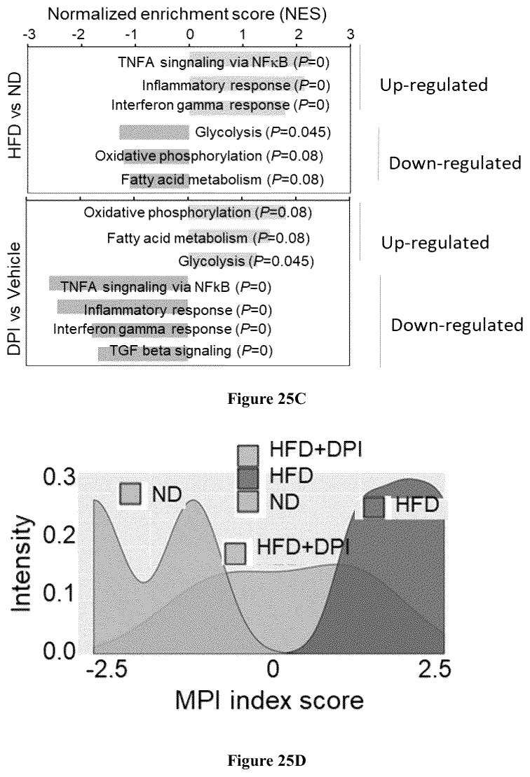

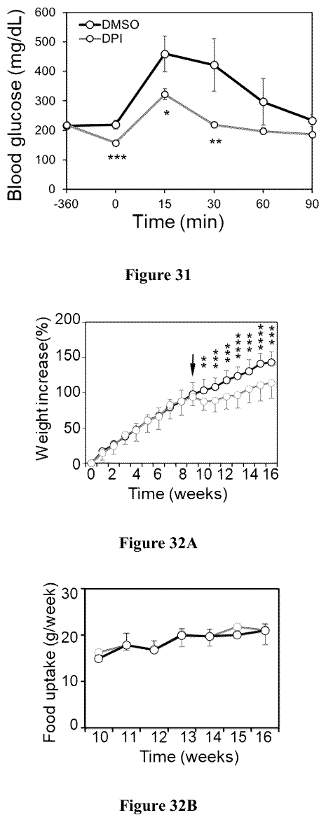

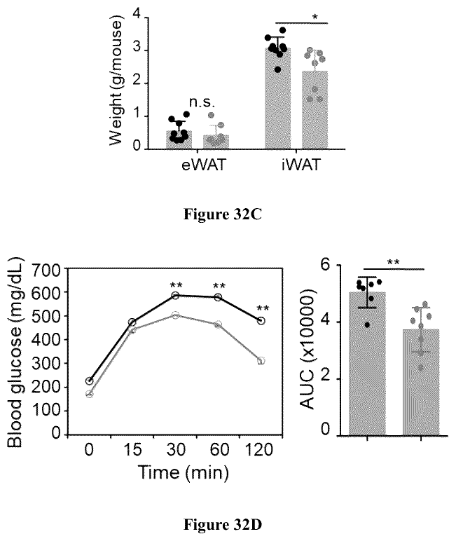

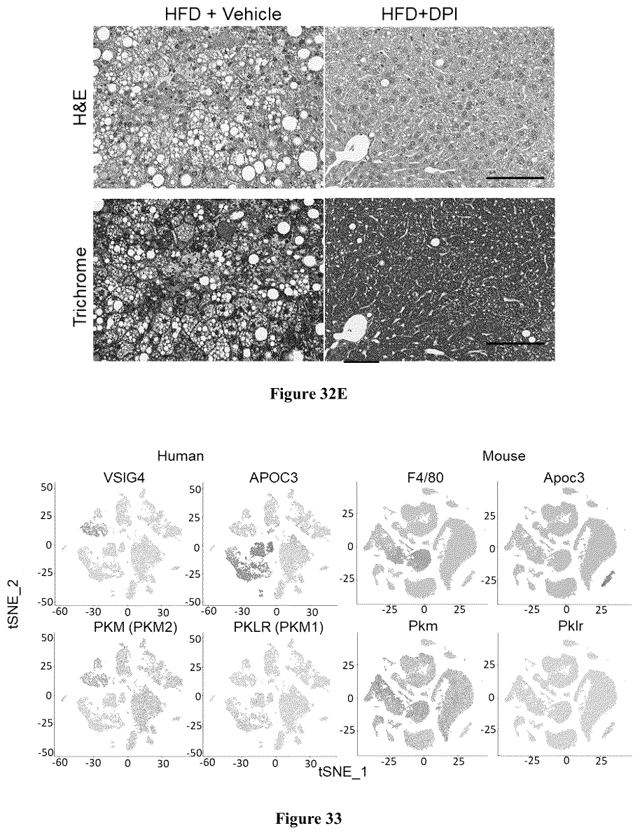

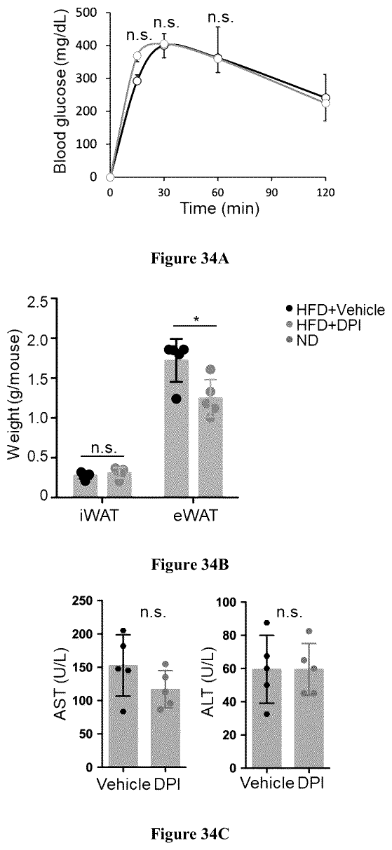

Consistent with the increased glucose consumption through elevated glycolysis, DPI has profound effects on glucose metabolism and on HFD-induced weight gain, lipid deposition and fibrosis in the liver at the organismal level. DPI confers a better glucose tolerance in mice under normal conditions (FIG. 31). DPI significantly inhibits HFD-induced weight gains without affecting feed intake (FIGS. 24 and 32). Impressively, DPI treatment of obese mice on HFD every 5 days is able to almost completely eliminate lipid droplet accumulation and fibrosis in the liver (FIGS. 24F and 32E), suggesting that DPI's effect on liver pathologies is not completely dependent on body weight reduction. Supporting this notion, KCs from HFD-fed mice with or without DPI treatment differ dramatically in expression of glycolytic and inflammatory genes (FIG. 25). DPI greatly stimulates expression of genes in glycolysis pathway but suppresses expression of inflammatory genes, showing the effect on liver pathologies is likely a result of both increased glycolysis (and therefore reduced lipid accumulation) and reduced inflammation (fibrosis). Remarkably, knockout of PKM2 specifically in KCs in mice abolishes the effect of DPI on HFD-induced obesity and liver pathogenesis (FIG. 24G-24H), suggesting that metabolic reprogramming of KCs alone is sufficient to protect from obesity and liver pathogenesis.

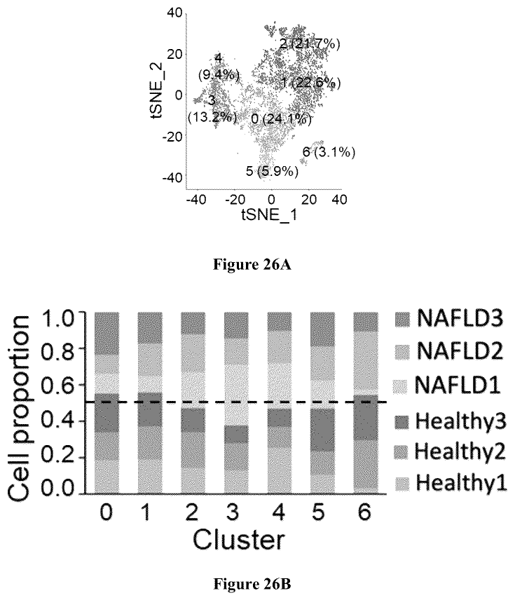

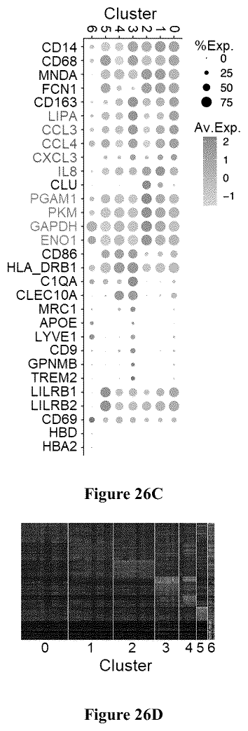

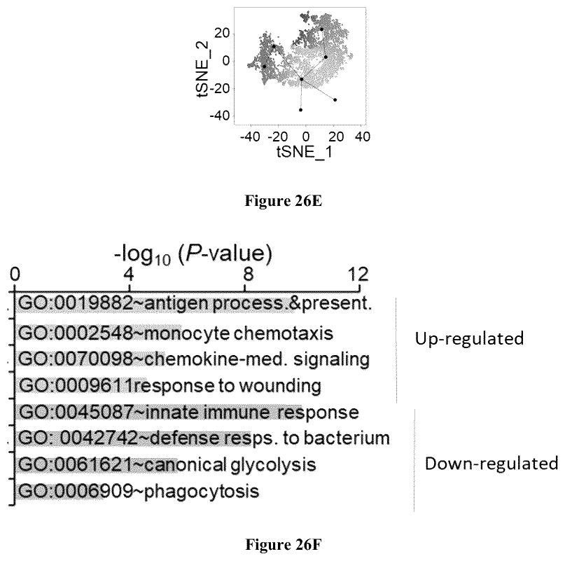

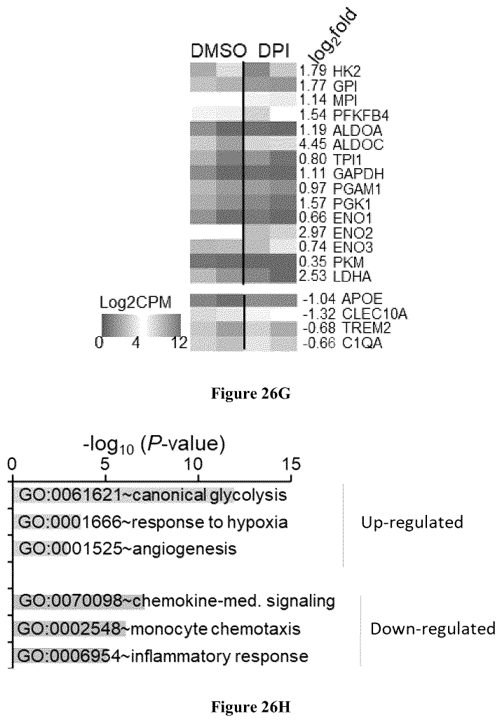

Finally, we show the presence of DAMs in the liver of NAFLD patients, which share the same phenotype, including expression of TREM2, CD9, GPNMB, MHCII (HLA-DRB1), C1QA and CLEC10A, as those found in the livers of patients with NASH and cirrhosis. As similar DAMs have been observed in various tissues with diverse pathologies, such as HFD-induced NASH in mice, scar tissues, Alzheimer's disease, and lung fibrosis, DAMs from different diseases may share a common gene expression signature. Our scRNAseq shows that DAMs are inhibited in glycolysis but increased in inflammation as suggested by downregulation of glycolytic genes and upregulation of inflammatory genes (FIG. 26). Importantly, KCs, including DAMs, from NAFLD patients respond to DPI by upregulating the transcription of glycolytic genes and downregulating the transcription of inflammatory genes (FIG. 26G-26H). As such, reprogramming macrophage metabolism, such as by DPI, is a promising therapeutic approach to treat diverse metabolic diseases.

In one aspect, described herein is a method of identifying a modulator of macrophage activation. The method comprises contacting a primary macrophage cell with a candidate agent; monitoring or photographing the morphology of the cell contacted with the candidate agent; and optionally comparing the cell's morphology in the presence of the candidate agent with the cell's morphology in the absence of the candidate agent; wherein a change in morphology in the presence of the candidate agent is indicative of modulation of macrophage activation.

In another aspect, described herein is a method of treating cancer, fibrosis, or an infectious disease. The method comprises administering to a subject in need thereof an effective amount of a modulator of macrophage activation; wherein the modulator changes the morphology of a macrophage cell from elongated shape to round shape.

In one aspect, described herein is a method of treating an inflammatory disease, a metabolic disease, an autoimmune disease, or a neurodegenerative disease. The method comprises administering to a subject in need thereof an effective amount of a modulator of macrophage activation; wherein the modulator changes the morphology of a macrophage cell from round shape to elongated shape.

In another aspect, described herein is a method of treating cancer, fibrosis, or an infectious disease. The method comprises administering to a subject in need thereof an effective amount of a modulator of macrophage activation; wherein the modulator activates a serotonin transporter or receptor, a histamine transporter or receptor, a dopamine transporter or receptor, an adrenoceptor, VEGF, EGF and/or leptin.

In one aspect, described herein is a method of treating an inflammatory disease, a metabolic disease, an autoimmune disease, or a neurodegenerative disease. The method comprises administering to a subject in need thereof an effective amount of a modulator of macrophage activation; wherein the modulator inhibits a serotonin transporter or receptor, a histamine transporter or receptor, a dopamine transporter or receptor, an adrenoceptor, VEGF, EGF and/or leptin.

In another aspect, described herein is a method of treating an inflammatory disease, a metabolic disease, an autoimmune disease, or a neurodegenerative disease. The method comprises administering to a subject in need thereof an effective amount of diphenyleneiodonium (DPI).

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

“Adjuvant” or “Adjuvant therapy” broadly refers to an agent that affects an immunological or physiological response in a patient or subject. For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given ligand) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as VH) and a heavy chain constant region. Each light chain includes a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)2, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

“Immunotherapy” is treatment that uses a subject's immune system to treat cancer and includes, for example, checkpoint inhibitors, cancer vaccines, cytokines, cell therapy, CAR-T cells, and dendritic cell therapy.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

### Screening Assays

The present disclosure provides methods of identifying a modulator of macrophage activation, comprising contacting a primary macrophage cell with a candidate agent; monitoring or photographing the morphology of the cell contacted with the candidate agent; and optionally comparing the cell's morphology in the presence of the candidate agent with the cell's morphology in the absence of the candidate agent; wherein a change in morphology in the presence of the candidate agent is indicative of modulation of macrophage activation.

As used herein, the term “test compound” or “candidate agent” refers to an agent or collection of agents (e.g., compounds) that are to be screened for their ability to have an effect on the cell. Test compounds can include a wide variety of different compounds, including chemical compounds, mixtures of chemical compounds, e.g., polysaccharides, small organic or inorganic molecules (e.g., molecules having a molecular weight less than 2000 Daltons, less than 1500 Dalton, less than 1000 Daltons, or less than 500 Daltons), biological macromolecules, e.g., peptides, proteins, peptide analogs, and analogs and derivatives thereof, peptidomimetics, nucleic acids, nucleic acid analogs and derivatives, an extract made from biological materials such as bacteria, plants, fungi, or animal cells or tissues, naturally occurring or synthetic compositions.

Depending upon the particular embodiment being practiced, the test compounds can be provided free in solution, or can be attached to a carrier, or a solid support, e.g., beads. A number of suitable solid supports can be employed for immobilization of the test compounds. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminemethylvinylether maleic acid copolymer, glass beads, amino acid copolymer, ethylene-maleic acid copolymer, nylon, silk, etc. Additionally, for the methods described herein, test compounds can be screened individually, or in groups. Group screening is particularly useful where hit rates for effective test compounds are expected to be low such that one would not expect more than one positive result for a given group.

A number of small molecule libraries are known in the art and commercially available. These small molecule libraries can be screened using the screening methods described herein. A chemical library or compound library is a collection of stored chemicals that can be used in conjunction with the methods described herein to screen candidate agents for a particular effect. A chemical library comprises information regarding the chemical structure, purity, quantity, and physiochemical characteristics of each compound. Compound libraries can be obtained commercially, for example, from Enzo Life Sciences™, Aurora Fine Chemicals™, Exclusive Chemistry Ltd.™, ChemDiv, ChemBridge™, TimTec Inc.™, AsisChem™, and Princeton Biomolecular Research™, among others.

Without limitation, the compounds can be tested at any concentration that can exert an effect on the cells relative to a control over an appropriate time period. In some embodiments, compounds are tested at concentrations in the range of about 0.01 nM to about 100 nM, about 0.1 nM to about 500 microM, about 0.1 microM to about 20 microM, about 0.1 microM to about 10 microM, or about 0.1 microM to about 5 microM.

The compound screening assay can be used in a high throughput screen. High throughput screening is a process in which libraries of compounds are tested for a given activity. High throughput screening seeks to screen large numbers of compounds rapidly and in parallel. For example, using microtiter plates and automated assay equipment, a laboratory can perform as many as 100,000 assays per day, or more, in parallel.

The compound screening assays described herein can involve more than one measurement of the cell or reporter function (e.g., measurement of more than one parameter and/or measurement of one or more parameters at multiple points over the course of the assay). Multiple measurements can allow for following the biological activity over incubation time with the test compound. In one embodiment, the reporter function is measured at a plurality of times to allow monitoring of the effects of the test compound at different incubation times.

The screening assay can be followed by a subsequent assay to further identify whether the identified test compound has properties desirable for the intended use. For example, the screening assay can be followed by a second assay selected from the group consisting of measurement of any of: bioavailability, toxicity, or pharmacokinetics, but is not limited to these methods.

### EXAMPLES

The invention now being generally described, it will be more readily understood by reference to the following examples which are included merely for purposes of illustration of certain aspects and embodiments of the present invention, and are not intended to limit the invention.

### Example 1: Experimental Procedures

### Human Monocyte-Derived Macrophages and Cell Lines

Human peripheral blood mononuclear cells (PBMCs) were isolated from fresh blood (Research Blood Components LLC.) by density gradient centrifugation with Ficoll-Paque Plus (GE healthcare) and LeucoSep™ (Greiner Bio-one). Human monocytes were purified from PBMC using the EasySep™ human monocyte isolation kit (Stemcell Technology) according to the manufacture's protocol. For in vitro differentiation of monocytes into human macrophages (M0, primary macrophage), isolated monocytes were cultured in complete RMPI1640 supplemented with 10% FCS (Gibco), 2 mM L-glutamine (Corning) and 1% PenStrep solution (Corning) in the presence of 50 ng/mL recombinant human M-CSF (Peprotech) for 7 days. Tumor cell line B16F10 were purchased from ATCC and cultured in complete DMEM supplemented with 10% FCS, 1% PenStrep solution and 2 mM L-glutamine. Luciferase-expressing human lymphoma B cell line (GMB) were described in Roghanian et al. Cancer Immunol Res (2019) and cultured in complete RPMI 1640 containing 10% FCS, 2 mM L-glutamine, 0.55 mM 2-mercaptoethanol (Gibco), 1% non-essential amino acids (Lonza), 1 mM sodium pyruvate (Cellgro) and 1% PenStrep solution.

### High Throughput Compound Screening, High-Content Microscope and Image Analysis

Based on the shape difference of M1 (round) and M2 (elongated) differentiated macrophages, we developed a high throughput method to screen compounds which could modulate macrophage polarization. Human M0 primary macrophages differentiated from monocytes in vitro were seeded using a Multidrop Combi dispenser (Thermo Scientific) at a density of 5,000 cells/well in 50 μL complete RPMI in the presence of 10 ng/mL M-CSF into optical 384-well plates (Cat. 393562, BD Falcon) and cultured for 16 hrs for cell recovery. Around 20% of macrophages in this stage (M0) are elongated. Cells were treated with a library of over 4000 individual compounds or drugs at the final concentration of 20 μM using the CyBi-Well simultaneous pipettor (CyBio). The screening compound library composes of the 2066 bioactive compounds, 320 FDA approved drugs, 440 oncological drugs and 1280 natural compounds from the center for the development of therapeutics in Broad Institute at MIT. After 24 hr incubation, supernatants were removed using the microplate washer (Bioteck) and cells were fixed by adding 50 μL 16% paraformaldehyde (Thermo Scientific) with the dispenser for 20 minutes. Cells were then washed with 50 μL 1×PBS twice and incubated for 20 minutes with NucBlue and AF746 Phalloidin (Invitrogen) to stain nucleus and cytoskeleton. Cells were then washed with 50 μL 1×PBS twice and maintained in PBS for the image acquirement. Plates were read in the Opera Phenix high content screening system (PerkinElmer) to photograph cells using 20× objective in 2 fluorescent channels (Blue and FarRed). A total of 6 different fields in each well and an average of 1,000 cells were imaged per well. CellProfiler was used to identify each cell by overlapping signals from its nucleus and cytoskeleton, and calculate the eccentricity as the parameter to measure the cell morphology. The Z-score was calculated by T-test to measure the difference of cell morphology between each treatment and control. For each row of the 384-well plate, total 4 wells with first and last two columns treated with the same concentration of DMSO were combined as the control for the other 20 treatment wells in that row. In the meantime, classic M1 and M2 stimuli were added to generate the gold-standard Z-score cutoffs with M1 or M2 activation. Classic M1 stimuli include LPS (100 ng/mL), IFNγ (50 ng/mL, Peprotech), TNFα (50 ng/mL, Peprotech), or IFNγ plus TNFα. Classic M2 stimuli include IL-10 (10 ng/mL, Peprotech), IL-4 (10 ng/mL, Peprotech), or IL-13 (5 ng/mL, Peprotech). The gold-standard Z-scores were used as the cutoffs to identify potent compounds to activate macrophage into M1 or M2 state.

To further screen to compounds which could reactivate or reprogram differentiated macrophages, potent 127 M1-activating and 180 M2-activating compounds from the first-round screening were cherry-picked up. Human macrophages were seeded into optical 384-well plates. Sixteen hours later, medium in M1 plates were replaced by M1 differentiating medium (complete RPMI with 50 ng/mL IFNγ and 50 ng/mL TNFα) and medium in M2 plates by M2 differentiating medium (complete RPMI with 5 ng/mL IL-4 and 5 ng/mL IL-13). After 24 hrs cell differentiation, M1 plates (M1 macrophages) and M2 plates (M2 macrophages) were treated with M2-activating compounds and M1-activating compounds respectively for 24 hrs. Two independent experiments were performed with or without replacing differentiating medium right before treatment. Cell imaging and analysis were performed as indicated above.

### Compound Target and Pathway Analysis

The identified compounds were classified based on the database from the International Union of Basic and Clinical Pharmacology (IUPHAR) (guidetopharmacology.org). The protein targets of the compounds were text-mined based on the target databases of UPHAR and DrugBank (drugbank.ca). The pathway enrichment analysis of protein targets of compounds was based on the WikiPathways.

### Mice, Antibodies and Flow Cytometry

B6 mice were purchased from the Jackson Laboratory and maintained in the animal facility at the Massachusetts Institute of Technology (MIT). NSG mice were purchased from the Jackson Laboratory and maintained under specific pathogen-free conditions in the animal facilities at MIT. All animal studies and procedures were approved by the Massachusetts Institute of Technology's Committee for Animal Care. Flow cytometry antibodies specific for mouse CD11b (M1/70), F4/80 (BM8), MHC-II (M5/114.15.2), Ly6C (HK1.4), Ly6G (1A8), Gr-1 (RB6-8C5), CD80 (16-10A1), CD86 (GL-1), CD163 (S15049I), CD206 (C068C2), IFNγ (XMG1.2) and TNFα (MP6-XT22) were from Biolegend (USA) and iNOS (CXNFT) as from eBioscience (USA). Flow cytometry antibodies specific for human CD80 (2D10), CD86 (BU63), CD163 (GHI/61) and CD206 (15-2) were fopm Biolegend (USA) and iNOS (4E5) was from Novus Biologicals (USA). Antibody ARG1 (AlexF5) specific for both human and mouse was from eBioscience (USA). B16F10 melanoma specific antibody TA99 for in vivo study was prepared as described. Single cell preparation from different organs, staining of cells with fluorophore-conjugated antibodies and analysis of the stained cells using flow cytometry are as described. Briefly, cells in single cell suspension were incubated with specific antibodies at 4° C. for 20 minutes, washed twice, and resuspended in FACS buffer containing either DAPI. Cells were fixed and permeabilized with Cyto-Fast Fix/Perm buffer set (Biolegend) for intracellular staining according to the manufacture's protocol. Samples were stimulated by the cell stimulation cocktail (eBioscience) for 4 hrs and then fixed/permeabilized for intracellular staining. Cells were run on BD-LSRII, collecting 20,000 to 100,000 live cells per sample. The data were analyzed by FlowJo.

### Mouse Tumor Model and Treatment

For the melanoma model, an inoculum of 1×10⁶ B16F10 tumor cells was injected subcutaneously on the flank of 8- to 10-week-old male B6 mice in 100 L sterile PBS. Six days following tumor inoculation, mice were randomized into 4 treatment groups including control (PBS or DMSO), tumor-targeting antibody TA99, compound, compound plus TA99. TA99 was administered at 100 g per dose intraperitoneally (I.P.). The compound was administrated at the indicated dosage by either I.P. or paratumor injection subcutaneously (S.C.). All mice were dosed at day 6 and day 12 post tumor inoculation for a total of 2 treatments. Tumor size was measured as an area (longest dimension×perpendicular dimension) at day 6, day 12 and day 18 post tumor inoculation. Mice were euthanized for analysis at day 18 post tumor inoculation. For the lymphoma model, 1×10⁷ GMB cells were injected through tail intravenously in 100 μL sterile PBS into 10- to 12-week-old male NSG mice. Mice were treated two weeks post tumor cell engraftment. Tumor-targeting antibody Rituxumab (InvivoGen) was administered at 10 mg/kg intraperitoneally. The compound was administrated I.P. at the indicated dosage. All mice were dosed at week 2 and week 3 post tumor injection for a total of 2 treatments. Tumor growth and spread was visualized using an IVIS Spectrum-bioluminescent imaging system (PerkinElmer) at week 2, week 3 and week 4 post tumor injection. Mice were euthanized for analysis at week 4 post tumor inoculation.

### Histopathology and Immunochemical Staining

Mice were euthanized and tumor tissues were isolated and fixed with 10% neutral-buffered formalin solution (Sigma-Aldrich) for 24 hours. The tissues were processed with Tissue Processor (Leica Microsystems) and embedded in paraffin. Sections were cut at 5 m thickness, mounted on polylysine-coated slides (Thermo Fisher Scientific), de-waxed, rehydrated, and processed for hematoxylin and eosin (H&E) staining according to a standard protocol. For immunochemical staining, antigen retrieval was carried out by either microwaving the slides in 0.01 M sodium citric acid buffer (pH 6.0) for 30 min. Sections were then immersed for 1 hour in blocking buffer (3% BSA, 0.2% Triton X-100 in PBS), then incubated in primary antibody in blocking buffer at 4° C. overnight, followed by incubation with secondary antibody conjugated HRP at 4° C. for 1 hour. All lung stained sections were scanned with a high-resolution Leica Aperio Slide Scanner. Images were analyzed by WebScope software.

### Mouse Bone Marrow-Derived Macrophages and Tumor-Associated Macrophages

Mouse bone marrow-derived macrophages (mBMM) were prepared as described previously⁵⁴. Briefly, fresh bone marrow cells were isolated from B6 mice. Cells were plated into 6-well plate with 1×10⁶/mL in complete RPMI with 2-mercaptoethanol and cultured for 6 days with fresh medium change every 2 days. mBMMs were differentiated to resemble TAMs in the presence of 10 ng/mL mIL-4 and mIL-13 (Peprotech) or 25 mM lactate acid for 24 hrs or tumor conditioned medium (CM). To prepare CM, 70% confluent B16F10 cultured were replaced with fresh medium and the tumor medium was collected and filtered (0.2 m) 24 hrs later. The mixture of 3 volumes of tumor medium with 1 volume of complete RPMI for mBMM serves as the CM. Expression of Arg, Fizz1 and Vegfa were quantified by qPCR to assess the development of TAMs. Other genes of Tnf, Il1b, Nos2, Cxcl2, Ccl5, Ym1 and Tgfb serve as macrophage activating markers. To assay the tumor growth inhibition, mBMMs (10,000 cells per well in 96 well plate) were treated with thiostrepton for 24 hrs and then cocultured with equal number of B16 melanoma cells in fresh complete RPMI for 12 hrs. The conditioned medium treated or not treated with thiostrepton were collected and filtered. The numbers of B16 melanoma cells were cultured for 12 hrs with conditioned medium or conditioned medium heated at 95° C. for 5 min. Tumor cells were quantified by flow cytometry to determine the macrophage-dependent killing function.

### RNA Isolation, RNA Sequencing and Data Analysis

RNAs were extracted with RNeasy MiniElute kit (Qiagen), converted into cDNA and sequenced using Next-Generation Sequencing (Illumina). RNA-seq data was aligned to the mouse genome (version mm10) and raw counts of each genes of each sample were calculated with bowtie2 2.2.3 and RSEM 1.2.15. Differential expression analysis was performed using the program edgeR at P<0.05 with a 2 fold-change. The gene expression level across different samples was normalized and quantified using the function of cpm. Differentially expressed genes were annotated using online functional enrichment analysis tool DAVID (http://david.ncifcrf.gov/). Gene set enrichment analysis were performed with GSEA with FDR q-value<0.05. The heatmap figure was visualized with MeV. To quantify the levels of RNA transcripts, total RNA was extracted from various cells and reverse transcribed by TaqManÂ® Reverse Transcription Reagents Kit (ABI Catalog No. N8080234), followed by amplification with Sybr Green Master Mix (Roche Catalog No. 04707516001) with specific primers (Table 4) and detected by Roche LightCycler 480. The Ct values were normalized with housekeeping gene GAPDH for comparison.

Table 4 shows primers for qPCR.

TABLE 4
shows primers for qPCR.

| mouse Primer | Sequence | SEQ ID NO: |
|---|---|---|
| Arg1-F | CATTGGCTTGCGAGACGTAGAC | 1 |
| Arg1-R | GCTGAAGGTCTCTTCCATCACC | 2 |
| Fizz1-F | CAAGGAACTTCTTGCCAATCCAG | 3 |
| Fizz1-R | CCAAGATCCACAGGCAAAGCCA | 4 |
| Vegfa-F | CTGCTGTAACGATGAAGCCCTG | 5 |
| Vegfa-R | GCTGTAGGAAGCTCATCTCTCC | 6 |
| Ym1-F | TACTCACTTCCACAGGAGCAGG | 7 |
| Ym1-R | CTCCAGTGTAGCCATCCTTAGG | 8 |
| Tgfb-F | TGATACGCCTGAGTGGCTGTCT | 9 |
| Tgfb-R | CACAAGAGCAGTGAGCGCTGAA | 10 |
| Tnf-F | GGTGCCTATGTCTCAGCCTCTT | 11 |
| Tnf-R | GCCATAGAACTGATGAGAGGGAG | 12 |
| Il1b-F | ACGGCTGAGTTTCAGTGAGACC | 13 |
| Il1b-R | CACTCTGGTAGGTGTAAGGTGC | 14 |
| Ccl2-F | GCTACAAGAGGATCACCAGCAG | 15 |
| Ccl2-R | GTCTGGACCCATTCCTTCTTGG | 16 |
| Ccl5-F | CCTGCTGCTTTGCCTACCTCTC | 17 |
| Ccl5-R | ACACACTTGGCGGTTCCTTCGA | 18 |
| Cxcl2-F | CATCCAGAGCTTGAGTGTGACG | 19 |
| Cxcl2-R | GGCTTCAGGGTCAAGGCAAACT | 20 |
| Gapdh-F | AGTATGACTCCACTCACGGC | 21 |
| Gapdh-R | GTTCACACCCATCACAAACA | 22 |
| Nos2_F | GAGACAGGGAAGTCTGAAGCAC | 23 |
| Nos2_w | CCAGCAGTAGTTGCTCCTCTTC | 24 |
| human Primer | Sequence | |
| GAPDH-F | GTCTCCTCTGACTTCAACAGCG | 25 |
| GAPDH-R | ACCACCCTGTTGCTGTAGCCAA | 26 |
| TNF-F | CTCTTCTGCCTGCTGCACTTTG | 27 |
| TNF-R | ATGGGCTACAGGCTTGTCACTC | 28 |
| IL1B-F | CCACAGACCTTCCAGGAGAATG | 29 |
| IL1B-R | GTGCAGTTCAGTGATCGTACAGG | 30 |
| CXCL2-F | GGCAGAAAGCTTGTCTCAACCC | 31 |
| CXCL2-R | CTCCTTCAGGAACAGCCACCAA | 32 |
| IL10-F | TCTCCGAGATGCCTTCAGCAGA | 33 |
| IL10-R | TCAGACAAGGCTTGGCAACCCA | 34 |
| CD86-F | TCATTCCCTGATGTTACGAGC | 35 |
| CD86-R | TCTTCCCTCTCCATTGTGTTG | 36 |
| CD163-F | GTGTGATGACTCTTGGGACTTG | 37 |
| CD163-R | AGGATGACTGACGGGATGAG | 38 |
| CD206-F | GACTGATAAGTGGAGGGTGAGG | 39 |
| CD206-R | CCAGAGAGGAACCCATTCG | 40 |

### Macrophage Activation Network Induced by Compounds

To determine the central hubs of all stimulation conditions by compounds (refer to FIG. 4) reflecting the core macrophage activation network, transcriptional interactions between genes were first determined by ARACNe based on the perturbed transcriptional profiles of 34 compounds as well as IFNγ and IL4 controls. The 12549 unique present genes were taken into calculation of mutual information with p-value less than 1e-7. The threshold of the data processing inequality (DPI) theorem from information theory used by ARACNe was set to 0.1 to detect total 400,165 regulatory interactions in the core macrophage activation network. GO enrichment analysis and enrichment map of top 10% central hubs (1255 genes) was performed by BiNGO. The network was visualized by Cytoscape.

### Statistic Methods

Statistical significance was determined with the two-tailed unpaired or paired Student's t-test. The FDRs were computed with q=p*n/i, (p=P value, n=total number of tests, i=sorted rank of P value).

### Data Availability

Raw RNAseq are deposited in the database of Gene Expression Omnibus (GEO) with accession ID: GSE14992 and GSE155551.

### Example 2: Phenotypic Screen of Macrophage Activation

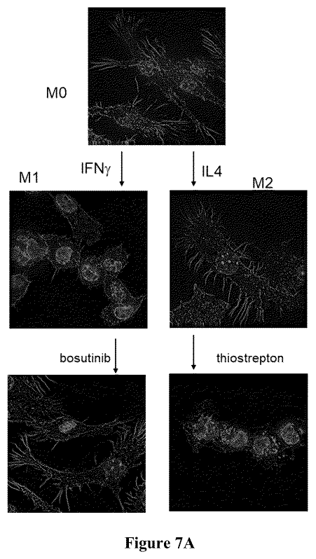

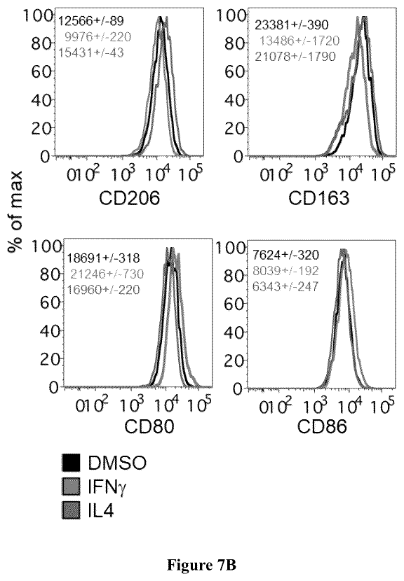

Human monocytes were isolated from peripheral blood mononuclear cells (PBMCs) and differentiated into macrophages in a 7-day culture in the presence of recombinant human M-CSF. The resulting human monocyte-derived macrophages (hMDMs) were stimulated with different known M1-activating stimuli, including lipopolysaccharide (LPS), IFNγ, TNFα, or IFNγ plus TNFα, or M2-activating cytokines, including IL-10, IL-4 or IL-13, for 24 hours. The M1-activated hMDMs were round with punctate F-actin staining whereas M2-activated hMDMs were elongated with filamentous F-actin staining (FIGS. 1A and 7A). Expression of known M1 markers including CD80 and CD86 were up-regulated by IFNγ and suppressed by IL-4 while M2 markers CD206 and CD163 were up-regulated by IL-4 and suppressed by IFNγ (FIG. 7B). The Z-score for each stimulus was calculated to index its activation ability from the distributions of cell shapes between treated wells and untreated wells by T-test of an average of 1000 cells per well. The M1-activated hMDMs had an average of Z-score of −4 whereas the M2-activated hMDMs had an average of Z-score of 6 (FIG. 1B). M1- and M2-like human and mouse macrophages have distinct morphologies.

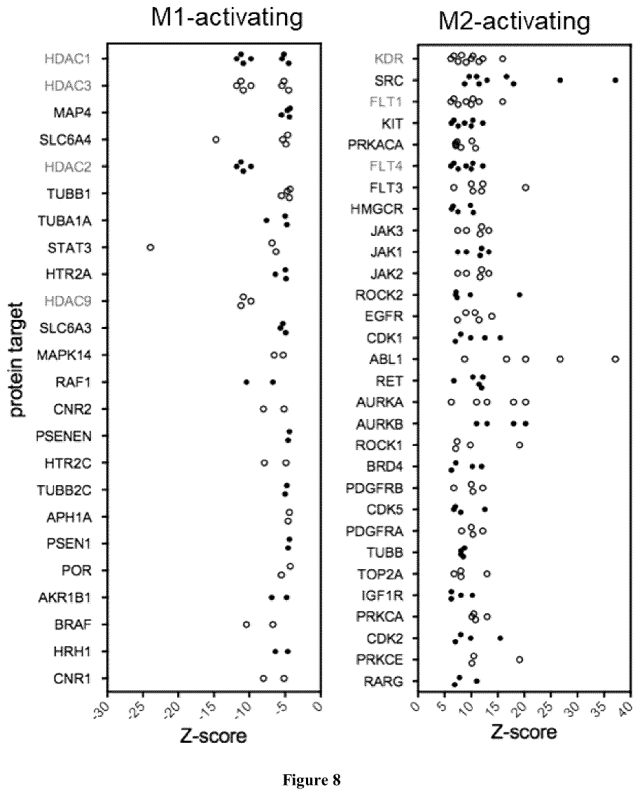

Based on the correlation between cell shape and macrophage activation, we developed a high throughput screen for compounds that activate hMDMs to either M1- or M2-like state (FIG. 1C). Human monocytes purified from four healthy donors were mixed at equal ratio and differentiated into macrophages with M-CSF. The resulting macrophages were seeded into 384-well plates and cultured overnight in the presence of M-CSF to maintain macrophages at mostly a non-activated stage. Macrophages in each well were then treated with one of 4126 compounds, including 2086 bioactive compounds, 760 FDA-approved drugs, and 1280 natural products (FIG. 1D), at a final concentration of 20 M for 24 hours. Cell images were taken by high-content scanning microscope and cell shapes were quantified by Cellprofiler (FIG. 1E). Based on Z-score cutoffs: −4 for M1-activated macrophages and 6 for M2-activated macrophages, 127 and 180 compounds were identified, respectively, to activate human macrophages toward M1-like state (referred to as M1-activating compounds) and M2-like state (referred to as M2-activating compounds) (FIG. 1F). 98 of 127 (77%) M1-activating and 166 of 180 (92%) M2-activating compounds are FDA-approved drugs (FIG. 1G). Text-mining identified 119 known protein targets for 80 of the 127 M1-activating compounds and 220 protein targets for 144 of the 180 M2-activating compounds. The targets include G-protein coupled receptors (GPCRs), enzymes, kinases, nuclear hormone receptors (NHRs), and transporters (FIG. 1G). Many targets of M1- and M2-activating compounds belong to the families of histone deacetylases and VEGF receptors, respectively (FIG. 8). Some known regulators of macrophage polarization, such as STAT3, FYN, MAP2K1 and CDKs, were rediscovered. Pathways analysis of the protein targets identified known pathways, such as IL-4, IL-1β, and TGFβ pathways, and novel pathways, such as neurotransmitter, leptin, EGF and VEGF signaling pathways, in macrophage activation (FIG. 1H and Table 1).

Table 1 shows pathway analysis of proteins targeted by identified compounds.

| Pathway | | Number | | | | |
|---|---|---|---|---|---|---|
| Name | Number | Genes | | | Number | Average |
| (Wiki) | Compounds | Pathway | Compound List | Target List | Targets | Z-value |
| GPCRs, | 13 | 261 | 2-[(4-Phenylpiperazin-1-yl) | PTGDR; CNR1; | 19 | −5.92 |
| Class A | | | methyl]-2,3-dihydroimidazo | HTR2A; HTR1A; | | |
| Rhodopsin-like | | | [1,2-c]quinazolin-5(6H)-one; | DRD2; CNR2; | | |
| | | | FTY720; Terciprazine; | HRH2; PTGIR; | | |
| | | | Diphenyleneiodonium | CYSLTR1; HTR2C; | | |
| | | | chloride; PIMOZIDE; “WIN | AGTR1; ADRA1A; | | |
| | | | 55,212-2 mesylate”; | DRD3; PTGER4; | | |
| | | | TCB2; FLUOXETINE; | GPR3; PTGER1; | | |
| | | | Alprostadil; SCH 79797 | HRH1; PTGER2; | | |
| | | | dihydrochloride; DOXEPIN | F2R | | |
| | | | HYDROCHLORIDE; FPL | | | |
| | | | 55712; CANDESARTAN | | | |
| | | | CILEXTIL | | | |
| Leptin | 12 | 78 | cucurbitacin I; Vemurafenib; | GSK3A; RAF1; | 12 | −7.54 |
| signaling | | | niclosamide; Vemurafenib; | MAPK14; IKBKG; | | |
| pathway | | | skepinone-L; SMER 3; niclosamide; | MTOR; AKT1; | | |
| | | | SB 202190; IKK 16; Dephostatin; | PTPN1; IKBKB; | | |
| | | | CHIR-99021; API-2 | GSK3B; CHUK; | | |
| | | | | STAT3; MAPK1 | | |
| B Cell | 9 | 100 | Vemurafenib; Vemurafenib; | GSK3A; RAF1; | 12 | −5.82 |
| Receptor | | | skepinone-L; SB 202190; | MAPK14; PTPN6; | | |
| Signaling | | | LFM-A13; IKK 16; Dephostatin; | IKBKG; BRAF; | | |
| Pathway | | | CHIR-99021; API-2 | AKT1; IKBKB; | | |
| | | | | GSK3B; CHUK; | | |
| | | | | MAPK1; BTK | | |
| Notch | 16 | 61 | cucurbitacin I; MGCD-0103; | PSENEN; HDAC1; | 11 | −7.78 |
| Signaling | | | MS-275; MS-275; MS-275; | MTOR; AKT1; | | |
| Pathway | | | niclosamide; SMER 3; | APH1A; GSK3B; | | |
| | | | niclosamide; CI-994; CI- | EP300; HDAC2; | | |
| | | | 994; PLUMBAGIN; MK- | PSEN1; STAT3; | | |
| | | | 0752; CHIR-99021; CI- | NCSTN | | |
| | | | 994; DAPT; API-2 | | | |
| BDNF | 15 | 146 | cucurbitacin I; | CNR1; RAF1; | 11 | −8.01 |
| signaling | | | Cantharidin; Vemurafenib; | MAPK14; NGF; | | |
| pathway | | | FTY720; Ro 08-2750; | PPP2CA; MTOR; | | |
| | | | niclosamide; Vemurafenib; | AKT1; NTRK2; | | |
| | | | skepinone-L; SMER 3; | GSK3B; STAT3; | | |
| | | | niclosamide; DEOXYGEDUNIN; | MAPK1 | | |
| | | | SB 202190; “WIN 55,212-2 | | | |
| | | | mesylate”; CHIR-99021; API-2 | | | |
| IL-4 | 12 | 56 | cucurbitacin | MAPK14; PTPN6; | 10 | −6.90 |
| Signaling | | | I; niclosamide; skepinone-L; | AKT1; IKBKB; | | |
| Pathway | | | PIMOZIDE; niclosamide; SB | EP300; CHUK; | | |
| | | | 202190; IKK | HRH1; MAPK11; | | |
| | | | 16; PLUMBAGIN; DOXEPIN | STAT3; MAPK1 | | |
| | | | HYDROCHLORIDE; Dephostatin; | | | |
| | | | CHIR-99021; API-2 | | | |
| Kit | 13 | 59 | cucurbitacin I; | RAF1; MAPK14; | 9 | −7.34 |
| receptor | | | Vemurafenib; niclosamide; | PTPN6; MTOR; | | |
| signaling | | | Vemurafenib; skepinone-L; | AKT1; EP300; | | |
| pathway | | | SMER 3; niclosamide; SB | STAT3; MAPK1; | | |
| | | | 202190; LFM-A13; | BTK | | |
| | | | PLUMBAGIN; Dephostatin; | | | |
| | | | CHIR-99021; API-2 | | | |
| MAPK | 9 | 168 | Vemurafenib; Vemurafenib; | RAF1; MAPK14; | 9 | −5.75 |
| Signaling | | | skepinone-L; SB 202190; IKK16; | PAK1; IKBKG; | | |
| Pathway | | | CMPD-1; CHIR-99021; API-2; | BRAF; AKT1; | | |
| | | | PF-3758309 | IKBKB; MAPKAPK2; | | |
| | | | | MAPK1 | | |
| Insulin | 8 | 160 | Vemurafenib; Vemurafenib; | GSK3A; RAF1; | 9 | −5.91 |
| Signaling | | | skepinone-L; SB 202190; IKK | MAPK14; AKT1; | | |
| | | | 16; Dephostatin; CHIR- | PTPN1; IKBKB; | | |
| | | | 99021; API-2 | GSK3B; MAPK11; | | |
| | | | | MAPK1 | | |
| Focal | 6 | 191 | cytochalasin B; | RAF1; PAK1; | 9 | −9.11 |
| Adhesion | | | Vemurafenib; Vemurafenib; | BRAF; AKT1; | | |
| | | | CHIR-99021; API-2; | PAK6; PAK4; | | |
| | | | PF-3758309 | GSK3B; ACTG1; | | |
| | | | | MAPK1 | | |
| TGF beta | 16 | 135 | MGCD-0103; MS-275; MS-275; | HDAC1; RAF1; | 8 | −7.01 |
| Signaling | | | Vemurafenib; MS-275; | MAPK14; UCHL5; | | |
| Pathway | | | Vemurafenib; skepinone-L; | MTOR; AKT1; | | |
| | | | SMER 3; CI-994; SB | EP300; MAPK1 | | |
| | | | 202190; CI-994; | | | |
| | | | WP1130; PLUMBAGIN; | | | |
| | | | CHIR-99021; CI-994; API-2 | | | |
| SIDS | 11 | 85 | Fluvoxamine | SCN5A; HTR2A; | 8 | −6.20 |
| Susceptibility | | | maleate; PIMOZIDE; | HTR1A; FOXM1; | | |
| Pathways | | | thiostrepton; | SLC6A4; MAOA; | | |
| | | | DEOXYGEDUNIN; AT- | NTRK2; EP300 | | |
| | | | DYRK-01; SERTRALINE | | | |
| | | | HYDROCHLORIDE; QX | | | |
| | | | 222; TCB2; FLUOXETINE; | | | |
| | | | PLUMBAGIN; DOXEPIN | | | |
| | | | HYDROCHLORIDE | | | |
| AGE/RAGE | 11 | 66 | cucurbitacin I; | PLA2G4A; RAF1; | 8 | −7.98 |
| pathway | | | Vemurafenib; FTY720; | MAPK14; AKT1; | | |
| | | | niclosamide; Vemurafenib; | IKBKB; CHUK; | | |
| | | | skepinone-L; niclosamide; SB | STAT3; MAPK1 | | |
| | | | 202190; IKK 16; CHIR-99021; | | | |
| | | | API-2 | | | |
| EGF/EGFR | 11 | 162 | cucurbitacin I; | RAF1; MAPK14; | 8 | −7.75 |
| Signaling | | | Vemurafenib; niclosamide; | PAK1; MTOR; | | |
| Pathway | | | Vemurafenib; skepinone-L; | BRAF; AKT1; | | |
| | | | SMER 3; niclosamide; SB | STAT3; MAPK1 | | |
| | | | 202190; CHIR-99021; API-2; | | | |
| | | | PF-3758309 | | | |
| IL-5 | 9 | 42 | cucurbitacin | GSK3A; RAF1; | 8 | −8.26 |
| Signaling | | | I; Vemurafenib; niclosamide; | MTOR; AKT1; | | |
| Pathway | | | Vemurafenib; SMER | GSK3B; STAT3; | | |
| | | | 3; niclosamide; LFM-A13; CHIR- | MAPK1; BTK | | |
| | | | 99021; API-2 | | | |
| TCR | 8 | 91 | Vemurafenib; Vemurafenib; | RAF1; MAPK14; | 8 | −5.88 |
| Signaling | | | skepinone-L; SB 202190; IKK | PAK1; IKBKG; | | |
| Pathway | | | 16; CHIR-99021; API-2; PF- | AKT1; IKBKB; | | |
| | | | 3758309 | CHUK; MAPK1 | | |
| Regulation | 6 | 151 | cytochalasin | RAF1; PAK1; | 8 | −9.17 |
| of Actin | | | B; Vemurafenib; Vemurafenib; | BRAF; PAK6; | | |
| Cytoskeleton | | | SCH 79797 | PAK4; ACTG1; | | |
| | | | dihydrochloride; CHIR- | MAPK1; F2R | | |
| | | | 99021; PF-3758309 | | | |
| Monoamine | 6 | 34 | 2-[(4-Phenylpiperazin-1- | HTR2A; HTR1A; | 8 | −6.32 |
| GPCRs | | | yl)methyl]-2,3- | DRD2; HRH2; | | |
| | | | dihydroimidazo[1,2- | HTR2C; ADRA1A; | | |
| | | | c]quinazolin-5(6H)- | DRD3; HRH1 | | |
| | | | one; Terciprazine; PIMOZIDE; | | | |
| | | | TCB2; FLUOXETINE; DOXEPIN | | | |
| | | | HYDROCHLORIDE | | | |
| Androgen | 14 | 89 | cucurbitacin I; MGCD- | HDAC1; KAT2B; | 7 | −8.11 |
| receptor | | | 0103; MS-275; MS-275; MS- | AKT1; PAK6; | | |
| signaling | | | 275; niclosamide; niclosamide; | GSK3B; EP300; | | |
| pathway | | | CI-994; CI- | STAT3 | | |
| | | | 994; PLUMBAGIN; CHIR- | | | |
| | | | 99021; CI-994; API-2; PF- | | | |
| | | | 3758309 | | | |
| TWEAK | 12 | 42 | MGCD-0103; MS-275; MS- | HDAC1; MAPK14; | 7 | −7.03 |
| Signaling | | | 275; MS-275; skepinone-L; CI- | AKT1; IKBKB; | | |
| Pathway | | | 994; SB 202190; CI-994; IKK | GSK3B; CHUK; | | |
| | | | 16; CHIR-99021; CI-994; API-2 | MAPK1 | | |
| TSH | 10 | 66 | cucurbitacin | RAF1; MAPK14; | 7 | −8.10 |
| signaling | | | I; Vemurafenib; niclosamide; | MTOR; BRAF; | | |
| pathway | | | Vemurafenib; skepinone- | AKT1; STAT3; | | |
| | | | L; SMER 3; niclosamide; SB | MAPK1 | | |
| | | | 202190; CHIR-99021; API-2 | | | |
| TNF alpha | 6 | 93 | Cantharidin; Vemurafenib; | RAF1; PPP2CA; | 7 | −7.28 |
| Signaling | | | Vemurafenib; IKK 16; CHIR- | IKBKG; AKT1; | | |
| Pathway | | | 99021; API-2 | IKBKB; CHUK; | | |
| | | | | MAPK1 | | |
| IL-1 | 6 | 54 | skepinone-L; SB 202190; IKK | MAPK14; IKBKG; | 7 | −5.06 |
| signaling | | | 16; CMPD-1; CHIR-99021; API-2 | AKT1; IKBKB; | | |
| pathway | | | | CHUK; MAPKAPK2; | | |
| | | | | MAPK1 | | |
| RANKL/RANK | 6 | 55 | skepinone-L; SMER 3; SB | MAPK14; IKBKG; | 7 | −5.31 |
| Signaling | | | 202190; IKK 16; CHIR- | MTOR; AKT1; | | |
| Pathway | | | 99021; API-2 | IKBKB; CHUK; | | |
| | | | | MAPK1 | | |
| Toll-like | 5 | 102 | skepinone-L; SB 202190; IKK | MAPK14; IKBKG; | 7 | −5.13 |
| receptor | | | 16; CHIR-99021; API-2 | AKT1; IKBKB; | | |
| signaling | | | | CHUK; MAPK11; | | |
| pathway | | | | MAPK1 | | |
| Integrin- | 5 | 99 | Vemurafenib; Vemurafenib; | RAF1; PAK1; | 7 | −6.05 |
| mediated | | | CHIR-99021; API-2; PF-3758309 | BRAF; AKT1; | | |
| Cell | | | | PAK6; PAK4; | | |
| Adhesion | | | | MAPK1 | | |
| Regulation | 5 | 103 | skepinone-L; SB 202190; IKK | MAPK14; IKBKG; | 7 | −5.13 |
| of toll-like | | | 16; CHIR-99021; API-2 | AKT1; IKBKB; | | |
| receptor | | | | CHUK; MAPK11; | | |
| signaling | | | | MAPK1 | | |
| pathway | | | | | | |
| Small | 3 | 18 | FTY720; “WIN 55,212-2 | PTGDR; CNR1; | 7 | −6.01 |
| Ligand | | | mesylate”; Alprostadil | CNR2; PTGIR; | | |
| GPCRs | | | | PTGER4; PTGER1; | | |
| | | | | PTGER2 | | |
| IL-6 | 13 | 44 | cucurbitacin I; MGCD- | HDAC1; AKT1; | 6 | −8.40 |
| signaling | | | 0103; MS-275; MS-275; MS- | GSK3B; EP300; | | |
| pathway | | | 275; niclosamide; niclosamide; | STAT3; MAPK1 | | |
| | | | CI-994; CI- | | | |
| | | | 994; PLUMBAGIN; CHIR- | | | |
| | | | 99021; CI-994; API-2 | | | |
| Oncostatin | 10 | 64 | cucurbitacin | RAF1; MAPK14; | 6 | −8.10 |
| M Signaling | | | I; Vemurafenib; niclosamide; | MTOR; AKT1; | | |
| Pathway | | | Vemurafenib; skepinone- | STAT3; MAPK1 | | |
| | | | L; SMER 3; niclosamide; SB | | | |
| | | | 202190; CHIR-99021; API-2 | | | |
| TSLP | 9 | 48 | cucurbitacin | MAPK14; MTOR; | 6 | −7.67 |
| Signaling | | | I; niclosamide; skepinone- | AKT1; STAT3; | | |
| Pathway | | | L; SMER 3; niclosamide; SB | MAPK1; BTK | | |
| | | | 202190; LFM-A13; CHIR- | | | |
| | | | 99021; API-2 | | | |
| Cell Cycle | 9 | 104 | MGCD-0103; MS-275; MS- | HDAC1; CDK1; | 6 | −7.54 |
| | | | 275; MS-275; CI-994; CI- | GSK3B; EP300; | | |
| | | | 994; PLUMBAGIN; CHIR- | HDAC2; HDAC3 | | |
| | | | 99021; CI-994 | | | |
| Interleukin-11 | 8 | 44 | cucurbitacin | RAF1; AKT1; | 6 | −8.49 |
| Signaling | | | I; Vemurafenib; niclosamide; | IKBKB; CHUK; | | |
| Pathway | | | Vemurafenib; niclosamide; IKK | STAT3; MAPK1 | | |
| | | | 16; CHIR-99021; API-2 | | | |
| Senescence | 6 | 106 | Vemurafenib; sb | RAF1; MAPK14; | 6 | −7.28 |
| and | | | 225002; Vemurafenib; | BRAF; CXCR2; | | |
| Autophagy | | | skepinone-L; SB 202190; | GSK3B; MAPK1 | | |
| | | | CHIR-99021 | | | |
| IL17 | 6 | 31 | cucurbitacin | IKBKG; AKT1; | 6 | −8.48 |
| signaling | | | I; niclosamide; niclosamide; IKK | IKBKB; GSK3B; | | |
| pathway | | | 16; CHIR-99021; API-2 | STAT3; MAPK1 | | |
| Prostaglandin | 2 | 31 | FTY720; Alprostadil | PTGDR; PLA2G4A; | 6 | −6.44 |
| Synthesis | | | | PTGIR; PTGER4; | | |
| and | | | | PTGER1; PTGER2 | | |
| Regulation | | | | | | |
| IL-2 | 8 | 40 | cucurbitacin | RAF1; MTOR; | 5 | −8.65 |
| Signaling | | | I; Vemurafenib; niclosamide; | AKT1; STAT3; | | |
| Pathway | | | Vemurafenib; SMER | MAPK1 | | |
| | | | 3; niclosamide; CHIR- | | | |
| | | | 99021; API-2 | | | |
| IL-3 | 8 | 49 | cucurbitacin | RAF1; PTPN6; | 5 | −8.44 |
| Signaling | | | I; Vemurafenib; niclosamide; | AKT1; STAT3; | | |
| Pathway | | | Vemurafenib; niclosamide; | MAPK1 | | |
| | | | Dephostatin; CHIR-99021; API-2 | | | |
| GPCRs, | 7 | 103 | FTY720; PIMOZIDE; “WIN | CNR1; HTR2A; | 5 | −5.54 |
| Other | | | 55,212-2 mesylate”; TCB2; | DRD3; GNRHR; | | |
| | | | FLUOXETINE; | F2R | | |
| | | | CMPD-1; SCH 79797 | | | |
| | | | dihydrochloride | | | |
| FSH | 7 | 27 | Vemurafenib; Vemurafenib; | RAF1; MAPK14; | 5 | −6.29 |
| signaling | | | skepinone-L; SMER 3; SB | MTOR; AKT1; | | |
| pathway | | | 202190; CHIR-99021; API-2 | MAPK1 | | |
| Corticotropin- | 6 | 95 | Vemurafenib; Vemurafenib; | MAPK14; BRAF; | 5 | −6.29 |
| releasing | | | skepinone-L; SB 202190; CHIR- | AKT1; GSK3B; | | |
| hormone | | | 99021; API-2 | MAPK1 | | |
| Wnt | 3 | 51 | SMER 3; CHIR-99021; API-2 | GSK3A; MTOR; | 5 | −5.04 |
| Signaling | | | | AKT1; GSK3B; | | |
| Pathway | | | | MAPK1 | | |
| Netpath | | | | | | |
| Retinoblastoma | 11 | 87 | MGCD-0103; MS-275; MS- | HDAC1; RAF1; | 4 | −7.68 |
| (RB) in | | | 275; Vemurafenib; MS- | CDK1; TOP2A | | |
| Cancer | | | 275; Vemurafenib; CI-994; CI- | | | |
| | | | 994; CHIR-99021; CI- | | | |
| | | | 994; Rubitecan | | | |
| Apoptosis- | 10 | 53 | MGCD-0103; MS-275; MS- | HDAC1; AKT1; | 4 | −7.23 |
| related | | | 275; MS-275; CI-994; CI- | MAPK1; F2R | | |
| network | | | 994; SCH 79797 | | | |
| due to | | | dihydrochloride; CHIR- | | | |
| altered | | | 99021; CI-994; API-2 | | | |
| Notch3 in | | | | | | |
| ovarian | | | | | | |
| cancer | | | | | | |
| MicroRNAs | 9 | 85 | cucurbitacin I; MS-275; MS- | RAF1; AKT1; | 4 | −10.03 |
| in | | | 275; Vemurafenib; MS- | HDAC9; STAT3 | | |
| cardiomyocyte | | | 275; niclosamide; Vemurafenib; | | | |
| hypertrophy | | | niclosamide; API-2 | | | |
| Pathogenic | 8 | 58 | cytochalasin | TUBA1A; ACTG1; | 4 | −7.58 |
| Escherichia | | | B; Parbendazole; Paclitaxel; | TUBB2C; TUBB1 | | |
| coli | | | Methiazole; Methiazole; | | | |
| infection | | | PACLITAXEL; PACLITAXEL; | | | |
| | | | Paclitaxel | | | |
| Monoamine | 8 | 32 | Fluvoxamine | MAPK14; SLC6A4; | 4 | −6.48 |
| Transport | | | maleate; skepinone-L; GBR | SLC6A2; SLC6A3 | | |
| | | | 12783; SERTRALINE | | | |
| | | | HYDROCHLORIDE; SB | | | |
| | | | 202190; GBR 13069 | | | |
| | | | dihydrochloride; FLUOXETINE; | | | |
| | | | DOXEPIN HYDROCHLORIDE | | | |
| Signaling of | 7 | 34 | cucurbitacin | RAF1; PAK1; | 4 | −8.98 |
| Hepatocyte | | | I; Vemurafenib; niclosamide; | STAT3; MAPK1 | | |
| Growth | | | Vemurafenib; niclosamide; CHIR- | | | |
| Factor | | | 99021; PF-3758309 | | | |
| Receptor | | | | | | |
| Serotonin | 7 | 19 | Vemurafenib; Terciprazine; | HTR2A; RAF1; | 4 | −6.55 |
| Receptor 2 | | | Vemurafenib; PIMOZIDE; TCB2; | HTR2C; MAPK | | |
| and ELK- | | | FLUOXETINE; CMPD-1 | APK2 | | |
| SRF/GATA4 | | | | | | |
| signaling | | | | | | |
| Interferon | 7 | 58 | cucurbitacin | MAPK14; PTPN6; | 4 | −8.51 |
| type I | | | I; niclosamide; skepinone- | MTOR; STAT3 | | |
| signaling | | | L; SMER 3; niclosamide; SB | | | |
| pathways | | | 202190; Dephostatin | | | |
| IL-7 | 5 | 25 | cucurbitacin | AKT1; GSK3B; | 4 | −9.17 |
| Signaling | | | I; niclosamide; niclosamide; | STAT3; MAPK1 | | |
| Pathway | | | CHIR-99021; API-2 | | | |
| MAPK | 5 | 29 | Vemurafenib; Vemurafenib; | RAF1; MAPK14; | 4 | −6.68 |
| Cascade | | | skepinone-L; SB 202190; CHIR- | BRAF; MAPK1 | | |
| | | | 99021 | | | |
| Alpha 6 | 5 | 31 | skepinone-L; SMER 3; SB | MAPK14; MTOR; | 4 | −5.38 |
| Beta 4 | | | 202190; CHIR-99021; API-2 | AKT1; MAPK1 | | |
| signaling | | | | | | |
| pathway | | | | | | |
| Apoptosis | 2 | 84 | IKK 16; API-2 | IKBKG; AKT1; | 4 | −4.67 |
| | | | | IKBKB; CHUK | | |
| G Protein | 1 | 92 | DIPYRIDAMOLE | PDE8B; PDE7B; | 4 | −4.41 |
| Signaling | | | | PDE8A; PDE4A | | |
| Pathways | | | | | | |
| Parkin- | 7 | 71 | Parbendazole; Paclitaxel; | TUBA1A; | 3 | −5.17 |
| Ubiquitin | | | Methiazole; Methiazole; | TUBB2C; | | |
| Proteasomal | | | PACLITAXEL; PACLITAXEL; | TUBB1 | | |
| System | | | Paclitaxel | | | |
| pathway | | | | | | |
| Cardiac | 6 | 54 | MS-275; MS- | RAF1; AKT1; | 3 | −8.88 |
| Hypertrophic | | | 275; Vemurafenib; MS- | HDAC9 | | |
| Response | | | 275; Vemurafenib; API-2 | | | |
| p38 MAPK | 4 | 34 | FTY720; skepinone-L; SB | PLA2G4A; MAPK14; | 3 | −6.13 |
| Signaling | | | 202190; CMPD-1 | MAPKAPK2 | | |
| Pathway | | | | | | |
| Extracellular | 4 | 31 | Vemurafenib; Vemurafenib; | RAF1; MTOR; | 3 | −6.93 |
| vesicle- | | | SMER 3; API-2 | AKT1 | | |
| mediated | | | | | | |
| signaling in | | | | | | |
| recipient | | | | | | |
| cells | | | | | | |
| FAS | 3 | 42 | cytochalasin B; CMPD-1; PF- | PAK1; ACTG1; | 3 | −11.15 |
| pathway | | | 3758309 | MAPKAPK2 | | |
| and Stress | | | | | | |
| induction | | | | | | |
| of HSP | | | | | | |
| regulation | | | | | | |
| Integrated | 3 | 12 | FTY720; PLUMBAGIN; API-2 | PLA2G4A; AKT1; | 3 | −5.69 |
| Pancreatic | | | | EP300 | | |
| Cancer | | | | | | |
| Pathway | | | | | | |
| Signal | 3 | 25 | FTY720; CHIR-99021; API-2 | S1PR1; AKT1; | 3 | −5.63 |
| Transduction | | | | MAPK1 | | |
| of SIP | | | | | | |
| Receptor | | | | | | |
| Glycogen | 2 | 36 | Cantharidin; CHIR-99021 | GSK3A; PPP2CA; | 3 | −8.64 |
| Metabolism | | | | GSK3B | | |
| Nicotine | 2 | 21 | PIMOZIDE; RESERPINE | DRD2; DRD3; | 3 | −5.22 |
| Activity on | | | | SLC18A2 | | |
| Dopaminergic | | | | | | |
| Neurons | | | | | | |
| T-Cell | 2 | 30 | CHIR-99021; API-2 | GSK3A; AKT1; | 3 | −4.44 |
| Receptor | | | | GSK3B | | |
| and Co- | | | | | | |
| stimulatory | | | | | | |
| Signaling | | | | | | |
| Estrogen | 1 | 27 | IKK 16 | IKBKG; IKBKB; | 3 | −4.98 |
| signaling | | | | CHUK | | |
| pathway | | | | | | |
| Serotonin | 6 | 4 | cucurbitacin | HTR2A; STAT3 | 2 | −8.86 |
| Receptor 2 | | | I; niclosamide; PIMOZIDE; | | | |
| and STAT3 | | | niclosamide; TCB2; FLUOXETINE | | | |
| Signaling | | | | | | |
| EPO | 5 | 27 | cucurbitacin | RAF1; STAT3 | 2 | −10.82 |
| Receptor | | | I; Vemurafenib; niclosamide; | | | |
| Signaling | | | Vemurafenib; niclosamide | | | |
| Serotonin | 5 | 11 | Fluvoxamine maleate; AT- | SLC6A4; MAOA | 2 | −7.01 |
| Transporter | | | DYRK-01; SERTRALINE | | | |
| Activity | | | HYDROCHLORIDE; FLUOXETINE; | | | |
| | | | DOXEPIN HYDROCHLORIDE | | | |
| TFs | 4 | 8 | cucurbitacin | AKT1; STAT3 | 2 | −10.34 |
| Regulate | | | I; niclosamide; niclosamide; API-2 | | | |
| miRNAs | | | | | | |
| related to | | | | | | |
| cardiac | | | | | | |
| hypertrophy | | | | | | |
| TGF Beta | 4 | 54 | cucurbitacin | EP300; STAT3 | 2 | −10.42 |
| Signaling | | | I; niclosamide; niclosamide; | | | |
| Pathway | | | PLUMBAGIN | | | |
| IL-9 | 4 | 17 | cucurbitacin | STAT3; MAPK1 | 2 | −10.38 |
| Signaling | | | I; niclosamide; niclosamide; | | | |
| Pathway | | | CHIR-99021 | | | |
| Serotonin | 3 | 18 | Vemurafenib; Vemurafenib; | BRAF; MAPKAPK2 | 2 | −7.28 |
| Receptor | | | CMPD-1 | | | |
| 4/6/7 and | | | | | | |
| NR3C | | | | | | |
| Signaling | | | | | | |
| Oxidative | 3 | 29 | skepinone-L; AT-DYRK-01; SB | MAPK14; MAOA | 2 | −5.76 |
| Stress | | | 202190 | | | |
| Secretion | 2 | 4 | RABEPRAZOLE | ATP4A; HRH2 | 2 | −5.47 |
| of | | | SODIUM; DOXEPIN | | | |
| Hydrochloric | | | HYDROCHLORIDE | | | |
| Acid in | | | | | | |
| Parietal | | | | | | |
| Cells | | | | | | |
| Wnt | 2 | 95 | Cantharidin; CHIR-99021 | PPP2CA; GSK3B | 2 | −8.64 |
| Signaling | | | | | | |
| Pathway | | | | | | |
| and | | | | | | |
| Pluripotency | | | | | | |
| Parkinsons | 2 | 71 | skepinone-L; SB 202190 | MAPK14; MAPK11 | 2 | −5.89 |
| Disease | | | | | | |
| Pathway | | | | | | |
| Dopamine | 2 | 13 | Cantharidin; AT-DYRK-01 | PPP2CA; MAOA | 2 | −9.13 |
| metabolism | | | | | | |
| Complement | 2 | 60 | SCH 79797 | PROC; F2R | 2 | −4.50 |
| and | | | dihydrochloride; MENADIONE | | | |
| Coagulation | | | | | | |
| Cascades | | | | | | |
| Peptide | 2 | 73 | CMPD-1; CANDESARTAN | AGTR1; GNRHR | 2 | −4.53 |
| GPCRs | | | CILEXTIL | | | |
| Hypothetical | 2 | 33 | SIB 1757; PIMOZIDE | DRD2; GRM5 | 2 | −7.11 |
| Network | | | | | | |
| for Drug | | | | | | |
| Addiction | | | | | | |
| Physiological | 3 | 25 | cucurbitacin | STAT3 | 1 | −12.33 |
| and | | | I; niclosamide; niclosamide | | | |
| Pathological | | | | | | |
| Hypertrophy | | | | | | |
| of the | | | | | | |
| Heart | | | | | | |
| TCA Cycle | 3 | 5 | cucurbitacin | STAT3 | 1 | −12.33 |
| Nutrient | | | I; niclosamide; niclosamide | | | |
| Utilization | | | | | | |
| and | | | | | | |
| Invasiveness | | | | | | |
| of | | | | | | |
| Ovarian | | | | | | |
| Cancer | | | | | | |
| Adipogenesis | 3 | 131 | cucurbitacin | STAT3 | 1 | −12.33 |
| | | | I; niclosamide; niclosamide | | | |
| Bladder | 2 | 29 | Vemurafenib; Vemurafenib | BRAF | 1 | −8.55 |
| Cancer | | | | | | |
| Sphingolipid | 1 | 19 | NVP 231 | CERK | 1 | −4.79 |
| Metabolism | | | | | | |
| Calcium | 1 | 151 | 2-[(4-Phenylpiperazin-1- | ADRA1A | 1 | −9.21 |
| Regulation | | | yl)methyl]-2,3- | | | |
| in the | | | dihydroimidazo[1,2- | | | |
| Cardiac | | | c]quinazolin-5(6H)-one | | | |
| Cell | | | | | | |
| Gastric | 1 | 31 | Rubitecan | TOP2A | 1 | −4.17 |
| cancer | | | | | | |
| network 2 | | | | | | |
| Nucleotide | 1 | 19 | Mycophenolic acid | IMPDH1 | 1 | −4.54 |
| Metabolism | | | | | | |
| Fluoropyrimidine | 1 | 33 | DIPYRIDAMOLE | SLC29A1 | 1 | −4.41 |
| Activity | | | | | | |
| Heart | 1 | 44 | CHIR-99021 | MAPK1 | 1 | −4.52 |
| Development | | | | | | |
| ACE | 1 | 17 | CANDESARTAN CILEXTIL | AGTR1 | 1 | −4.32 |
| Inhibitor | | | | | | |
| Pathway | | | | | | |
| GPCRs, | 1 | 15 | SIB 1757 | GRM5 | 1 | −7.89 |
| Class C | | | | | | |
| Metabotropic | | | | | | |
| glutamate, | | | | | | |
| pheromone | | | | | | |
| Arrhythmogenic | 1 | 74 | cytochalasin B | ACTG1 | 1 | −20.00 |
| Right | | | | | | |
| Ventricular | | | | | | |
| Cardiomyopathy | | | | | | |
| Electron | 1 | 103 | DIHYDROROTENONE | NDUFS7 | 1 | −5.14 |
| Transport | | | | | | |
| Chain | | | | | | |
| Integrated | 1 | 17 | NVP 231 | CERK | 1 | −4.79 |
| Breast | | | | | | |
| Cancer | | | | | | |
| Pathway | | | | | | |
| G1 to S cell | 1 | 69 | CHIR-99021 | CDK1 | 1 | −4.52 |
| cycle | | | | | | |
| control | | | | | | |
| Biogenic | 1 | 15 | AT-DYRK-01 | MAOA | 1 | −5.50 |
| Amine | | | | | | |
| Synthesis | | | | | | |
| ErbB | 1 | 55 | PF-3758309 | PAK4 | 1 | −4.29 |
| Signaling | | | | | | |
| Pathway | | | | | | |
| Myometrial | 1 | 156 | cytochalasin B | ACTG1 | 1 | −20.00 |
| Relaxation | | | | | | |
| and | | | | | | |
| Contraction | | | | | | |
| Pathways | | | | | | |
| Gastric | 1 | 29 | Rubitecan | TOP2A | 1 | −4.17 |
| Cancer | | | | | | |
| Network 1 | | | | | | |
| Striated | 1 | 38 | cytochalasin B | ACTG1 | 1 | −20.00 |
| Muscle | | | | | | |
| Contraction | | | | | | |
| PDGF | 1 | 12 | CHIR-99021 | MAPK1 | 1 | −4.52 |
| Pathway | | | | | | |
| DNA | 1 | 61 | CHIR-99021 | GSK3B | 1 | −4.52 |
| Damage | | | | | | |
| Response | | | | | | |
| (only ATM | | | | | | |
| dependent) | | | | | | |
| Oxidative | 1 | 60 | DIHYDROROTENONE | NDUFS7 | 1 | −5.14 |
| phosphorylation | | | | | | |
| Ovarian | 1 | 31 | Alprostadil | PTGER2 | 1 | −4.86 |
| Infertility | | | | | | |
| Genes | | | | | | |
| Wnt | 1 | 66 | CHIR-99021 | GSK3B | 1 | −4.52 |
| Signaling | | | | | | |
| Pathway | | | | | | |
| mRNA | 1 | 127 | YK 4-279 | DHX9 | 1 | −7.60 |
| Processing | | | | | | |
| Focal | 44 | 191 | Bosutinib; AT7867; SC-1; | BLK; BCL2; | 31 | 12.54 |
| Adhesion | | | Bosutinib; PF- | RAF1; TXK; | | |
| | | | 573228; SU11274(PKI- | HCK; PIK3CD; | | |
| | | | SU11274); Y-27632; VX-680; | KDR; MAPK9; | | |
| | | | 1-Naphthyl PP1; MGCD- | MAP2K2; PTK2; | | |
| | | | 265; Neratinib; TW- | ROCK1; BRAF; | | |
| | | | 37; Tozasertib; Alsterpaullone; | AKT2; MAPK8; | | |
| | | | Sunitinib | EGFR; MAP2K1; | | |
| | | | Malate; Vandetanib; Tozasertib | AKT1; PDGFRB; | | |
| | | | VX-680 (MK-0457); Erlotinib; | IGF1R; TNK2; | | |
| | | | Sunitinib malate; PCI-32765; BMS- | PTK6; GSK3B; | | |
| | | | 536924; AZD2171; RAF265; HA- | PDGFRA; SRC; | | |
| | | | 1077; SP600125; KX2- | FYN; AKT3; | | |
| | | | 391; Tivozanib; Erlotinib; | MET; ERBB2; | | |
| | | | Dasatinib; Ki8751; | MAPK1; ROCK2; | | |
| | | | PODOFILOX; AG- | FLT1 | | |
| | | | 013736; Gefitinib; HA- | | | |
| | | | 1077; Crizotinib; H 89 | | | |
| | | | dihydrochloride; HA- | | | |
| | | | 1077; ABT-737; ABT-199 (GDC- | | | |
| | | | 0199); Sorafenib; CAL- | | | |
| | | | 101; NVP-ADW742; OSI-906 | | | |
| | | | (Linsitinib); Axitinib | | | |
| GPCRs, | 11 | 261 | AZELASTINE | HTR2A; HTR1A; | 31 | 8.60 |
| Class A | | | HYDROCHLORIDE; SURAMIN; | OPRD1; MLNR; | | |
| Rhodopsin- | | | JTE 013; BNTX | CHRM2; DRD2; | | |
| like | | | maleate; Eltoprazine | SSTR4; HRH2; | | |
| | | | hydrochloride; CV | ADRA2A; HTR2C; | | |
| | | | 1808; VINCAMINE; Doxepine | ADRA1A; CHRM4; | | |
| | | | HCl; ERYTHROMYCIN | HTR1B; P2RY2; | | |
| | | | STEARATE; “7,4′- | ADORA2A; OPRK1; | | |
| | | | DIHYDROXYFLAVONE”; “L- | P2RY11; P2RY1; | | |
| | | | 803,087 trifluoroacetate” | CHRM1; ADRA1B; | | |
| | | | | HRH3; HTR2B; | | |
| | | | | ADRA2C; ADRA2B; | | |
| | | | | CHRM5; P2RY13; | | |
| | | | | HRH1; CHRM3; | | |
| | | | | P2RY10; ADRA1D; | | |
| | | | | OPRM1 | | |
| EGF/EGFR | 35 | 162 | Bosutinib; AT7867; SC- | RAF1; AURKA; | 26 | 13.80 |
| Signaling | | | 1; Bosutinib; PF-573228; KW | ABL1; MAPK9; | | |
| Pathway | | | 2449; Y-27632; VX-680; 1- | PRKCA; MAPK3; | | |
| | | | Naphthyl PP1; Neratinib; Cyt387; Go | PRKCD; MAP2K2; | | |
| | | | 6976; Tozasertib; TG- | PTK2; ROCK1; | | |
| | | | 101348; INCB018424; Vandetanib; | MAP3K2; RPS6KA3; | | |
| | | | Tozasertib VX-680 (MK- | BRAF; MAPK8; | | |
| | | | 0457); H-7 dihydrochloride; | EGFR; MAP2K1; | | |
| | | | Erlotinib; sotrastaurin; | AKT1; PRKCB; | | |
| | | | C-1; RAF265; HA-1077; SP600125; | JAK2; TNK2; | | |
| | | | KX2-391; INCB018424; Erlotinib; | PTK6; JAK1; | | |
| | | | Dasatinib; | SRC; RPS6KA5; | | |
| | | | AZD1480; Gefitinib; HA- | ERBB2; MAPK1 | | |
| | | | 1077; H 89 | | | |
| | | | dihydrochloride; HA- | | | |
| | | | 1077; Sorafenib; MLN8237 | | | |
| MAPK | 28 | 168 | Bosutinib; AT7867; SC- | PPP3CA; TGFBR2; | 26 | 12.36 |
| Signaling | | | 1; Bosutinib; Neratinib; Sunitinib | RAF1; MAPK9; | | |
| Pathway | | | Malate; Vandetanib; H-7 | MAPK3; PRKCD; | | |
| | | | dihydrochloride; Erlotinib; | PRKACA; MAP2K2; | | |
| | | | sotrastaurin; Sunitinib malate; C- | MKNK1; MAP3K2; | | |
| | | | 1; AZD2171; RAF265; SP60012 | RPS6KA3; BRAF; | | |
| | | | 5; VER155008; Erlotinib; CGP | AKT2; MAPK8; | | |
| | | | 57380; “Dibutyryl-cAMP, | EGFR; MAP2K1; | | |
| | | | sodium salt”; Gefitinib; HA- | AKT1; PRKCH; | | |
| | | | 1077; H 89 dihydrochloride; | PDGFRB; TGFBR1; | | |
| | | | HA-1077; Phorbol 12-Myristate | MAPK10; FGFR1; | | |
| | | | 13-Acetate; Sorafenib; APIGENIN; | RPS6KA5; AKT3; | | |
| | | | cyclosporine; LY 364947 | MAPK1; HSPA1A | | |
| Insulin | 21 | 160 | Bosutinib; AT7867; SC- | INSR; PRKAA2; | 26 | 13.46 |
| Signaling | | | 1; Bosutinib; Go | RAF1; PIK3CD; | | |
| | | | 6976; Alsterpaullone; | MAPK9; PRKCA; | | |
| | | | Dorsomorphin dihydrochloride; H-7 | MAPK3; PRKCD; | | |
| | | | dihydrochloride; sotrastaurin; | MAP2K2; MAP3K2; | | |
| | | | B MS-5369 24; C- | RPS6KA3; AKT2; | | |
| | | | 1; SP600125; OLEANOIC | MAPK8; MAP2K1; | | |
| | | | ACID; triptolide; PODOFILOX; | PTPN1; PRKCQ; | | |
| | | | H 89 dihydrochloride; Sorafenib; | AKT1; PRKCH; | | |
| | | | APIGENIN; CAL-101; NVP- | MAP4K5; IGF1R; | | |
| | | | ADW742; OSI-906 (Linsitinib) | GSK3B; MAPK10; | | |
| | | | | PRKAA1; XBP1; | | |
| | | | | RPS6KA5; MAPK1 | | |
| Leptin | 37 | 78 | Bosutinib; AT7867; SC- | PRKAA2; BCL2L1; | 22 | 13.15 |
| signaling | | | 1; Bosutinib; PF-573228; Y- | RAF1; MAPK3; | | |
| pathway | | | 27632; VX-680; 1-Naphthyl | MAP2K2; PTK2; | | |
| | | | PP1; Neratinib; TW- | ROCK1; MAPK8; | | |
| | | | 37; Cyt387; Tozasertib; | EGFR; MAP2K1; | | |
| | | | Alsterpaullone; TG- | PTPN1; AKT1; | | |
| | | | 101348; INCB018424; Vandetanib; | IGF1R; JAK2; | | |
| | | | Dorsomorphin | GSK3B; JAK1; | | |
| | | | dihydrochloride; Tozasertib | SRC; PRKAA1; | | |
| | | | VX-680 (MK- | FYN; ERBB2; | | |
| | | | 0457); Erlotinib; BMS- | ROCK2; MAPK1 | | |
| | | | 536924; HA- | | | |
| | | | 1077; SP600125; KX2- | | | |
| | | | 391; OLEANOIC | | | |
| | | | ACID; INCB018424; Erlotinib; | | | |
| | | | Dasatinib; PODOFILOX; AZD1480; | | | |
| | | | Gefitinib; HA-1077; H 89 | | | |
| | | | dihydrochloride; HA- | | | |
| | | | 1077; ABT- | | | |
| | | | 737; Sorafenib; NVP- | | | |
| | | | ADW742; OSI-906 (Linsitinib) | | | |
| BDNF | 27 | 146 | Bosutinib; AT7867; SC- | CDK5; PRKAA2; | 22 | 13.68 |
| signaling | | | 1; Bosutinib; VX-680; 1- | RAF1; GRIA2; | | |
| pathway | | | Naphthyl | MAPK9; MAPK3; | | |
| | | | PP1; Cyt387; Tozasertib; | PRKCD; MAP2K2; | | |
| | | | Alsterpaullone; TG- | CSNK2A1; MAP3K2; | | |
| | | | 101348; INCB018424; Vandetanib; | RPS6KA3; MAPK8; | | |
| | | | Dorsomorphin | MAP2K1; AKT1; | | |
| | | | dihydrochloride; Tozasertib | JAK2; GSK3B; | | |
| | | | VX-680 (MK- | MAPK10; SRC; | | |
| | | | 0457); sotrastaurin; C- | PRKAA1; FYN; | | |
| | | | 1; SP600125; KX2- | RPS6KA5; MAPK1 | | |
| | | | 391; INCB018424; Dasatinib; | | | |
| | | | BARBITAL; N9- | | | |
| | | | isopropylolomoucine; | | | |
| | | | AZD1480; H 89 | | | |
| | | | dihydrochloride; SCH727965; | | | |
| | | | Sorafenib; APIGENIN | | | |
| Oncostatin | 29 | 64 | Bosutinib; AT7867; SC- | RAF1; PRKCE; | 20 | 14.00 |
| M Signaling | | | 1; Bosutinib; Y-27632; VX- | MAPK9; PRKCA; | | |
| Pathway | | | 680; 1-Naphthyl | MAPK3; PRKCD; | | |
| | | | PP1; Arcyriaflavin | MAP2K2; MAPK8; | | |
| | | | A; Cyt387; Go | MAP2K1; AKT1; | | |
| | | | 6976; Tozasertib; TG- | PRKCH; PRKCB; | | |
| | | | 101348; INCB018424; Vandetanib; | JAK2; JAK3; TYK2; | | |
| | | | Tozasertib VX-680 (MK- | JAK1; SRC; | | |
| | | | 0457); H-7 | SERPINE1; CDK2; | | |
| | | | dihydrochloride; sotrastaurin; | MAPK1 | | |
| | | | C-1; CDK9 inhibitor | | | |
| | | | 14; SP600125; KX2- | | | |
| | | | 391; INCB018424; Dasatinib; | | | |
| | | | N9-isopropylolomoucine; AZD1480; | | | |
| | | | (−)-Epigallocatechin | | | |
| | | | Gallate; H 89 | | | |
| | | | dihydrochloride; SCH727965; | | | |
| | | | Sorafenib | | | |
| Regulation | 23 | 151 | Bosutinib; SC-1; Bosutinib; PF- | CHRM2; RAF1; | 19 | 12.92 |
| of Actin | | | 573228; Y- | PIK3CD; MAPK3; | | |
| Cytoskeleton | | | 27632; Neratinib; Sunitinib | MAP2K2; PTK2; | | |
| | | | Malate; Vandetanib; Erlotinib; | ROCK1; CHRM4; | | |
| | | | Sunitinib | BRAF; EGFR; | | |
| | | | malate; AZD2171; RAF265; HA- | MAP2K1; PDGFRB; | | |
| | | | 1077; Erlotinib; Ki8751; Gefitinib; | CHRM1; PDGFRA; | | |
| | | | HA-1077; H 89 | CHRM5; FGFR1; | | |
| | | | dihydrochloride; HA- | CHRM3; ROCK2; | | |
| | | | 1077; VINCAMINE; Doxepine | MAPK1 | | |
| | | | HCl; Sorafenib; CAL-101 | | | |
| Monoamine | 4 | 34 | AZELASTINE | HTR2A; HTR1A; | 19 | 9.17 |
| GPCRs | | | HYDROCHLORIDE; Eltoprazine | CHRM2; DRD2; | | |
| | | | hydrochloride; VINCAMINE; | HRH2; ADRA2A; | | |
| | | | Doxepine HCl | HTR2C; ADRA1A; | | |
| | | | | CHRM4; HTR1B; | | |
| | | | | CHRM1; ADRA1B; | | |
| | | | | HTR2B; ADRA2C; | | |
| | | | | ADRA2B; CHRM5; | | |
| | | | | HRH1; CHRM3; | | |
| | | | | ADRA1D | | |
| Cell Cycle | 18 | 104 | Bosutinib; Bosutinib; KW | HDAC1; HDAC6; | 18 | 12.54 |
| | | | 2449; 1-Naphthyl | HDAC4; PLK1; | | |
| | | | PP1; Arcyriaflavin | CDK4; CDK1; | | |
| | | | A; apicidin; Alsterpaullone; CD | ABL1; CDK6; | | |
| | | | K9 inhibitor 14; Dasatinib; N9- | HDAC5; EP300; | | |
| | | | isopropylolomoucine; MK- | WEE1; GSK3B; | | |
| | | | 1775; KU-55933; rigosertib; (−)- | HDAC8; ATM; | | |
| | | | Epigallocatechin | HDAC2; CDK2; | | |
| | | | Gallate; SCH727965; Belinostat; | HDAC7; HDAC3 | | |
| | | | APIGENIN; Pandacostat | | | |
| Calcium | 15 | 151 | Bosutinib; Bosutinib; Y- | ADCY5; ADCY2; | 18 | 12.27 |
| Regulation | | | 27632; Go 6976; H-7 | CHRM2; PRKCE; | | |
| in the | | | dihydrochloride; sotrastaurin; | PRKCA; ADRA1A; | | |
| Cardiac | | | C-1; “Dibutyryl-cAMP, sodium | PRKCD; PRKACA; | | |
| Cell | | | salt”; HA-1077; H 89 | CHRM4; PRKCQ; | | |
| | | | dihydrochloride; HA- | PRKCH; CHRM1; | | |
| | | | 1077; Phorbol 12-Myristate | ADRA1B; PRKD1; | | |
| | | | 13-Acetate; VINCAMINE; Doxepine | CHRM5; CAMK2G; | | |
| | | | HCl; COLFORSIN | CHRM3; ADRA1D | | |
| Kit | 33 | 59 | Bosutinib; AT7867; SC- | BCL2; KIT; RAF1; | 17 | 13.04 |
| receptor | | | 1; Bosutinib; VX-680; 1- | LYN; PRKCA; | | |
| signaling | | | Naphthyl PP1; TW- | MAPK3; MAP2K2; | | |
| pathway | | | 37; Cyt387; Go | RPS6KA3; MAPK8; | | |
| | | | 6976; Tozasertib; Sunitinib | MAP2K1; AKT1; | | |
| | | | Malate; TG- | JAK2; EP300; | | |
| | | | 101348; INCB018424; Vandetanib; | SRC; FYN; | | |
| | | | Tozasertib VX-680 (MK- | MAPK1; BTK | | |
| | | | 0457); H-7 | | | |
| | | | dihydrochloride; sotrastaurin; | | | |
| | | | Sunitinib malate; PCI- | | | |
| | | | 32765; C-1; | | | |
| | | | AZD2171; SP600125; KX2- | | | |
| | | | 391; INCB018424; Dasatinib; | | | |
| | | | AG-013736; AZD1480; (−)- | | | |
| | | | Epigallocatechin Gallate; H 89 | | | |
| | | | dihydrochloride; ABT- | | | |
| | | | 737; ABT-199 (GDC-0199); | | | |
| | | | Sorafenib; NVP-ADW742 | | | |
| IL-3 | 30 | 49 | Bosutinib; AT7867; SC- | SYK; BCL2; | 17 | 12.90 |
| Signaling | | | 1; Bosutinib; VX-680; 1- | BCL2L1; RAF1; | | |
| Pathway | | | Naphthyl PP1; TW- | LYN; HCK; | | |
| | | | 37; Cyt387; Tozasertib; TG- | PIK3CD; MAPK3; | | |
| | | | 101348; INCB018424; Vandetanib; | PRKACA; MAPK8; | | |
| | | | Tozasertib VX-680 (MK- | MAP2K1; AKT1; | | |
| | | | 0457); H-7 dihydrochloride; C-1; | JAK2; JAK1; | | |
| | | | SP600125; KX2- | SRC; FYN; | | |
| | | | 391; INCB018424; Dasatinib; | MAPK1 | | |
| | | | ER 27319 maleate; “Dibutyryl- | | | |
| | | | cAMP, sodium | | | |
| | | | salt”; AZD1480; HA-1077; H 89 | | | |
| | | | dihydrochloride; HA- | | | |
| | | | 1077; Phorbol 12-Myristate | | | |
| | | | 13-Acetate; ABT-737; ABT-199 | | | |
| | | | (GDC-0199); Sorafenib; CAL-101 | | | |
| TGF beta | 26 | 135 | Bosutinib; AT7867; SC- | HDAC1; TGFBR2; | 17 | 14.40 |
| Signaling | | | 1; Bosutinib; PF- | RAF1; MAPK9; | | |
| Pathway | | | 573228; SU11274(PKI- | MAPK3; MAP2K2; | | |
| | | | SU11274); Y-27632; VX-680; 1- | PTK2; ROCK1; | | |
| | | | Naphthyl PP1; apicidin; Tozasertib; | MAPK8; MAP2K1; | | |
| | | | Vandetanib; Tozasertib VX-680 | AKT1; TGFBR1; | | |
| | | | (MK-0457); HA- | EP300; SRC; | | |
| | | | 1077; SP600125; KX2- | MET; SIK1; MAPK1 | | |
| | | | 391; Dasatinib; (−)- | | | |
| | | | Epigallocatechin Gallate; HA- | | | |
| | | | 1077; Crizotinib; H 89 | | | |
| | | | dihydrochloride; HA- | | | |
| | | | 1077; Belinostat; Sorafenib; LY | | | |
| | | | 364947; Pandacostat | | | |
| B Cell | 17 | 100 | Bosutinib; AT7867; SC- | SYK; LCK; BLK; | 17 | 16.07 |
| Receptor | | | 1; Bosutinib; VX-680; 1- | RAF1; LYN; | | |
| Signaling | | | Naphthyl PP1; Tozasertib; | MAPK9; MAPK3; | | |
| Pathway | | | Alsterpaullone; | PRKCD; MAP2K2; | | |
| | | | Tozasertib VX-680 (MK- | BRAF; MAPK8; | | |
| | | | 0457); sotrastaurin; PCI- | MAP2K1; AKT1; | | |
| | | | 32765; C-1; RAF265; | GSK3B; FYN; | | |
| | | | SP600125; ER 27319 maleate; | MAPK1; BTK | | |
| | | | H 89 dihydrochloride; Sorafenib | | | |
| AGE/RAGE | 34 | 66 | Bosutinib; AT7867; SC- | INSR; RAF1; | 16 | 13.16 |
| pathway | | | 1; Bosutinib; Y-27632; VX- | MAPK9; PRKCA; | | |
| | | | 680; 1-Naphthyl | MAPK3; PRKCD; | | |
| | | | PP1; Neratinib; Cyt387; Go | ROCK1; MAPK8; | | |
| | | | 6976; Tozasertib; TG- | EGFR; MAP2K1; | | |
| | | | 101348; INCB018424; Vandetanib; | AKT1; PRKCB; | | |
| | | | Tozasertib VX-680 (MK- | JAK2; MMP7; | | |
| | | | 0457); H-7 | SRC; MAPK1 | | |
| | | | dihydrochloride; Erlotinib; | | | |
| | | | sotrastaurin; BMS-536924; C- | | | |
| | | | 1; HA-1077; SP600125; KX2- | | | |
| | | | 391; INCB018424; Erlotinib; | | | |
| | | | Dasatinib; AZD1480; Gefitinib; (−)- | | | |
| | | | Epigallocatechin Gallate; HA- | | | |
| | | | 1077; H 89 | | | |
| | | | dihydrochloride; HA- | | | |
| | | | 1077; Sorafenib; OSI-906 | | | |
| | | | (Linsitinib) | | | |
| Corticotropin- | 19 | 95 | Bosutinib; AT7867; SC- | BCL2; PRKAA2; | 16 | 15.28 |
| releasing | | | 1; Bosutinib; PF-573228; TW- | MAPK9; PRKCA; | | |
| hormone | | | 37; Go | MAPK3; PRKCD; | | |
| | | | 6976; Alsterpaullone; | PTK2; BRAF; | | |
| | | | Dorsomorphin dihydrochloride; | MAPK8; MAP2K1; | | |
| | | | H-7 dihydrochloride; sotrastaurin; | PRKCQ; AKT1; | | |
| | | | C-1; RAF265; SP600125; | PRKCB; GSK3B; | | |
| | | | ETHOSUXIMIDE; H 89 | CACNA1H; MAPK1 | | |
| | | | dihydrochloride; ABT- | | | |
| | | | 737; ABT-199 (GDC-0199); | | | |
| | | | Sorafenib | | | |
| TSLP | 19 | 48 | Bosutinib; AT7867; SC- | LCK; LYN; HCK; | 15 | 15.70 |
| Signaling | | | 1; Bosutinib; VX-680; 1- | MAPK9; MAPK3; | | |
| Pathway | | | Naphthyl | MAP2K2; MAPK8; | | |
| | | | PP1; Cyt387; Tozasertib; TG- | MAP2K1; AKT1; | | |
| | | | 101348; INCB018424; Vandetanib; | JAK2; JAK1; | | |
| | | | Tozasertib VX-680 (MK- | SRC; FYN; | | |
| | | | 0457); PCI- | MAPK1; BTK | | |
| | | | 32765; SP600125; KX2- | | | |
| | | | 391; INCB018424; Dasatinib; | | | |
| | | | AZD1480; H 89 dihydrochloride | | | |
| Integrin- | 21 | 99 | Bosutinib; AT7867; SC- | RAF1; MAP2K2; | 14 | 15.44 |
| mediated | | | 1; Bosutinib; PF-573228; Y- | PTK2; ROCK1; | | |
| Cell | | | 27632; VX-680; 1-Naphthyl | BRAF; AKT2; | | |
| Adhesion | | | PP1; Tozasertib; Vandetanib; | MAP2K1; AKT1; | | |
| | | | Tozasertib VX-680 (MK- | MAPK10; SRC; | | |
| | | | 0457); RAF265; HA- | FYN; AKT3; | | |
| | | | 1077; SP600125; KX2- | ROCK2; MAPK1 | | |
| | | | 391; Dasatinib; HA-1077; H 89 | | | |
| | | | dihydrochloride; HA- | | | |
| | | | 1077; Sorafenib; APIGENIN | | | |
| IL-6 | 19 | 44 | Bosutinib; AT7867; SC- | HDAC1; BCL2L1; | 14 | 14.59 |
| signaling | | | 1; Bosutinib; TW- | HCK; MAPK3; | | |
| pathway | | | 37; apicidin; Cyt387; Alsterpaullone; | PRKCD; MAP2K2; | | |
| | | | TG-101348; INCB018424; | MAP2K1; AKT1; | | |
| | | | sotrastaurin; C-1; | JAK2; EP300; | | |
| | | | INCB018424; AZD1480; (−)- | TYK2; GSK3B; | | |
| | | | Epigallocatechin Gallate; H 89 | JAK1; MAPK1 | | |
| | | | dihydrochloride; ABT- | | | |
| | | | 737; Belinostat; Pandacostat | | | |
| TSH | 25 | 66 | Bosutinib; AT7867; SC- | PDE4D; ADCY2; | 13 | 13.92 |
| signaling | | | 1; Bosutinib; VX-680; 1- | RAF1; CDK4; | | |
| pathway | | | Naphthyl PP1; Arcyriaflavin | MAPK3; BRAF; | | |
| | | | A; Cyt387; Tozasertib; TG- | MAP2K1; AKT1; | | |
| | | | 101348; INCB018424; Vandetanib; | JAK2; JAK1; | | |
| | | | Tozasertib VX-680 (MK- | SRC; CDK2; | | |
| | | | 0457); RAF265; CDK9 inhibitor | MAPK1 | | |
| | | | 14; KX2-391; INCB018424; Dasatinib; | | | |
| | | | N9-isopropylolomoucine; AZD1480; | | | |
| | | | H 89 dihydrochloride; SCH727965; | | | |
| | | | Zardaverine; Sorafenib; | | | |
| | | | COLFORSIN | | | |
| IL-5 | 19 | 42 | Bosutinib; AT7867; SC- | SYK; BCL2; RAF1; | 13 | 14.53 |
| Signaling | | | 1; Bosutinib; Ro 31-8220 | LYN; MAPK3; | | |
| Pathway | | | mesylate; TW- | MAP2K2; PIM1; | | |
| | | | 37; Cyt387; Alsterpaullone; TG- | MAP2K1; AKT1; | | |
| | | | 101348; INCB018424; PCI- | JAK2; GSK3B; | | |
| | | | 32765; INCB018424; ER 27319 | MAPK1; BTK | | |
| | | | maleate; AZD1480; H 89 | | | |
| | | | dihydrochloride; ABT- | | | |
| | | | 737; ABT-199 (GDC-0199); | | | |
| | | | Sorafenib; APIGENIN | | | |
| TCR | 15 | 91 | Bosutinib; AT7867; SC- | LCK; RAF1; | 13 | 16.54 |
| Signaling | | | 1; Bosutinib; VX-680; 1- | MAPK9; MAPK3; | | |
| Pathway | | | Naphthyl PP1; Tozasertib; | PRKCD; MAP2K2; | | |
| | | | Tozasertib VX-680 (MK- | MAPK8; MAP2K1; | | |
| | | | 0457); sotrastaurin; C- | PRKCQ; AKT1; FYN; | | |
| | | | 1; SP600125; BNTX maleate; | MAPK1; OPRM1 | | |
| | | | H 89 dihydrochloride; Sorafenib; | | | |
| | | | “7,4′-DIHYDROXYFLAVONE” | | | |
| Androgen | 27 | 89 | Bosutinib; AT7867; Bosutinib; | HDAC1; KAT2B; | 12 | 13.58 |
| receptor | | | PF-573228; Y-27632; VX-680; 1- | PTK2; ROCK1; | | |
| signaling | | | Naphthyl PP1; Neratinib; apicidin; | EGFR; AKT1; | | |
| pathway | | | Tozasertib; Alsterpaullone; Vandetanib; | SIRT1; EP300; | | |
| | | | Salermide; Tozasertib VX- | GSK3B; SRC; | | |
| | | | 680 (M K-0457); Erlotinib; HA- | ROCK2; NR2C2 | | |
| | | | 1077; KX2-391; Erlotinib; | | | |
| | | | Dasatinib; Gefitinib; | | | |
| | | | (−)-Epigallocatechin | | | |
| | | | Gallate; HA-1077; H 89 | | | |
| | | | dihydrochloride; HA- | | | |
| | | | 1077; Belinostat; Retinoic | | | |
| | | | acid; Pandacostat | | | |
| Senescence | 26 | 106 | Bosutinib; SC-1; Bosutinib; VX- | BCL2; RAF1; | 12 | 12.45 |
| and | | | 680; 1-Naphthyl | CDK4; BRAF; | | |
| Autophagy | | | PP1; Arcyriaflavin A; TW- | MAP2K1; CDK6; | | |
| | | | 37; Tozasertib; Alsterpaullone; | IGF1R; GSK3B; | | |
| | | | Vandetanib; Tozasertib VX- | SRC; SERPINE1; | | |
| | | | 680 (MK-0457); BMS- | CDK2; MAPK1 | | |
| | | | 536924; RAF265; CDK9 | | | |
| | | | inhibitor 14; KX2- | | | |
| | | | 391; Dasatinib; PODOFILOX; N9- | | | |
| | | | isopropylolomoucine; (−)- | | | |
| | | | Epigallocatechin Gallate; ABT- | | | |
| | | | 737; SCH727965; ABT-199 | | | |
| | | | (GDC-0199); Sorafenib; | | | |
| | | | APIGENIN; NVP- | | | |
| | | | ADW742; OSI-906 (Linsitinib) | | | |
| Interleukin- | 21 | 44 | Bosutinib; AT7867; SC- | BCL2; RAF1; | 12 | 14.86 |
| 11 | | | 1; Bosutinib; VX-680; 1- | MAPK3; MAP2K2; | | |
| Signaling | | | Naphthyl PP1; TW- | MAP2K1; AKT1; | | |
| Pathway | | | 37; Cyt387; Tozasertib; TG- | JAK2; TYK2; | | |
| | | | 101348; INCB018424; Vandetanib; | JAK1; SRC; | | |
| | | | Tozasertib VX-680 (MK- | FYN; MAPK1 | | |
| | | | 0457); KX2- | | | |
| | | | 391; INCB018424; Dasatinib; | | | |
| | | | AZD1480; H 89 | | | |
| | | | dihydrochloride; ABT- | | | |
| | | | 737; ABT-199 (GDC-0199); | | | |
| | | | Sorafenib | | | |
| IL-2 | 19 | 40 | Bosutinib; AT7867; SC- | SYK; LCK; BCL2; | 12 | 15.29 |
| Signaling | | | 1; Bosutinib; VX-680; 1- | RAF1; MAPK3; | | |
| Pathway | | | Naphthyl PP1; TW- | MAP2K2; MAP2K1; | | |
| | | | 37; Cyt387; Tozasertib; TG- | AKT1; JAK3; JAK1; | | |
| | | | 101348; INCB018424; Tozasertib | FYN; MAPK1 | | |
| | | | VX-680 (MK- | | | |
| | | | 0457); INCB018424; ER 27319 | | | |
| | | | maleate; AZD1480; H 89 | | | |
| | | | dihydrochloride; ABT- | | | |
| | | | 737; ABT-199 (GDC-0199); | | | |
| | | | Sorafenib | | | |
| G Protein | 14 | 92 | Y-27632; Triazolothiadiazine; Go | ADCY5; PPP3CA; | 12 | 9.68 |
| Signaling | | | 6976; H-7 | PDE4D; ADCY2; | | |
| Pathways | | | dihydrochloride; sotrastaurin; | PRKCE; PRKCA; | | |
| | | | C-1; “Dibutyryl-cAMP, sodium | PRKCD; PRKACA; | | |
| | | | salt”; HA-1077; H 89 | PRKCQ; PRKCH; | | |
| | | | dihydrochloride; HA- | PRKD1; PDE4A | | |
| | | | 1077; Phorbol 12-Myristate | | | |
| | | | 13-Acetate; Zardaverine; | | | |
| | | | cyclosporine; COLFORSIN | | | |
| MicroRNAs | 12 | 85 | AT7867; Y-27632; CDK9 | RAF1; HDAC4; | 12 | 10.76 |
| in | | | inhibitor 14; HA-1077; HA- | PIK3CD; ROCK1; | | |
| cardiomyocyte | | | 1077; H 89 | CDK9; AKT2; | | |
| hypertrophy | | | dihydrochloride; HA- | HDAC9; AKT1; | | |
| | | | 1077; SCH727965; Belinostat; | HDAC5; CDK7; | | |
| | | | Sorafenib; CAL- | HDAC7; ROCK2 | | |
| | | | 101; Pandacostat | | | |
| Retinoblastoma | 22 | 87 | Bosutinib; Bosutinib; KW | HDAC1; RAF1; | 11 | 12.00 |
| (RB) in | | | 2449; 1-Naphthyl | CDK1; CDK4; | | |
| Cancer | | | PP1; Arcyriaflavin | PLK4; ABL1; | | |
| | | | A; apicidin; Daunorubicin; | TOP2A; CDK6; | | |
| | | | Alsterpaullone; AZD7762; CDK9 | WEE1; CHEK1; | | |
| | | | inhibitor | CDK2 | | |
| | | | 14; Dasatinib; PODOFILOX; N9- | | | |
| | | | isopropylolomoucine; | | | |
| | | | Bisantrene dihydrochloride; MK- | | | |
| | | | 1775; SCH727965; Belinostat; | | | |
| | | | Doxorubicin; Sorafenib; | | | |
| | | | APIGENIN; Pandacostat; Axitinib | | | |
| SIDS | 18 | 85 | Sunitinib Malate; TG- | HTR2A; HTR1A; | 11 | 8.59 |
| Susceptibility | | | 101348; Vandetanib; H-7 | CHRM2; GABRA1; | | |
| Pathways | | | dihydrochloride; Sunitinib | SLC6A4; PRKACA; | | |
| | | | malate; C- | CHRNA4; KCNH2; | | |
| | | | 1; BARBITAL; “Dibutyryl-cAMP, | CHRNB2; EP300; | | |
| | | | sodium salt”; Eltoprazine | RET | | |
| | | | hydrochloride; (−)- | | | |
| | | | Epigallocatechin Gallate; HA- | | | |
| | | | 1077; H 89 | | | |
| | | | dihydrochloride; HA- | | | |
| | | | 1077; Phorbol 12-Myristate | | | |
| | | | 13-Acetate; UB 165 | | | |
| | | | fumarate; Doxepine | | | |
| | | | HCl; ERYTHROMYCIN | | | |
| | | | STEARATE; Sorafenib | | | |
| RANKL/RANK | 15 | 55 | Bosutinib; AT7867; SC- | SYK; LYN; MAPK9; | 11 | 17.73 |
| Signaling | | | 1; Bosutinib; PF-573228; VX- | MAPK3; PTK2; | | |
| Pathway | | | 680; 1-Naphthyl | AKT2; MAPK8; | | |
| | | | PP1; Tozasertib; Vandetanib; | MAP2K1; AKT1; | | |
| | | | Tozasertib VX-680 (MK- | SRC; MAPK1 | | |
| | | | 0457); SP600125; KX2- | | | |
| | | | 391; Dasatinib; ER 27319 | | | |
| | | | maleate; H 89 | | | |
| | | | dihydrochloride | | | |
| Myometrial | 14 | 156 | Bosutinib; Bosutinib; Y- | ADCY5; PDE4D; | 11 | 12.63 |
| Relaxation | | | 27632; Go 6976; H-7 | ADCY2; PRKCE; | | |
| and | | | dihydrochloride; sotrastaurin; | PRKCA; PRKCD; | | |
| Contraction | | | C-1; “Dibutyryl-cAMP, sodium | PRKACA; PRKCQ; | | |
| Pathways | | | salt”; HA-1077; H 89 | PRKCH; PRKD1; | | |
| | | | dihydrochloride; HA- | CAMK2G | | |
| | | | 1077; Phorbol 12-Myristate | | | |
| | | | 13-Acetate; Zardaverine; | | | |
| | | | COLFORSIN | | | |
| Wnt | 10 | 95 | Y-27632; Go | PPARD; PRKCE; | 11 | 10.68 |
| Signaling | | | 6976; Alsterpaullone; H-7 | MAPK9; PRKCA; | | |
| Pathway | | | dihydrochloride; sotrastaurin; | PRKCD; PRKCQ; | | |
| and | | | C-1; SP600125; (−)- | PRKCH; MMP7; | | |
| Pluripotency | | | Epigallocatechin | GSK3B; PRKD1; | | |
| | | | Gallate; Retinoic | MAPK10 | | |
| | | | acid; APIGENIN | | | |
| Toll-like | 8 | 102 | Bosutinib; AT7867; SC- | PIK3CD; MAPK9; | 11 | 19.88 |
| receptor | | | 1; Bosutinib; SP600125; H 89 | MAPK3; MAP2K2; | | |
| signaling | | | dihydrochloride; APIGENIN; | AKT2; MAPK8; | | |
| pathway | | | CAL-101 | MAP2K1; AKT1; | | |
| | | | | MAPK10; AKT3; | | |
| | | | | MAPK1 | | |
| Regulation | 8 | 103 | Bosutinib; AT7867; SC- | PIK3CD; MAPK9; | 11 | 19.88 |
| of toll-like | | | 1; Bosutinib; SP600125; H 89 | MAPK3; MAP2K2; | | |
| receptor | | | dihydrochloride; APIGENIN; | AKT2; MAPK8; | | |
| signaling | | | CAL-101 | MAP2K1; AKT1; | | |
| pathway | | | | MAPK10; AKT3; | | |
| | | | | MAPK1 | | |
| Pathogenic | 18 | 58 | Bosutinib; Bosutinib; KW | ABL1; PRKCA; | 10 | 13.21 |
| Escherichia | | | 2449; Y-27632; 1-Naphthyl | TUBA1A; ROCK1; | | |
| coli | | | PP1; Go 6976; H-7 | TUBA4A; TUBB; | | |
| infection | | | dihydrochloride; sotrastaurin; | TUBA1B; FYN; | | |
| | | | C-1; HA- | TUBB1; ROCK2 | | |
| | | | 1077; Dasatinib; Colchicine; | | | |
| | | | Vinbiastine | | | |
| | | | sulfate; PODOFILOX; | | | |
| | | | COLCHICINE; HA-1077; H 89 | | | |
| | | | dihydrochloride; HA-1077 | | | |
| IL-7 | 14 | 25 | Bosutinib; AT7867; SC- | BCL2L1; MAPK3; | 10 | 17.09 |
| Signaling | | | 1; Bosutinib; 1-Naphthyl | MAP2K2; MAP2K1; | | |
| Pathway | | | PP1; TW- | AKT1; JAK3; | | |
| | | | 37; Cyt387; Alsterpaullone; TG- | GSK3B; JAK1; | | |
| | | | 101348; INCB018424; INCB018424; | FYN; MAPK1 | | |
| | | | AZD1480; H 89 | | | |
| | | | dihydrochloride; ABT-737 | | | |
| IL-4 | 12 | 56 | AT7867; SC-1; AZELASTINE | PIK3CD; MAPK3; | 10 | 13.46 |
| Signaling | | | HYDROCHLORIDE; Cyt387; TG- | AKT1; JAK2; | | |
| Pathway | | | 101348; INCB018424; INCB018424; | JAK3; EP300; | | |
| | | | AZD1480; (−)- | TYK2; JAK1; | | |
| | | | Epigallocatechin Gallate; H 89 | HRH1; MAPK1 | | |
| | | | dihydrochloride; Doxepine | | | |
| | | | HCl; CAL-101 | | | |
| Wnt | 11 | 51 | AT7867; SC-1; Go | TEK; MAPK9; | 9 | 14.30 |
| Signaling | | | 6976; Alsterpaullone; | PRKCA; MAPK8; | | |
| Pathway | | | Vandetanib; H-7 | CDK6; AKT1; | | |
| Netpath | | | dihydrochloride; sotrastaurin; | PRKCB; GSK3B; | | |
| | | | C-1; SP600125; H 89 | MAPK1 | | |
| | | | dihydrochloride; APIGENIN | | | |
| TNF alpha | 9 | 93 | AT7867; SC-1; TW- | BCL2L1; RAF1; | 9 | 13.71 |
| Signaling | | | 37; SP600125; rigosertib; H 89 | PLK1; MAPK9; | | |
| Pathway | | | dihydrochloride; ABT- | MAPK3; CSNK2A1; | | |
| | | | 737; Sorafenib; APIGENIN | MAPK8; AKT1; | | |
| | | | | MAPK1 | | |
| Wnt | 8 | 66 | Y-27632; Go | PRKCE; MAPK9; | 9 | 11.58 |
| Signaling | | | 6976; Alsterpaullone; H-7 | PRKCA; PRKCD; | | |
| Pathway | | | dihydrochloride; sotrastaurin; | PRKCQ; PRKCH; | | |
| | | | C-1; SP600125; APIGENIN | GSK3B; PRKD1; | | |
| | | | | MAPK10 | | |
| Cardiac | 7 | 54 | AT7867; CDK9 inhibitor 14; H | RAF1; HDAC4; | 9 | 11.34 |
| Hypertrophic | | | 89 dihydrochloride; SCH727965; | CDK9; AKT2; | | |
| Response | | | Belinostat; Sorafenib; | HDAC9; AKT1; | | |
| | | | Pandacostat | HDAC5; CDK7; | | |
| | | | | HDAC7 | | |
| GPCRs, | 6 | 103 | AZELASTINE | HTR2A; CHRM2; | 9 | 9.64 |
| Other | | | HYDROCHLORIDE; SURAMIN; | HRH4; ADORA2A; | | |
| | | | Purmorphamine; NVP- | P2RY11; SMO; | | |
| | | | LDE225; CV 1808; Doxepine | P2RY13; CHRM3; | | |
| | | | HCl | ADRA1D | | |
| Notch | 21 | 61 | Bosutinib; AT7867; Bosutinib; | HDAC1; LCK; | 8 | 14.04 |
| Signaling | | | VX-680; 1-Naphthyl | AKT1; JAK2; | | |
| Pathway | | | PP1; apicidin; Cyt387; Tozasertib; | EP300; GSK3B; | | |
| | | | Alsterpaullone; TG- | SRC; HDAC2 | | |
| | | | 101348; INCB018424; Vandetanib; | | | |
| | | | Tozasertib VX-680 (MK- | | | |
| | | | 0457); KX2- | | | |
| | | | 391; INCB018424; Dasatinib; | | | |
| | | | AZD1480; (−)-Epigallocatechin | | | |
| | | | Gallate; H 89 | | | |
| | | | dihydrochloride; Belinostat; | | | |
| | | | Pandacostat | | | |
| Signaling of | 13 | 34 | Bosutinib; SC-1; Bosutinib; PF- | RAF1; MAPK3; | 8 | 17.05 |
| Hepatocyte | | | 573228; VX-680; 1-Naphthyl | MAP2K2; PTK2; | | |
| Growth | | | PP1; Tozasertib; Vandetanib; | MAPK8; MAP2K1; | | |
| Factor | | | Tozasertib VX-680 (MK- | SRC; MAPK1 | | |
| Receptor | | | 0457); SP600125; KX2- | | | |
| | | | 391; Dasatinib; Sorafenib | | | |
| Nuclear | 10 | 33 | TTNPB; AM- | ABCB1; PPARD; | 8 | 8.26 |
| Receptors | | | 580; PODOPHYLLIN | NR1I2; RARG; | | |
| in Lipid | | | ACETATE; Erlotinib; 4-(4- | RARA; RARB; | | |
| Metabolism | | | octylphenyl)benzoate; TTNPB; | CYP3A4; CYP2C9 | | |
| and | | | NP-009852; NP- | | | |
| Toxicity | | | 009832; FLAVONE; | | | |
| | | | ERYTHROMYCIN STEARATE; | | | |
| | | | Retinoic acid | | | |
| TWEAK | 8 | 42 | AT7867; SC- | HDAC1; MAPK9; | 8 | 15.15 |
| Signaling | | | 1; apicidin; Alsterpaullone; SP6 | MAPK3; AKT2; | | |
| Pathway | | | 00125; H 89 | MAPK8; AKT1; | | |
| | | | dihydrochloride; Belinostat; | GSK3B; MAPK1 | | |
| | | | Pandacostat | | | |
| MAPK | 7 | 29 | Bosutinib; SC- | RAF1; MAPK3; | 8 | 18.11 |
| Cascade | | | 1; Bosutinib; RAF265; SP600125; | MAP2K2; MAP3K2; | | |
| | | | Sorafenib; APIGENIN | BRAF; MAP2K1; | | |
| | | | | MAPK10; MAPK1 | | |
| Nuclear | 6 | 38 | TTNPB; AM-580; Erlotinib; 4- | PPARD; NR1I2; | 8 | 8.64 |
| Receptors | | | (4-octylphenyl)benzoate; TTNPB; | RARG; RARA; | | |
| | | | Retinoic acid | RARB; RXRA; | | |
| | | | | RXRB; NR2C2 | | |
| IL-1 | 6 | 54 | Bosutinib; AT7867; SC- | MAPK9; MAPK3; | 8 | 24.34 |
| signaling | | | 1; Bosutinib; SP600125; H 89 | MAP2K2; MAP3K2; | | |
| pathway | | | dihydrochloride | MAPK8; MAP2K1; | | |
| | | | | AKT1; MAPK1 | | |
| FSH | 21 | 27 | Bosutinib; AT7867; SC- | RAF1; PRKCA; | 7 | 14.54 |
| signaling | | | 1; Bosutinib; VX-680; 1- | MAPK3; PRKACA; | | |
| pathway | | | Naphthyl PP1; Go | AKT1; SRC; MAPK1 | | |
| | | | 6976; Tozasertib; Vandetanib; | | | |
| | | | Tozasertib VX-680 (MK- | | | |
| | | | 0457); H-7 | | | |
| | | | dihydrochloride; sotrastaurin; | | | |
| | | | C-1; KX2- | | | |
| | | | 391; Dasatinib; “Dibutyryl- | | | |
| | | | cAMP, sodium salt”; HA- | | | |
| | | | 1077; H 89 | | | |
| | | | dihydrochloride; HA- | | | |
| | | | 1077; Phorbol 12-Myristate | | | |
| | | | 13-Acetate; Sorafenib | | | |
| Alpha 6 | 17 | 31 | Bosutinib; AT7867; SC- | PRKCA; MAPK3; | 7 | 17.19 |
| Beta 4 | | | 1; Bosutinib; PF-573228; VX- | PRKCD; PTK2; | | |
| signaling | | | 680; 1-Naphthyl PP1; Go | AKT1; SRC; MAPK1 | | |
| pathway | | | 6976; Tozasertib; Vandetanib; | | | |
| | | | Tozasertib VX-680 (MK- | | | |
| | | | 0457); H-7 | | | |
| | | | dihydrochloride; sotrastaurin; | | | |
| | | | C-1; KX2-391; Dasatinib; H 89 | | | |
| | | | dihydrochloride | | | |
| Nicotine | 15 | 21 | Alsterpaullone; H-7 | ADCY2; CDK5; | 7 | 8.03 |
| Activity on | | | dihydrochloride; C- | DRD2; PRKACA; | | |
| Dopaminergic | | | 1; BARBITAL; “Dibutyryl-cAMP, | CHRNA4; CHRNB2; | | |
| Neurons | | | sodium salt”; N9- | CHRNA3 | | |
| | | | isopropylolomoucine; HA- | | | |
| | | | 1077; H 89 | | | |
| | | | dihydrochloride; HA- | | | |
| | | | 1077; Phorbol 12-Myristate | | | |
| | | | 13-Acetate; UB 165 | | | |
| | | | fumarate; SCH727965; Doxepine | | | |
| | | | HCl; APIGENIN; COLFORSIN | | | |
| miRs in | 10 | 18 | Y-27632; Go 6976; H-7 | PRKCE; PRKCA; | 7 | 10.02 |
| Muscle Cell | | | dihydrochloride; sotrastaurin; | PRKCD; PRKACA; | | |
| Differentiation | | | C-1; “Dibutyryl-cAMP, sodium | PRKCQ; PRKCH; | | |
| | | | salt”; HA-1077; H 89 | PRKD1 | | |
| | | | dihydrochloride; HA- | | | |
| | | | 1077; Phorbol 12-Myristate | | | |
| | | | 13-Acetate | | | |
| TGF Beta | 9 | 54 | SC-1; Cyt387; TG- | TGFBR2; MAPK9; | 7 | 11.85 |
| Signaling | | | 101348; INCB018424; SP600125; | MAPK3; TGFBR1; | | |
| Pathway | | | INCB018424; AZD1480; (−)- | EP300; JAK1; | | |
| | | | Epigallocatechin Gallate; LY | SERPINE1 | | |
| | | | 364947 | | | |
| G1 to S cell | 8 | 69 | Arcyriaflavin | CDK4; CDK1; | 7 | 9.35 |
| cycle | | | A; Alsterpaullone; CDK9 | CDK6; WEE1; | | |
| control | | | inhibitor 14; N9- | CDK7; ATM; | | |
| | | | isopropylolomoucine; MK- | CDK2 | | |
| | | | 1775; KU- | | | |
| | | | 55933; SCH727965; APIGENIN | | | |
| Serotonin | 6 | 19 | Bosutinib; SC- | HTR2A; RAF1; | 7 | 19.12 |
| Receptor 2 | | | 1; Bosutinib; Eltoprazine | HTR2C; MAPK3; | | |
| and ELK- | | | hydrochloride; Doxepine | MAP2K2; MAP2K1; | | |
| SRF/GATA4 | | | HCl; Sorafenib | HTR2B | | |
| signaling | | | | | | |
| Parkin- | 5 | 71 | VER155008; Colchicine; | TUBA1A; TUBA4A; | 7 | 8.51 |
| Ubiquitin | | | Vinblastine | TUBB; HSPA1B; | | |
| Proteasomal | | | sulfate; PODOFILOX; | TUBA1B; TUBB1; | | |
| System | | | COLCHICINE | HSPA1A | | |
| pathway | | | | | | |
| Apoptosis | 11 | 84 | AT7867; TW-37; BMS- | BCL2; BCL2L1; | 6 | 10.65 |
| | | | 536924; SP600125; PODOFILOX; | BCL2L2; AKT1; | | |
| | | | H 89 dihydrochloride; ABT- | IGF1R; MAPK10 | | |
| | | | 737; ABT-199 (GDC-0199); | | | |
| | | | APIGENIN; NVP- | | | |
| | | | ADW742; OSI-906 (Linsitinib) | | | |
| IL17 | 9 | 31 | AT7867; SC- | MAPK3; AKT1; | 6 | 15.37 |
| signaling | | | 1; Cyt387; Alsterpaullone; TG- | JAK2; GSK3B; | | |
| pathway | | | 101348; INCB018424; | JAK1; MAPK1 | | |
| | | | INCB018424; AZD1480; H 89 | | | |
| | | | dihydrochloride | | | |
| Extracellular | 9 | 31 | AT7867; Neratinib; Vandetanib; | TGFBR2; RAF1; | 6 | 12.01 |
| vesicle- | | | Erlotinib; Erlotinib; Gefitinib; | EGFR; AKT1; | | |
| mediated | | | H 89 dihydrochloride; Sorafenib; | TGFBR1; ERBB2 | | |
| signaling in | | | LY 364947 | | | |
| recipient | | | | | | |
| cells | | | | | | |
| IL-9 | 8 | 17 | Bosutinib; SC- | MAPK3; MAP2K2; | 6 | 18.38 |
| Signaling | | | 1; Bosutinib; Cyt387; TG- | MAP2K1; JAK3; | | |
| Pathway | | | 101348; INCB018424; | JAK1; MAPK1 | | |
| | | | INCB018424; AZD1480 | | | |
| ErbB | 16 | 55 | Bosutinib; AT7867; Bosutinib; | ERBB4; PRKCA; | 5 | 15.95 |
| Signaling | | | VX-680; 1-Naphthyl | SRC; AKT3; | | |
| Pathway | | | PP1; Neratinib; Go | ERBB2 | | |
| | | | 6976; Tozasertib; Vandetanib; | | | |
| | | | Tozasertib VX-680 (MK- | | | |
| | | | 0457); H-7 | | | |
| | | | dihydrochloride; sotrastaurin; | | | |
| | | | C-1; KX2- | | | |
| | | | 391; Erlotinib; Dasatinib | | | |
| Apoptosis- | 12 | 53 | Bosutinib; AT7867; SC- | HDAC1; ABL1; | 5 | 19.29 |
| related | | | 1; Bosutinib; PF-573228; KW | PTK2; AKT1; | | |
| network | | | 2449; 1-Naphthyl | MAPK1 | | |
| due to | | | PP1; apicidin; Dasatinib; H 89 | | | |
| altered | | | dihydrochloride; Belinostat; | | | |
| Notch3 in | | | Pandacostat | | | |
| ovarian | | | | | | |
| cancer | | | | | | |
| G13 | 10 | 38 | Bosutinib; Bosutinib; Y- | PIK3CD; ROCK1; | 5 | 13.72 |
| Signaling | | | 27632; HA- | TNK2; MAPK10; | | |
| Pathway | | | 1077; SP600125; HA-1077; H | ROCK2 | | |
| | | | 89 dihydrochloride; HA- | | | |
| | | | 1077; APIGENIN; CAL-101 | | | |
| Interferon | 10 | 58 | Bosutinib; Bosutinib; 1- | PIK3CD; TYK2; | 5 | 14.75 |
| type I | | | Naphthyl PP1; Cyt387; TG- | JAK1; FYN; | | |
| signaling | | | 101348; INCB018424; | RPS6KA5 | | |
| pathways | | | INCB018424; AZD 1480; H 89 | | | |
| | | | dihydrochloride; CAL-101 | | | |
| Endochondral | 9 | 66 | Dorsomorphin | HDAC4; DDR2; | 5 | 8.01 |
| Ossification | | | dihydrochloride; Sunitinib | IGF1R; BMPR1A; | | |
| | | | malate; BMS- | FGFR1 | | |
| | | | 536924; PODOFILOX; Belinostat; | | | |
| | | | Sorafenib; NVP- | | | |
| | | | ADW742; Pandacostat; OSI- | | | |
| | | | 906 (Linsitinib) | | | |
| Serotonin | 6 | 18 | Bosutinib; SC- | MAPK3; MAP2K2; | 5 | 19.57 |
| Receptor | | | 1; Bosutinib; RAF265; H 89 | BRAF; MAP2K1; | | |
| 4/6/7 and | | | dihydrochloride; Sorafenib | RPS6KA5 | | |
| NR3C | | | | | | |
| Signaling | | | | | | |
| Adipogenesis | 6 | 131 | TTNPB; AM-580; TTNPB; (−)- | AHR; PPARD; | 5 | 8.26 |
| | | | Epigallocatechin | RARA; RXRA; | | |
| | | | Gallate; Stem Regenin | SERPINE1 | | |
| | | | 1; Retinoic acid | | | |
| Monoamine | 5 | 32 | AZELASTINE | SLC6A4; SLC6A2; | 5 | 11.54 |
| Transport | | | HYDROCHLORIDE; OXOLINIC | ADORA2A; HRH3; | | |
| | | | ACID; AMINOBENZTROPINE; | SLC6A3 | | |
| | | | CV 1808; Doxepine HCl | | | |
| Signal | 3 | 25 | AT7867; SC-1; H 89 | MAPK3; AKT2; | 5 | 24.11 |
| Transduction | | | dihydrochloride | AKT1; AKT3; | | |
| of SIP | | | | MAPK1 | | |
| Receptor | | | | | | |
| Aryl | 14 | 43 | Arcyriaflavin | AHR; EGFR; | 4 | 10.09 |
| Hydrocarbon | | | A; Neratinib; Sunitinib | RET; CDK2 | | |
| Receptor | | | Malate; TG- | | | |
| | | | 101348; Vandetanib; Erlotinib; | | | |
| | | | Sunitinib malate; CDK9 | | | |
| | | | inhibitor 14; Erlotinib; N9- | | | |
| | | | isopropylolomoucine; Gefitinib; | | | |
| | | | StemRegenin | | | |
| | | | 1; SCH727965; Sorafenib | | | |
| Bladder | 9 | 29 | Arcyriaflavin | CDK4; BRAF; | 4 | 10.50 |
| Cancer | | | A; Neratinib; Vandetanib; | EGFR; ERBB2 | | |
| | | | Erlotinib; RAF265; CDK9 inhibitor | | | |
| | | | 14; Erlotinib; Gefitinib; Sorafenib | | | |
| T-Cell | 9 | 30 | Bosutinib; AT7867; Bosutinib; | LCK; AKT1; | 4 | 19.74 |
| Receptor | | | VX-680; 1-Naphthyl | GSK3B; FYN | | |
| and Co- | | | PP1; Tozasertib; Alsterpaullone; | | | |
| stimulatory | | | Tozasertib VX-680 (MK- | | | |
| Signaling | | | 0457); H 89 dihydrochloride | | | |
| Type II | 8 | 41 | Cyt387; TG- | EIF2AK2; PRKCD; | 4 | 10.25 |
| interferon | | | 101348; INCB018424; sotrastaurin; | JAK2; JAK1 | | |
| signaling | | | C-1; INCB018424; “7- | | | |
| (IFNG) | | | DESACETOXY-6,7- | | | |
| | | | DEHYDROGEDUNIN”; AZD1480 | | | |
| Constitutive | 5 | 20 | PODOPHYLLIN ACETATE; NP- | ABCB1; RXRA; | 4 | 7.52 |
| Androstane | | | 009852; NP- | CYP3A4; CYP2C9 | | |
| Receptor | | | 009832; FLAVONE; | | | |
| Pathway | | | ERYTHROMYCIN STEARATE; | | | |
| | | | Retinoic acid | | | |
| DNA | 4 | 61 | Alsterpaullone; SP600125; | PIK3CD; MAPK9; | 4 | 8.85 |
| Damage | | | APIGENIN; CAL-101 | GSK3B; MAPK10 | | |
| Response | | | | | | |
| (only ATM | | | | | | |
| dependent) | | | | | | |
| Peptide | 3 | 73 | BNTX maleate; “7,4′- | OPRD1; SSTR4; | 4 | 7.39 |
| GPCRs | | | DIHYDROXYFLAVONE”; “L- | OPRK1; OPRM1 | | |
| | | | 803,087 trifluoroacetate” | | | |
| EPO | 15 | 27 | Bosutinib; Bosutinib; VX- | RAF1; JAK2; SRC | 3 | 14.17 |
| Receptor | | | 680; 1-Naphthyl | | | |
| Signaling | | | PP1; Cyt387; Tozasertib; TG- | | | |
| | | | 101348; INCB018424; Vandetanib; | | | |
| | | | Tozasertib VX-680 (MK- | | | |
| | | | 0457); KX2- | | | |
| | | | 391; INCB018424; Dasatinib; | | | |
| | | | AZD1480; Sorafenib | | | |
| miRNAs | 7 | 15 | Bosutinib; Bosutinib; KW | ABL1; CDK6; ATM | 3 | 17.68 |
| involved in | | | 2449; 1-Naphthyl | | | |
| DNA | | | PP1; Dasatinib; KU- | | | |
| damage | | | 55933; APIGENIN | | | |
| response | | | | | | |
| Integrated | 7 | 12 | AT7867; Sunitinib | AKT1; EP300; | 3 | 12.95 |
| Pancreatic | | | Malate; Sunitinib | PDGFRA | | |
| Cancer | | | malate; AZD2171; Ki8751; (−)- | | | |
| Pathway | | | Epigallocatechin Gallate; H 89 | | | |
| | | | dihydrochloride | | | |
| Amyotrophic | 4 | 34 | TW-37; ABT-737; ABT-199 | PPP3CA; BCL2; | 3 | 8.38 |
| lateral | | | (GDC-0199); cyclosporine | BCL2L1 | | |
| sclerosis | | | | | | |
| (ALS) | | | | | | |
| Ovarian | 4 | 31 | Arcyriaflavin A; Dorsomorphin | CDK4; ATM; | 3 | 11.00 |
| Infertility | | | dihydrochloride; CDK9 | BMPR1B | | |
| Genes | | | inhibitor 14; KU-55933 | | | |
| Fluoropyrimidine | 3 | 33 | Ko-143; Acyclovir; zebularine | CDA; ABCG2; TK1 | 3 | 7.74 |
| Activity | | | | | | |
| p38 MAPK | 3 | 34 | CGP57380; H89 | MKNK1; TGFBR1; | 3 | 7.42 |
| Signaling | | | dihydrochloride; LY 364947 | RPS6KA5 | | |
| Pathway | | | | | | |
| Eicosanoid | 3 | 19 | SURAMIN; valdecoxib; | PLA2G2A; DPEP1; | 3 | 9.43 |
| Synthesis | | | Cilastatin sodium | PTGS2 | | |
| Drug | 3 | 17 | PODOPHYLLIN | ABCB1; NR1I2; | 3 | 8.35 |
| Induction | | | ACETATE; Erlotinib; | CYP3A4 | | |
| of Bile Acid | | | ERYTHROMYCIN STEARATE | | | |
| Pathway | | | | | | |
| Nucleotide | 2 | 11 | SURAMIN; CV 1808 | P2RY2; ADORA2A; | 3 | 9.28 |
| GPCRs | | | | P2RY1 | | |
| Glycolysis | 1 | 48 | LONIDAMINE | HK3; HK2; HK1 | 3 | 6.13 |
| and | | | | | | |
| Gluconeogenesis | | | | | | |
| TCA Cycle | 10 | 5 | Neratinib; Cyt387; TG- | EGFR; JAK1 | 2 | 10.60 |
| Nutrient | | | 101348; INCB018424; Vandetanib; | | | |
| Utilization | | | Erlotinib; INCB018424; Erlotinib; | | | |
| and | | | AZD1480; Gefitinib | | | |
| Invasiveness | | | | | | |
| of | | | | | | |
| Ovarian | | | | | | |
| Cancer | | | | | | |
| Gastric | 9 | 31 | Neratinib; Daunorubicin; | TOP2A; EGFR | 2 | 9.81 |
| cancer | | | Vandetanib; Erlotinib; Erlotinib; | | | |
| network 2 | | | PODOFILOX; Bisantrene | | | |
| | | | dihydrochloride; Gefitinib; | | | |
| | | | Doxorubicin | | | |
| Gastric | 9 | 29 | KW 2449; VX- | AURKA; TOP2A | 2 | 11.59 |
| Cancer | | | 680; Tozasertib; Daunorubicin; | | | |
| Network 1 | | | Tozasertib VX-680 (MK- | | | |
| | | | 0457); PODOFILOX; Bisantrene | | | |
| | | | dihydrochloride; Doxorubicin; | | | |
| | | | MLN8237 | | | |
| Cholesterol | 7 | 15 | Cerivastatin; Cerivastatin; | HMGCR; CYP51A1 | 2 | 8.06 |
| Biosynthesis | | | TIOCONAZOLE; Pitavastatin | | | |
| | | | calcium; ERYTHROMYCIN | | | |
| | | | STEARATE; lovastatin; fluvastatin | | | |
| Serotonin | 6 | 4 | Cyt387; TG- | HTR2A; JAK2 | 2 | 10.06 |
| Receptor 2 | | | 101348; INCB018424; INCB018424; | | | |
| and STAT3 | | | AZD1480; Doxepine HCl | | | |
| Signaling | | | | | | |
| Type III | 5 | 10 | Cyt387; TG- | TYK2; JAK1 | 2 | 10.70 |
| interferon | | | 101348; INCB018424; INCB018424; | | | |
| signaling | | | AZD1480 | | | |
| Osteoblast | 5 | 17 | Sunitinib Malate; Sunitinib | PDGFRB; PDGFRA | 2 | 9.48 |
| Signaling | | | malate; AZD2171; Ki8751; | | | |
| | | | Sorafenib | | | |
| FAS | 4 | 42 | TW-37; SP600125; ABT- | BCL2; MAPK8 | 2 | 9.24 |
| pathway | | | 737; ABT-199 (GDC-0199) | | | |
| and Stress | | | | | | |
| induction | | | | | | |
| of HSP | | | | | | |
| regulation | | | | | | |
| Tryptophan | 3 | 46 | PODOPHYLLIN | CYP3A4; ALDH2 | 2 | 7.49 |
| metabolism | | | ACETATE; ERYTHROMYCIN | | | |
| | | | STEARATE; tetraethylthiuram | | | |
| | | | disulfide | | | |
| Irinotecan | 3 | 14 | Ko-143; PODOPHYLLIN | ABCG2; CYP3A4 | 2 | 8.50 |
| Pathway | | | ACETATE; ERYTHROMYCIN | | | |
| | | | STEARATE | | | |
| Fatty Acid | 3 | 15 | PODOPHYLLIN | CYP3A4; ALDH2 | 2 | 7.49 |
| Omega | | | ACETATE; ERYTHROMYCIN | | | |
| Oxidation | | | STEARATE; tetraethylthiuram | | | |
| | | | disulfide | | | |
| Farnesoid | 3 | 19 | PODOPHYLLIN | RXRA; CYP3A4 | 2 | 7.63 |
| X Receptor | | | ACETATE; ERYTHROMYCIN | | | |
| Pathway | | | STEARATE; Retinoic acid | | | |
| Selenium | 3 | 86 | BMS-536924; ERYTHROMYCIN | INSR; ALB | 2 | 7.78 |
| Micronutrient | | | STEARATE; OSI-906 (Linsitinib) | | | |
| Network | | | | | | |
| Apoptosis | 3 | 18 | SP600125; VER155008; | MAPK10; HSPA1A | 2 | 8.57 |
| Modulation | | | APIGENIN | | | |
| by HSP70 | | | | | | |
| Folate | 3 | 67 | BMS-536924; ERYTHROMYCIN | INSR; ALB | 2 | 7.78 |
| Metabolism | | | STEARATE; OSI-906 (Linsitinib) | | | |
| Liver X | 3 | 10 | PODOPHYLLIN | RXRA; CYP3A4 | 2 | 7.63 |
| Receptor | | | ACETATE; ERYTHROMYCIN | | | |
| Pathway | | | STEARATE; Retinoic acid | | | |
| Vitamin | 3 | 52 | BMS-536924; ERYTHROMYCIN | INSR; ALB | 2 | 7.78 |
| B12 | | | STEARATE; OSI-906 (Linsitinib) | | | |
| Metabolism | | | | | | |
| Secretion | 2 | 4 | AZELASTINE | HRH2; CHRM1 | 2 | 11.02 |
| of | | | HYDROCHLORIDE; Doxepine | | | |
| Hydrochloric | | | HCl | | | |
| Acid in | | | | | | |
| Parietal | | | | | | |
| Cells | | | | | | |
| Apoptosis | 2 | 13 | Go 6976; VER155008 | PRKD1; HSPA1A | 2 | 11.09 |
| Modulation | | | | | | |
| and | | | | | | |
| Signaling | | | | | | |
| Heart | 2 | 44 | SC-1; Dorsomorphin | BMPR1A; MAPK1 | 2 | 20.40 |
| Development | | | dihydrochloride | | | |
| TFs | 2 | 8 | AT7867; H 89 dihydrochloride | AKT2; AKT1 | 2 | 21.31 |
| Regulate | | | | | | |
| miRNAs | | | | | | |
| related to | | | | | | |
| cardiac | | | | | | |
| hypertrophy | | | | | | |
| Nicotine | 2 | 4 | ETHOSUXIMIDE; UB 165 | CACNA1G; CHRNA3 | 2 | 8.15 |
| Activity on | | | fumarate | | | |
| Chromaffin | | | | | | |
| Cells | | | | | | |
| Codeine | 2 | 8 | PODOPHYLLIN | ABCB1; CYP3A4 | 2 | 8.02 |
| and | | | ACETATE; ERYTHROMYCIN | | | |
| Morphine | | | STEARATE | | | |
| Metabolism | | | | | | |
| Hypothetical | 2 | 33 | BARBITAL; Doxepine HCl | DRD2; GRIA2 | 2 | 7.75 |
| Network | | | | | | |
| for Drug | | | | | | |
| Addiction | | | | | | |
| Vitamin D | 1 | 10 | Retinoic acid | RXRA; RXRB | 2 | 6.86 |
| Metabolism | | | | | | |
| PDGF | 1 | 12 | SC-1 | MAPK3; MAPK1 | 2 | 25.00 |
| Pathway | | | | | | |
| Dopamine | 7 | 13 | H-7 dihydrochloride; C- | PRKACA | 1 | 8.23 |
| metabolism | | | 1; “Dibutyryl-cAMP, sodium | | | |
| | | | salt”; HA-1077; H 89 | | | |
| | | | dihydrochloride; HA- | | | |
| | | | 1077; Phorbol 12-Myristate | | | |
| | | | 13-Acetate | | | |
| Statin | 5 | 29 | Cerivastatin; Cerivastatin; | HMGCR | 1 | 8.12 |
| Pathway | | | Pitavastatin | | | |
| | | | calcium; lovastatin; fluvastatin | | | |
| TP53 | 5 | 13 | Bosutinib; Bosutinib; KW | ABL1 | 1 | 21.90 |
| Network | | | 2449; 1-Naphthyl | | | |
| | | | PP1; Dasatinib | | | |
| ID signaling | 4 | 16 | Arcyriaflavin A; CDK9 inhibitor | CDK2 | 1 | 10.10 |
| pathway | | | 14; N9- | | | |
| | | | isopropylolomoucine; SCH727965 | | | |
| DNA | 4 | 41 | Arcyriaflavin A; CDK9 inhibitor | CDK2 | 1 | 10.10 |
| Replication | | | 14; N9- | | | |
| | | | isopropylolomoucine; SCH727965 | | | |
| Inflammatory | 3 | 30 | VX-680; Tozasertib; Tozasertib | LCK | 1 | 13.99 |
| Response | | | VX-680 (MK-0457) | | | |
| Pathway | | | | | | |
| Influenza A | 3 | 12 | TW-37; ABT-737; ABT-199 | BCL2 | 1 | 9.05 |
| virus | | | (GDC-0199) | | | |
| infection | | | | | | |
| Ectoderm | 2 | 10 | Ro 31-8220 | PIM1 | 1 | 10.67 |
| Differentiation | | | mesylate; APIGENIN | | | |
| Oxidative | 2 | 29 | SP600125; APIGENIN | MAPK10 | 1 | 8.26 |
| Stress | | | | | | |
| Arachidonate | 2 | 5 | NP-009852; NP- | CYP2C9 | 1 | 7.36 |
| Epoxygenase/ | | | 009832; FLAVONE | | | |
| Epoxide | | | | | | |
| Hydrolase | | | | | | |
| Hedgehog | 2 | 16 | Purmorphamine; NVP-LDE225 | SMO | 1 | 8.61 |
| Signaling | | | | | | |
| Pathway | | | | | | |
| Mitochondrial | 1 | 19 | cyclosporine | PPP3CA | 1 | 6.35 |
| Gene | | | | | | |
| Expression | | | | | | |
| Spinal Cord | 1 | 4 | valdecoxib | PTGS2 | 1 | 8.60 |
| Injury | | | | | | |
| Proteasome | 1 | 61 | MLN2238 | PSMB5 | 1 | 7.13 |
| Degradation | | | | | | |
| ACE | 1 | 17 | PERINDOPRIL ERBUMINE | ACE | 1 | 7.27 |
| Inhibitor | | | | | | |
| Pathway | | | | | | |
| Cytoplasmic | 1 | 88 | H 89 dihydrochloride | RPS6KA3 | 1 | 7.14 |
| Ribosomal | | | | | | |
| Proteins | | | | | | |
| Parkinsons | 1 | 71 | LRRK2-IN-1 | LRRK2 | 1 | 8.88 |
| Disease | | | | | | |
| Pathway | | | | | | |
| Matrix | 1 | 30 | (−)-Epigallocatechin Gallate | MMP7 | 1 | 7.36 |
| Metalloproteinases | | | | | | |
| Electron | 1 | 103 | oligomycin A | ATP6 | 1 | 7.16 |
| Transport | | | | | | |
| Chain | | | | | | |
| Glycogen | 1 | 36 | Alsterpaullone | GSK3B | 1 | 12.55 |
| Metabolism | | | | | | |
| Blood | 1 | 25 | (−)-Epigallocatechin Gallate | SERPINE1 | 1 | 7.36 |
| Clotting | | | | | | |
| Cascade | | | | | | |
| SREBF and | 1 | 4 | Dorsomorphin | PRKAA1 | 1 | 11.09 |
| miR33 in | | | dihydrochloride | | | |
| cholesterol | | | | | | |
| and lipid | | | | | | |
| homeostasis | | | | | | |
| Integrated | 1 | 17 | CDK9 inhibitor 14 | CDK7 | 1 | 9.91 |
| Breast | | | | | | |
| Cancer | | | | | | |
| Pathway | | | | | | |
| Complement | 1 | 60 | (−)-Epigallocatechin Gallate | SERPINE1 | 1 | 7.36 |
| and | | | | | | |
| Coagulation | | | | | | |
| Cascades | | | | | | |
| Translation | 1 | 50 | 7-DESACETOXY-6,7- | EIF2AK2 | 1 | 7.92 |
| Factors | | | DEHYDROGEDUNIN | | | |
| Oxidative | 1 | 60 | oligomycin A | ATP6 | 1 | 7.16 |
| phosphorylation | | | | | | |
| Eukaryotic | 1 | 41 | CDK9 inhibitor 14 | CDK7 | 1 | 9.91 |
| Transcription | | | | | | |
| Initiation | | | | | | |
| Prostaglandin | 1 | 31 | valdecoxib | PTGS2 | 1 | 8.60 |
| Synthesis | | | | | | |
| and | | | | | | |
| Regulation | | | | | | |
| Serotonin | 1 | 11 | Doxepine HCl | SLC6A4 | 1 | 6.87 |
| Transporter | | | | | | |
| Activity | | | | | | |

### Example 3: Validation of Selected Compounds and Pathways

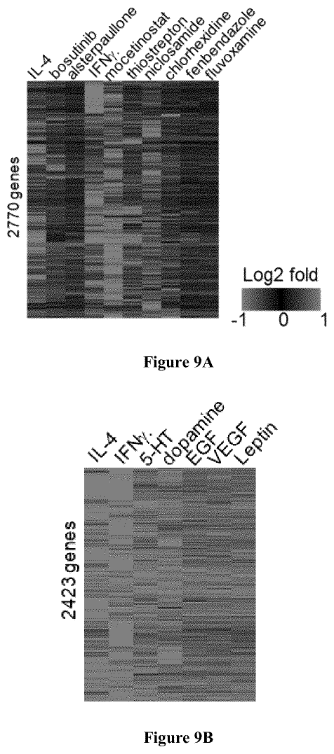

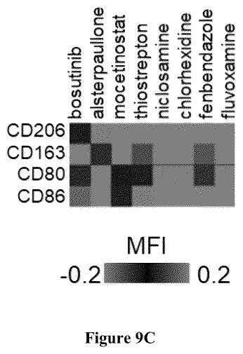

To validate the effect of identified compounds on macrophage activation, we assayed dosage responses of the commercially available top list of compounds to determine their effective concentration (EC) on cell shape change. 20 of 23 selected M1-activating and 4 of 6 M2-activating compounds showed strong dosage effects with an EC below 10 M (FIGS. 2A-2B and Table 2). We performed RNA-seq analysis with 6 M1-activating (mocetinostat, thiostrepton, niclosamide, chlorhexidine, fenbendazole and fluvoxamine) and 2 M2-activating (bosutinib and alsterpaullone) compounds to determine if they activate macrophages at the transcriptional level. Fresh hMDMs were treated with compounds at the EC for 24 hours, followed by RNA-seq. The compounds induced diverse transcriptional responses with variable number of differentially expressed genes (DEG) to similar degrees as those induced by IL-4 or IFNγ (FIG. 9A). To explore the functional differences of hMDMs induced by compounds, gene set enrichment analysis (GSEA) of transcriptional responses to each compound was compared to previously identified 49 gene expression modules in response to 29 different stimuli in hMDMs. Similar to IFNγ, the six M1-activating compounds up-regulated the gene expression of typical M1 modules (module #7, #8) induced by IFNγ, as well as chronic inflammation TPP modules (module #30, #32) induced by TNFα/PGE2/P3C (FIG. 2C). The M1-activating compounds also down-regulated the modules (#26, #27) similarly as LPS. The two M2-activating compounds down-regulated the gene expression of typical M1 modules although they did not upregulate the gene expression modules (module #15) induced by IL-4 (FIG. 2C). Consistently, all M1-activating compound upregulated expression of classical M1 markers CD80 and CD86 and down-regulated expression of classical M2 markers CD163 and CD206. Both M2-activating compounds down-regulated M1 markers (FIG. 9C). Moreover, based on function enrichment analysis of the DEGs, all 8 compounds induced consensus pathways related to inflammatory response, chemotaxis/chemokine-mediated signaling and response to IFNγ and TNFα (FIG. 2D). These results suggest that select compounds modulate macrophage activation at the transcriptional levels.

We also analyzed transcriptional responses of hMDMs to ligands of novel pathways, including serotonin (5HT), dopamine, VEGF, EGF and leptin by RNA-seq. Each ligand induced diverse transcriptional response (FIG. 9B). In particular, 5HT, VEGF, EGF and leptin up-regulated the gene expression of typical M1 modules (#7, #8) but down-regulated the gene expression of the TPP modules (#30, #32) (FIG. 2E). In contrast, dopamine down-regulated the gene expression of typical M1 modules but up-regulated the TPP modules (FIG. 2E), suggesting these ligands regulate different aspects of macrophage activation. Function enrichment analysis of the DEGs identified induction of pathways related to inflammatory response, chemotaxis/chemokine-mediated signaling and wound healing by these ligands (FIG. 2F). Taken together, these results suggest that the compounds as well as upstream signals of their protein targets modulate macrophage activation.

Table 2 shows dosage information of selected compounds on M0 macrophages.

| | | | Max | |
|---|---|---|---|---|
| | | | absolute | |
| Compound | EC | R square | Z-value | Category |
| Taxol | 0.10459189 | 0.416 | −6.68139 | M1 |
| Cucurbitacin I | 0.34174063 | 0.6048 | −4.808 | M1 |
| Chlorhexidine | 1.62736296 | 0.7308 | −13.45 | M1 |
| Fenbendazole | 0.4703644 | 0.8408 | −12.37 | M1 |
| Thiostrepton | 3.93691593 | 0.542 | −12.75 | M1 |
| Diphenyleneiodonium | 0.67677365 | 0.4593 | −8.509 | M1 |
| chloride | | | | |
| LE135 | 0.41887689 | 0.5289 | −8.912 | M1 |
| Fluvoxamine | 10.6420447 | 0.8318 | −7.38 | M1 |
| Niclosamide | 0.79658547 | 0.8077 | −8.929 | M1 |
| MS275 | 1.04687704 | 0.5386 | −7.993 | M1 |
| Mocetinostat | 0.45359214 | 0.7105 | −15.14 | M1 |
| Pimozide | 1.17941118 | 0.2042 | −8.006 | M1 |
| NP-010176 | 4.03386043 | 0.5479 | −8.156 | M1 |
| HMN214 | 0.10382862 | 0.6514 | −12.51 | M1 |
| Celastrol | 0.79618903 | 0.4416 | −4.485 | M1 |
| Cantharidin | 0.17983226 | 0.2334 | −31.36 | M1 |
| NVP 231 | 2.24851869 | 0.8661 | −22.37 | M1 |
| FTY720 | 2.94456442 | 0.8244 | −11.58 | M1 |
| Evodiamine | 0.3969275 | 0.9413 | −26.36 | M1 |
| Penfluridol | 2.52355815 | 0.8439 | −4.873 | M1 |
| Bostunib | 0.09135798 | 0.3168 | 23.5 | M2 |
| Su11274 | 0.72193028 | 0.9476 | 17.8 | M2 |
| Alsterpaullone | 0.3076402 | 0.4391 | 13.9 | M2 |
| ALRESTATIN | 3.66821661 | 0.8352 | 15.05 | M2 |

### Example 4: Reprogramming Screen of Compounds on Polarized Macrophages

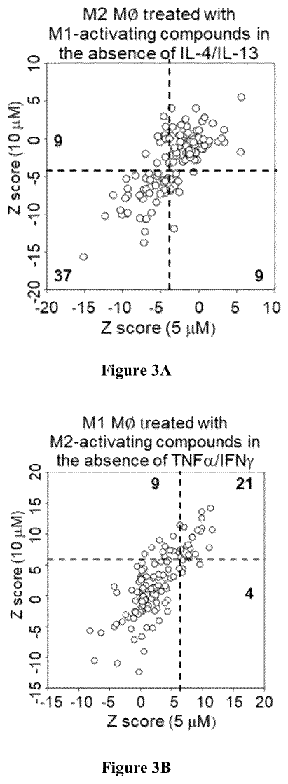

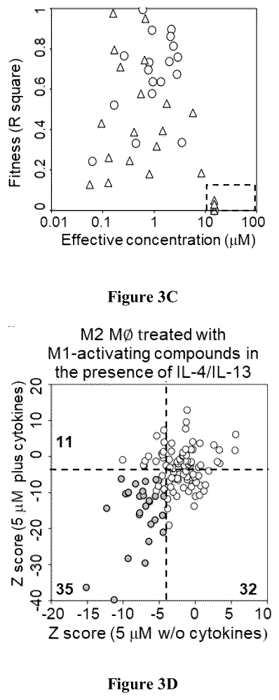

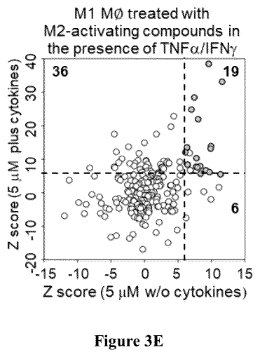

To investigate whether the identified compounds could reprogram or reactivate macrophages after M1- or M2-like differentiation, we rescreened the hits on M1- or M2-activated macrophages. hMDMs were activated into M2-like macrophages by IL-4 plus IL-13 or M1-like macrophages by IFNγ plus TNFα. After removing the differentiating cytokines, M2-like macrophages were treated with each of the 166 M1-activating compounds and M1-like macrophages were treated with each of the 180 M2-activating compounds at a final concentration of either 5 μM and 10 μM. 24 hours later, cell images were taken and cell shapes were quantified. Based on the same Z-score cutoff, 37 M1-activating and 21 M2-activating compounds were identified to induce cell shape changes at the concentration of both 5 μM and 10 μM (FIGS. 3A-3B). Dosage responses were carried out with 40 commercial available compounds (21 M1-activating and 19 M2-activating) on polarized macrophages. 17 of the M1-activating (81%) and 18 of the M2-activating (95%) compounds had typical dosage dependent response with an EC below 10 μM, and induced statistical significant changes of cell shape (FIG. 3C and Table 3).

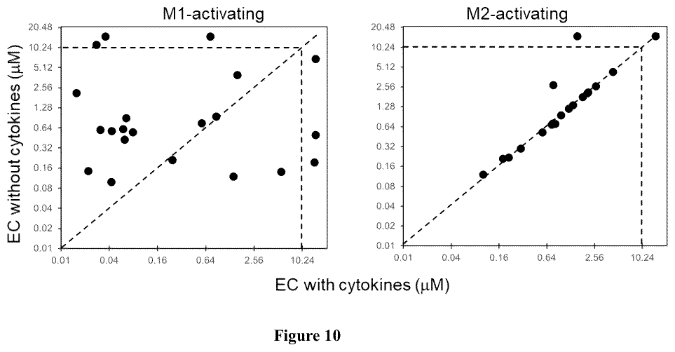

We also rescreened the hits on differentiated macrophages in the presence of differentiating cytokines: either IL-4 plus IL-13 or IFNγ plus TNFα. Surprisingly, more compounds exhibited significant effects on cell shape changes in the presence of these cytokines (67 M1- and 55 M2-activating) than in absence of these cytokines (46 M1- and 25 M2-activating) at the same compound concentration of 5 μM (FIGS. 3D-3E). Consistently, 28 of the 37 M1-activating and 18 of the 21 M2-activating compounds were identified again to induce significant cell shape change at the concentration of 5 μM. In the dosage response assay, the ECs of many M1-activating compounds were lower in the presence of cytokines than in the absence of cytokines (FIG. 10). Thus, the presence of differentiating cytokines makes macrophages more sensitive to reprogramming.

Table 3 shows dosage information of selected compounds on differentiated macrophages

| Compounds | EC | R-square | Category |
|---|---|---|---|
| Bisantrene | 1.984795322 | 0.737 | M2 |
| dihydrochloride | | | |
| triptolide | 0.061894009 | 0.2428 | M2 |
| lovastatin | 0.442115573 | 0.3319 | M2 |
| QS 11 | 0.261082221 | 0.7666 | M2 |
| Regorafenib | 2.882594235 | 0.7596 | M2 |
| Sorafenib | 0.794071491 | 0.7332 | M2 |
| MLN2238 | 3.461032864 | 0.3362 | M2 |
| GW-843682X | 0.897347267 | 0.5783 | M2 |
| KW 2449 | 2.16025641 | 0.8979 | M2 |
| Axitinib | 0.591495199 | 0.9957 | M2 |
| JTE 013 | 0.938113208 | 0.6378 | M2 |
| Purmorphamine | 2.345142857 | 0.8143 | M2 |
| Arcyriaflavin A | 1.342190889 | 0.6374 | M2 |
| Dasatinib | 0.761950413 | 0.6968 | M2 |
| NVP-LDE225 | 2.76599809 | 0.6752 | M2 |
| 1-Naphthyl PP1 | 2.072231834 | 0.8623 | M2 |
| SELAMECTIN | 15 | 0 | M2 |
| MGCD-265 | 0.911173577 | 0.8933 | M2 |
| Bosutinib | 0.164371173 | 0.523 | M2 |
| Cantharidin | 0.158610234 | 0.9764 | M1 |
| Cucurbitacin I | 1.759105431 | 0.53 | M1 |
| Alprostadil | 0.056193353 | 0.1288 | M1 |
| HMN-214 | 1.482432432 | 0.3942 | M1 |
| WP1130 | 15 | 0.05 | M1 |
| MS275 | 0.81097561 | 0.181 | M1 |
| SMER3 | 0.093980962 | 0.4302 | M1 |
| SCH 79797 | 0.219379028 | 0.7105 | M1 |
| dihydrochloride | | | |
| NVP 231 | 5.744680851 | 0.4822 | M1 |
| Prulifloxacin | 15 | 0 | M1 |
| FTY720 | 0.131265421 | 0.262 | M1 |
| DIHYDROCELASTRYL | 15 | 0.03 | M1 |
| DIACETATE | | | |
| Diphenyleneiodonium | 0.336300175 | 0.2451 | M1 |
| chloride | | | |
| Penfluridol | 0.169426434 | 0.7956 | M1 |
| thiostrepton | 0.407871889 | 0.3882 | M1 |
| Evodiamine | 0.679884726 | 0.949 | M1 |
| MITOXANTRONE | 0.559348161 | 0.5764 | M1 |
| HYDROCHLORIDE | | | |
| Quinolinium | 8.365292011 | 0.1839 | M1 |
| Fenbendazole | 0.649293564 | 0.7447 | M1 |
| Niclosamide | 1.12595217 | 0.3178 | M1 |
| Taxol | 0.128848 | 0.1359 | M1 |

### Example 5: Shared and Unique Effects of Identified Compounds on Macrophage Transcription

To broadly validate the identified compounds on macrophage activation (reprogramming) and to shed light on how the compounds activate macrophages, we selected 17 M1- and 17 M2-activating compounds with ECs below 5 μM and performed transcriptional profiling by RNA-seq. M2-like macrophages induced by IL-4 plus IL-13 were treated with each of the 17 M1-activating compounds at its ECs for 24 hours. Similarly, M1-like macrophages induced by IFNγ plus TNFα were treated with each of the 17 M2-activating compounds at its ECs for 24 hours. Different compounds up-regulated and down-regulated different number of genes (FIG. 4A), and a total of 7247 genes exhibited at least a two-fold change after exposure to at least one compound. Hierarchical clustering of Pearson's correlations of DEGs induced by compounds as well as by IFNγ and IL-4 showed that all 17 M1-activating compounds clustered together with IFNγ and all 17 M2-activating compounds clustered together with IL-4 (FIG. 4). Principal component analysis (PCA) of global transcriptional response showed that M1-like macrophages, M2-like macrophages treated with IFNγ, M1-like macrophages treated with IL-4, and M1-like macrophages treated with M2-activating compounds grouped together, whereas M2-like macrophages and M2-like macrophages treated with M1-activating compounds grouped together (FIG. 11A). Although most compounds as well as IL-4 moderately modulated the global gene expression, GSEA of transcriptional functional modules showed that all M1-activating compounds clustered together and up-regulated typical M1 modules (#7, #8) and the TPP modules (#30, #32) (FIG. 4C). All M2-activating compounds clustered together and down-regulated the typical M1 modules (#7, #8) and the TPP modules (#30, #32). The modules (#26, #27), which are down-regulated by LPS, were also down-regulated by M1-activating compounds but up-regulated by M2-activating compounds. Moreover, expression of typical M1 markers CD80 and CD86 was up-regulated by M1-activating compounds and suppressed by M2-activating compounds while expression of the M2 markers CD206 and CD163 was up-regulated by M2-activating compounds and suppressed by M1-activating compounds (FIG. 11C). These results were further validated at transcriptional level by qPCR and at protein level by flow cytometry (FIGS. 11D-11E).

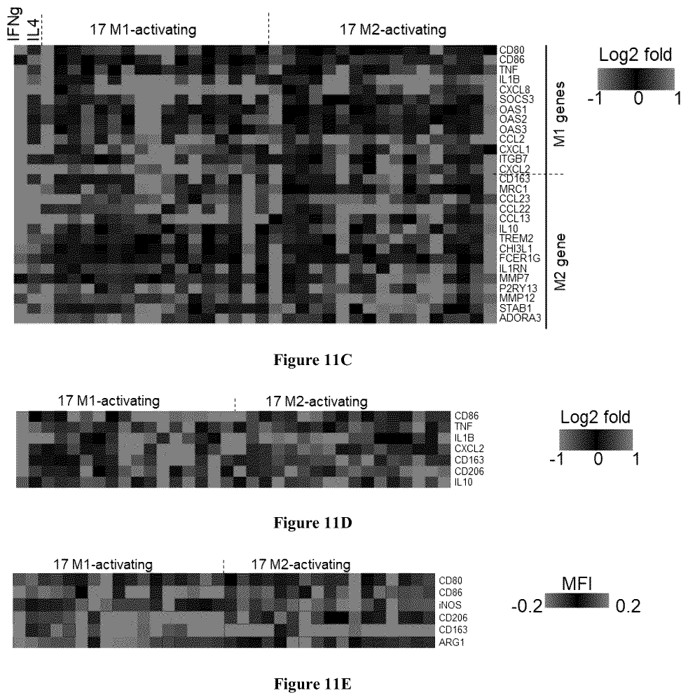

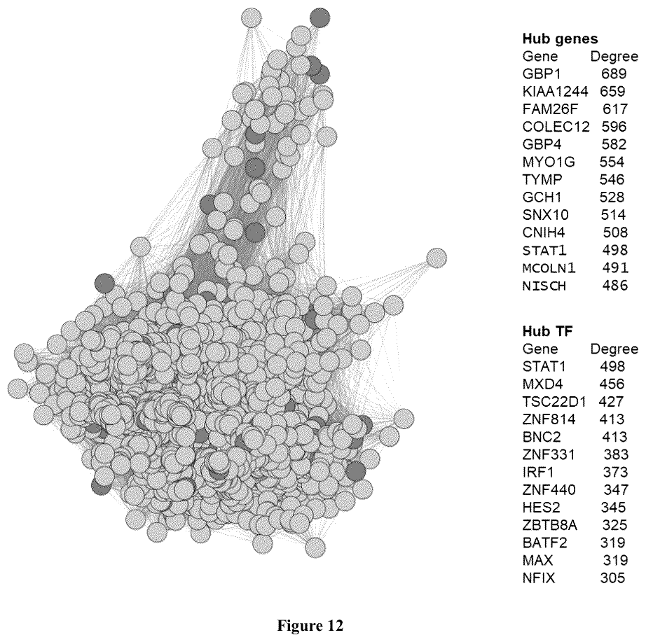

To investigate the common denominators of macrophage activation, a reverse engineering regulatory network was assembled by ARACNe based on mutual information between each gene pair computed from the compound-perturbing expression profiles. Top 10% central hub genes inferred from the network (n=1255 most interconnected genes) collectively participated in 98,048 interactions. Most of top central hub genes or regulators, such as GBP1, FAM26F, STAT1, have been shown to play essential roles in macrophage activation and function (FIG. 12). We performed GO enrichment analysis of these hub genes with visualization of GO enrichment networks by BiNGO. This GO-term network identified functional clusters associated with macrophage activation, including not only previously identified clusters of immune response, leukocyte or lymphocyte activation, catabolic and metabolic process, but also new clusters of stress response, cell migration, protein transport, secretion, cell proliferation, ion homeostasis, phosphorylation and signaling, as well as tissue remodeling and wound healing (FIG. 4D). Moreover, function enrichment analysis of DEGs showed that different compounds not only modulated gene expression in the common immune response pathways and chemotaxis/chemokine-mediated signaling pathway but perturbed specific (unique) pathways (FIGS. 4E-4F and 11B). Consistently, these unique pathways perturbed by compounds were primarily through their putative targets. For example, M1-activating compound MS275 inhibits HDACs (histone deacetylase), which perturbed the pathway of chromatin assembly. M2-activating compound bisantrene inhibits TOP2A (topoisomerase II), which perturbed the pathway of DNA topological change (FIG. 4F). These data suggest that the identified compounds reprogram the differentiated macrophages through modulating the expression of genes associated with macrophage activation as well as specific pathways unique to each compound.

### Example 6: Induction of Macrophages to Proinflammatory State by Thiostrepton

To determine if the identified compounds activate macrophages in disease setting in vivo, we selected thiostrepton, a natural cyclic oligopeptide and an approved veterinary antibiotic for treating skin infection, and tested it to activate macrophages to M1-like state. Similar to other thiopeptide antibiotics, thiostrepton inhibits the ribosome function of bacterial protein synthesis. Recently, thiostrepton was shown to exhibit antiproliferative activity in human cancer cells through inhibiting proteasome and/or FOXM1 transcription factor. Following treatment of hMDMs with 2.5 μM thiostrepton for 24 hours, hMDMs were polarized to express proinflammatory cytokines TNFα and IL-1β and down-regulate the M2 chemokine CCL24 (FIG. 5A). Functional enrichment analysis of the DEGs showed that IFN/NFκB pathway, TNF-mediated pathway, oxidative-reduction process, protein polyubiquitination and cellular response to LPS were upregulated, while DNA replication, cell cycle and cell matrix adhesion were down-regulated (FIG. 5B). GSEA analysis showed pathways of TNFα signaling via NFκB and ROS were upregulated while pathways of E2F target and mitotic spindle were down-regulated (FIG. 5C). These results show that thiostrepton regulates the expression of genes associated with proteasome and DNA replication in hMDMs.

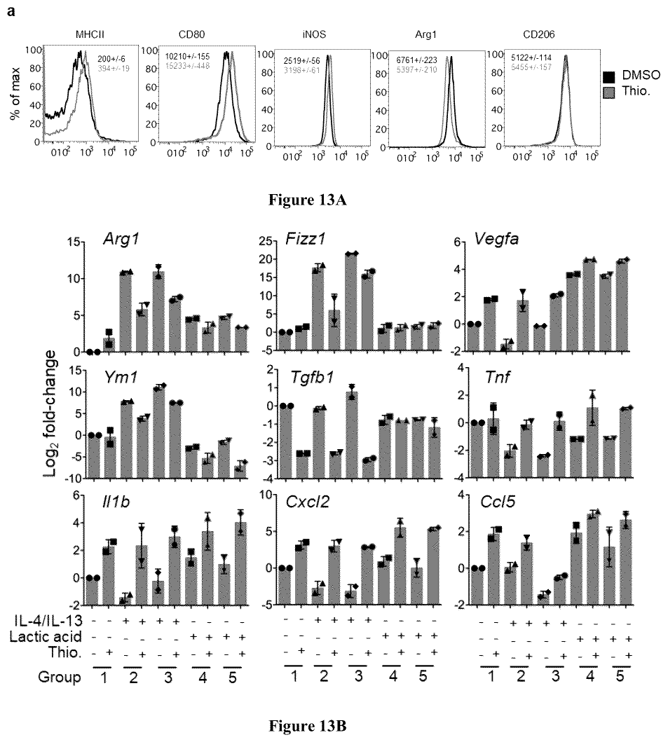

To determine the effect of thiostrepton on TAM in vitro, mouse bone marrow macrophages (BMMs) were cultured in the conditioned medium (CM) of B16F10 tumor cells in the absence or presence of thiostrepton for 24 hrs. Alternatively, BMMs were cultured in the conditioned medium for 24 hrs first and then treated with thiostrepton for another 24 hrs. The expression of selected genes associated with macrophage polarization was assayed by qPCR. Thiostrepton inhibited the expression of TAM/M2-associated genes Arg1, Fizz1, Vegfa, Ym1 and Tgfb but up-regulated the expression of M1-associated genes Tnf, Il1b, Cxcl2 and Nos2 (FIG. 5D). The effect of thiostrepton was observed whether thiostrepton was added into the conditioned medium culture or BMMs were differentiated into TAM first (Compare groups 2 and 3 in FIG. 5D). Consistently, flow cytometry analysis revealed up-regulation of MHCII, CD80 and iNOS but down-regulation of ARG1 (FIG. 13A). Similarly, we examined the effect of thiostrepton on IL-4/IL-13 and lactic acid-polarized BMMs. As shown in FIG. 13B, thiostrepton inhibited the expression of Arg1, Fizz1, Ym1 and Tgfb but elevated expression of Tnf, Il1b, Cxcl2 and Ccl5 whether thiostrepton was added together with cytokines or lactic acid or after BMM polarization.

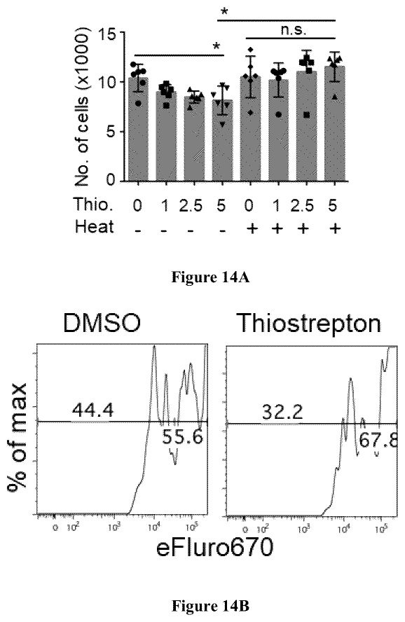

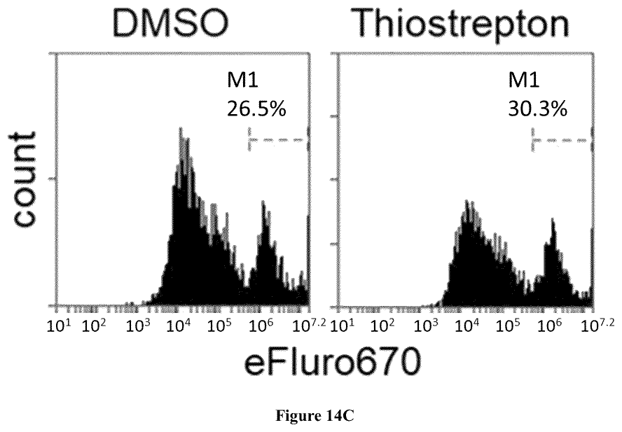

To examine whether thiostrepton-activating macrophages or conditioned medium have effects on tumor cell growth, BMMs were treated with thiostrepton for 24 hrs. Equal numbers of primed BMMs and melanoma cells (B16F10) were co-cultured for 12 hrs. Significantly more melanoma cells were lost in the presence of thiostrepton-treated macrophages as compared to the untreated macrophages in a dose-dependent manner (FIG. 5E). Similarly, more melanoma cells were lost in the conditioned medium from thiostrepton-treated macrophages than conditioned medium from untreated macrophages or heat-inactivated thiostrepton-treated conditioned medium (FIG. 14A). To determine whether thiostreption-activated macrophages exhibit enhanced ADCP, thiostreption-activated macrophages were co-cultured with equal number of human B lymphoma cells (GMB) labeled with eFluro670 dye and anti-CD20 for 2 hrs. Thiostrepton elevated ADCP of both human and mouse macrophages (FIGS. 14B-14C). These data show that thiostrepton activates and reprograms macrophages toward a pro-inflammatory state and enhances their tumor-killing activity in vitro.

### Example 7: Reprogramming TAMs for Enhanced Anti-Tumor Activity In Vivo by Thiostrepton

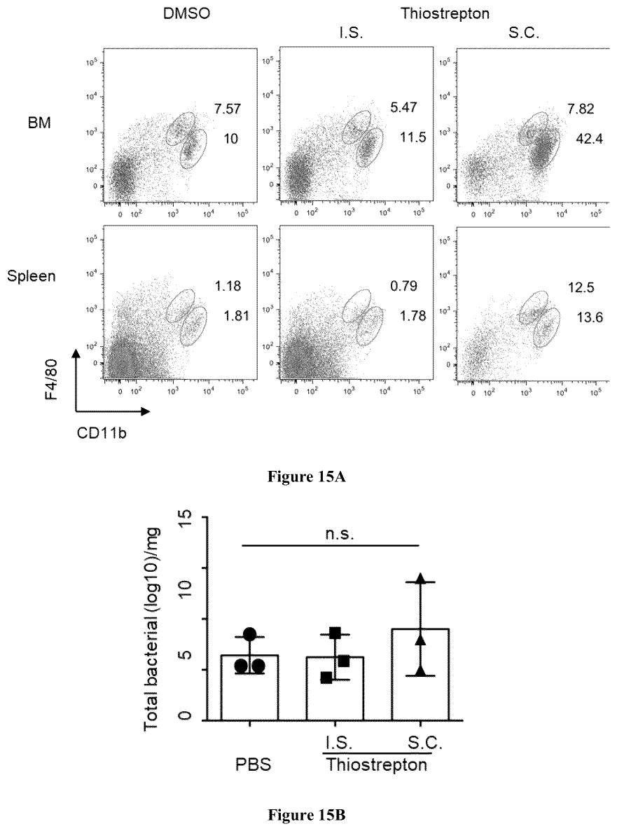

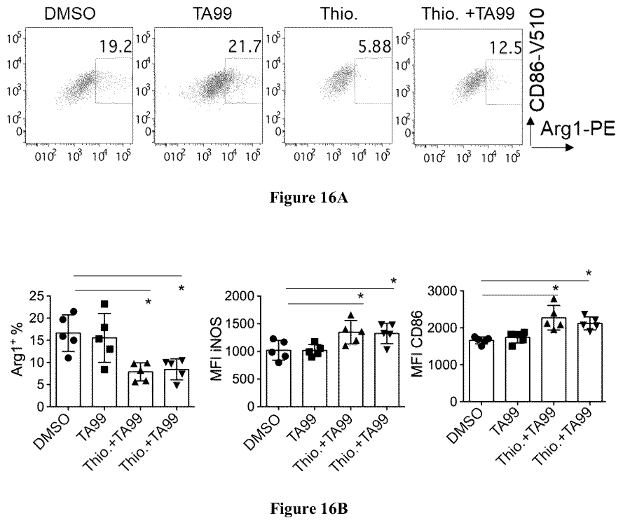

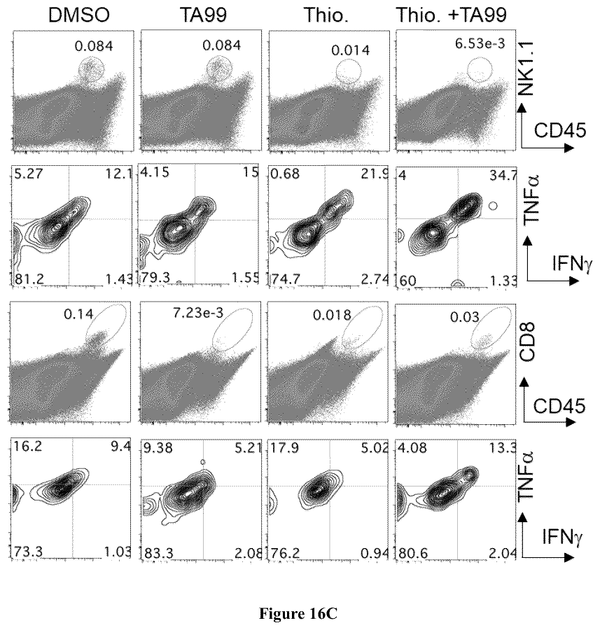

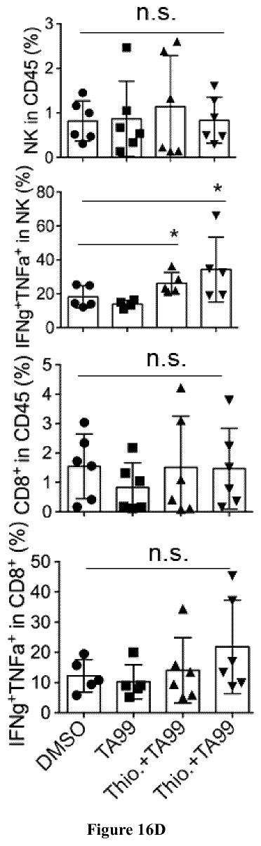

Next, we examined whether thiostrepon has anti-tumor effect in vivo through activating macrophages. B16F10 melanoma cells were injected subcutaneously into syngeneic C57BL/6 mice. 6 and 12 days later, tumor-bearing mice were treated with either vehicle (DMSO), melanoma specific antibody TA99, thiostrepton, or combination of TA99 and thiostrepton by intraperitoneal injection (I.P.). In a dosage-dependent manner (150 or 300 mg/kg), thiostrepton strongly suppressed the tumor growth alone and additively with TA99 (FIG. 6A). Since thiostrepton inhibits cell proliferation and is an antibiotic, to exclude its systematic effects on immune cells and on gut microbiome, tumor-bearing mice were treated by para-tumor subcutaneous injection (S.C.) with a lower dose of thiostrepton (20 mg/kg). This local treatment also suppressed the tumor growth and exhibited additive effects with TA99 (FIG. 6B). Flow cytometry analysis of single cell suspensions of dissected tumors at day 18 post tumor engraftment showed elevated levels of macrophages and monocytes in mice given thiostrepton or thiostrepton plus TA99 as compared to mice given vehicle or TA99 (FIGS. 6C-6D). Consistently, more abundant macrophages were stained positive for F4/80 by immunochemistry in tumor sections from mice treated with thiostrepton or thiostrepton plus TA99 than mice treated with vehicle or T99 (FIG. 6E). In non-tumor bearing mice, intraperitoneal administration of thiopstrepton led to increased numbers of macrophages in the spleen and bone marrow while subcutaneous administration did not have significant effects on macrophage numbers (FIG. 15A). In both dosing strategies, thiostrepton did not change the total bacterial counts in the gut (FIG. 15B). Moreover, flow cytometry analysis of TAM revealed elevated levels of iNOS and CD86 and decreased levels of Arg1 in mice given thiostrepton or thiostrepton plus TA99 as compared to mice given vehicle or TA99 (FIGS. 16A-16B). Interestingly, an increased number of TNFα⁺ IFNγ⁺ NK cells (but not CD8⁺ T cells) was found in tumors in mice given thiostrepton or thiostrepton plus TA99 as compared to mice given vehicle or TA99 (FIG. 16C).

To investigate whether tumor-infiltrated macrophages were reprogrammed, we purified TAMs from B16F10 melanoma tumors from mice dosed with thiostrepton or vehicle by I.P. or S.C. at day 18 post tumor engraftment and performed RNA-seq. GSEA and functional enrichment analysis showed that thiostrepton up-regulated the expression of genes associated with inflammatory response and ROS and down-regulated the expression of genes associated with mitotic division in TAMs from mice treated with thiostrepton by both I.P. and S.C. (FIG. 17). The expression of the pro-inflammatory cytokines, including Tnf, Il1b, Cxcl1 and Cxcl2, were also significantly upregulated (FIG. 6F), consistent with the results from thiostrepton treatment of hMDMs in vitro (FIG. 5A).

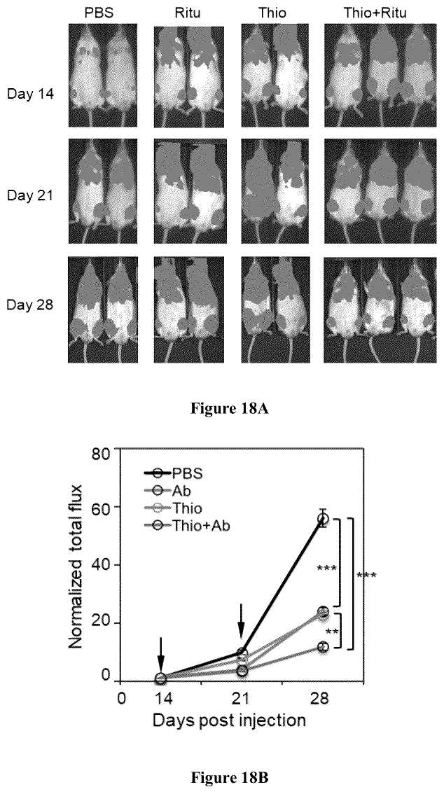

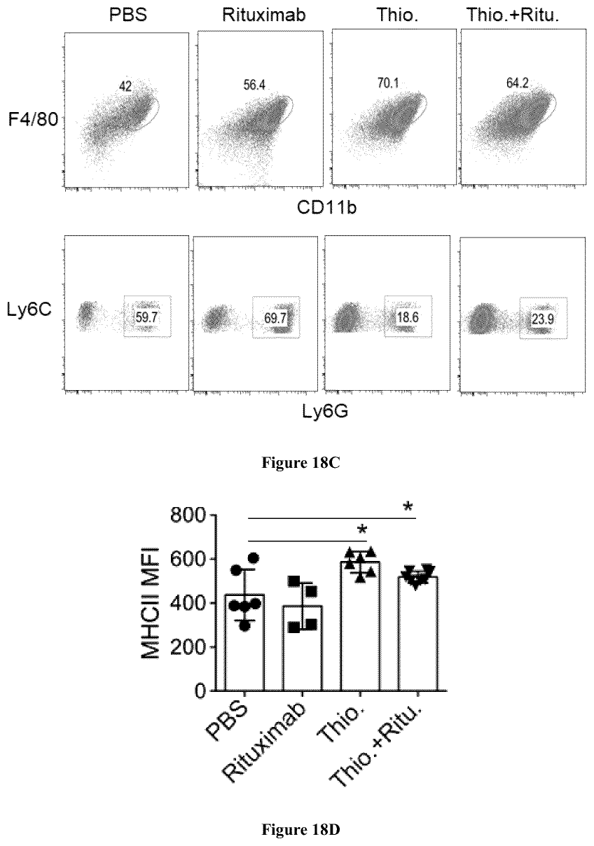

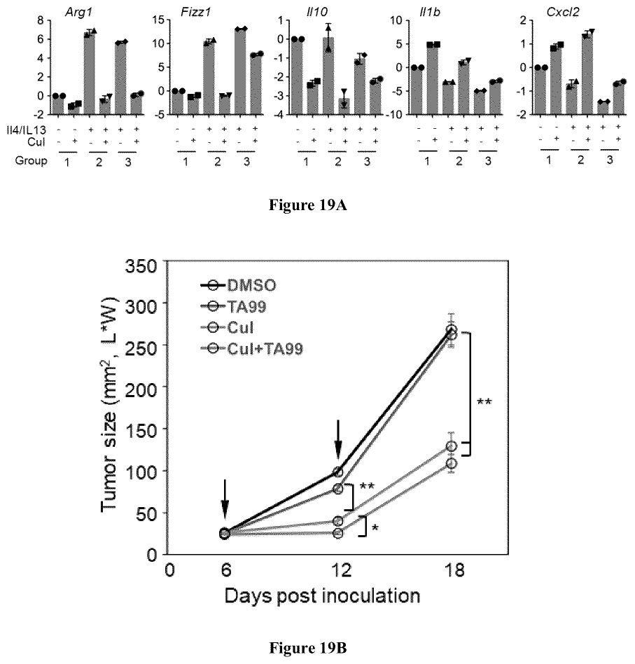

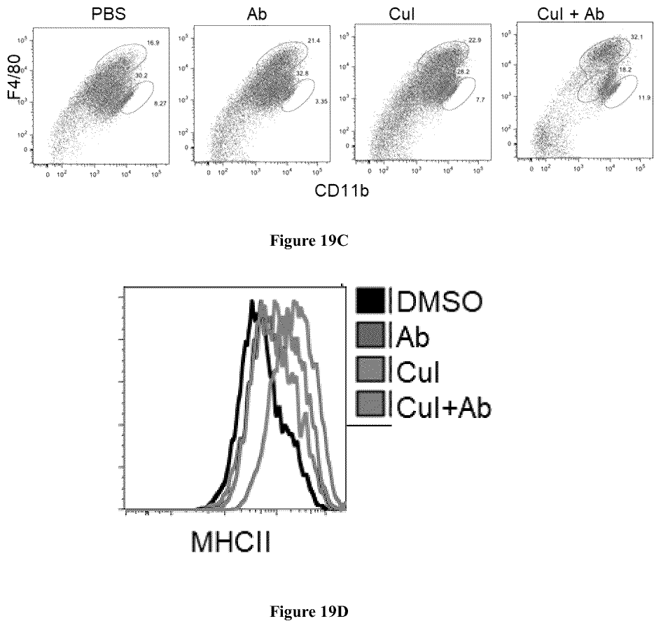

To further confirm the anti-tumor effects of thiostrepton in vivo, we injected i.v. luciferase-expressing human B lymphoma cells into NSG mice. Tumor-bearing mice were treated with rituximab (anti-CD20), thiostrepton or both at 2 and 3 weeks post tumor engraftment. Quantification of tumor burden by luciferase imaging showed that thiostrepton alone or together with rituximab significantly reduced the tumor burden in the bone marrow (FIGS. 18A-18B). Consistently, higher percentages of F4/80⁺CD11b⁺ macrophages with higher expression of MHCII were found in the bone marrow of mice treated with thiostrepton than mice given vehicle or rituximab whereas the frequencies of Ly6G⁺ neutrophils were lower (FIGS. 18C-18D). Moreover, another M1-activating compound, cucurbitacin I, also inhibited B16F10 growth by activating macrophages both in vitro and in vivo (FIG. 19). Taken together, M1-activating compounds could reprogram TAMs into pro-inflammatory macrophages to inhibit tumor growth in vivo.

### Example 8: Material and Methods

### Mice, Antibodies, Cell Lines and Plasmids

C57BL/6 (B6) mice, p47phox^{-/-}, Clec4f-Cre mice were purchased from the Jackson Laboratory and maintained in the animal facility at the Massachusetts Institute of Technology (MIT). PKM^{flox} mice were described in the previous publication. Antibodies specific for CD11b (M1/70), F4/80 (BM8), MHC-II (M5/114.15.2), CD45.2 (104), CD9 (MZ3) for flow cytometry were from Biolegend. Anti-GPR3 (#SC390276) was from Santa Cruz Biotechnology. Anti-β-arrestin2 (#4674), Glycolysis Antibody Sampler Kit (#8337), anti-Myc and anti-FLAG were from Cell Signaling Technology. Anti-PKM2 (#1C11C7) was from Abcam. β-Arrestin2 CRISPR plasmids (sc432139) was from Santa Cruz Biotechnology. pCMV-β-arrestin2-GFP (PS10010), pCMV6-Flag-myc-barrestin2 (PS100001) and Arrb2 mouse siRNA Oligo Duplex (Locus ID 216869) were from Origene. Immortalized Kupffer cell line (ABI-TC192D, AcceGen), human primary KCs (ABC-TC3646, AcceGen), THP-1 (ATCC TIB-202) and 293T (CRL-3216) were cultured following vendor instructions (37° C., 5% CO₂). Transfection of ImKCs with siRNAs was accomplished using Lipofectamine™ 2000 (Thermo Fisher Scientific) according to the manufacturer's instruction. Apocynin (PHL83252) was from Sigma.

### Bone Marrow Derived Macrophages (BMDMs)

Mouse BMDMs were prepared. Fresh bone marrow cells were isolated from B6 mice, plated onto a six-well plate with 1×10⁶/mL in complete RPMI with 2-mercaptoethanol and 20% L929 supernatants which were obtained by culturing L-929 cells for 6 days with medium change every 2 days.

### Co-Immunoprecipitation, Western Blotting and Native PAGE

293T cells were transfected with FLAG-tagged β-arrestin2, using TransIT®-LT1 Transfection Reagent (Mirus). Thirty-six hours after transfection, the cells were lysed using cold Lysis Buffer containing 20 mM Tris-HCl (pH 7.4), 150 mM NaCl, 0.1% NP-40, 10% glycerol, proteinase inhibitor (Roche Catalog No. 11836153001), and phosphatase inhibitors (Roche Catalog No. 04906845001). The clear supernatants from the lysate were incubated with M2-magnetic beads conjugated with anti-FLAG antibody (Sigma Catalog No. M8823) for 2 hours at 4° C. Then the beads were washed twice and eluted by the 3×FLAG peptides (Sigma Catalog No. F4799) as described in the Sigma manual for Western blotting.

Proteins were extracted from cells with RIPA buffer. Protein concentration was quantified by BCA Protein Assay Kit (Pierce Biotechnology). Samples containing 20 μg total protein were resolved on a 10% SDS-PAGE gel and electro-transferred onto a PVDF membrane (Millipore Corporation). The membrane was blocked in 5% (w/v) fat-free milk in PBST (PBS containing 0.1% Tween-20). The blot was hybridized overnight with primary antibodies: anti-pSRC (D49G4, Cell Signaling Technology, 1:1000) and pSIK1/2/3 (#ab199474, Abcam, 1:1000) according to the recommended dilution in 5% fat-free milk. The blot was washed twice in PBST and then incubated with anti-Rabbit HRP-conjugated secondary antibody (Cell Signaling Technology, 1:2000) in 5% fat-free milk. The membrane was washed twice in PBST and subjected to protein detection by ECL Plus Western Blotting Detection System (GE Healthcare) before being exposed to a Kodak BioMax XAR film. The membrane was stripped and reblotted with the anti-β-tubulin (D49G4, Cell Signaling Technology) for protein loading control.

Protein was extracted from cells in 1× Native PAGE sample buffer (ThermoFisher) containing 1% digitonin followed by 20 min spin at 12,000×g to pellet debris. Protein extracts were analyzed using NativePAGE Novex System (ThermoFisher) and subsequently transferred to PVDF membrane, fixed, and blotted for native proteins.

### Metabolite Profiling

ImKCs were treated with DPI (#81050, Cayman) at 50 or 500 nM for 6 hrs or 24 hrs. Cells were washed once in ice-cold 0.9% NaCl and lysates were extracted in 80% methanol solution containing internal standards for LC/MS by scraping on dry ice followed by 10-minute mixing with vortex in 4° C. Following lysate extraction, debris were removed by high-speed centrifugation and supernatant was dried using speedvac. Samples were analyzed by LC/MS on QExactive Orbitrap instruments (Thermo Scientific) in Whitehead Institute metabolite profiling core facility. Data analysis was performed using the in-house software described previously (Lewis et al., 2014).

β-arrestin2 Nuclear Translocation Assay

BMDMs or ImKCs were cotransfected with plasmids encoding FLAG-GPR3-GFP or β-arrestin2-RFP. Twenty-four hours after transfection, cells were reseeded into a 24-well glass-bottom plate (Nest, Shanghai, China) and treated with DPI (50 nM), S1P (3 mM), or vehicle control (0.3% DMSO) for the indicated duration. The fluorescent signals of membrane-bound receptor or β-arrestin2 were collected as live images using a total internal reflection fluorescence (TIRF) microscope (Olympus).

### Oxygen Consumption, Glucose Stress Assay, Glucose Consumption and Lactate Production

OCR and ECAR were measured in isolated tissues or cultured ImKCs using the Seahorse XFe Extracellular Flux Analyzer (Agilent). For tissue respiration assays, 1.0 mg adipose tissue was dissected from inguinal WAT depots by using a surgical biopsy instrument (Integra Miltex Standard Biopsy Punches, Thermo Fisher) and placed into XF96 Islet Capture Microplates and pre-incubated with XF assay medium with pH value at 7.4. XF assay medium supplemented with 1 mM sodium pyruvate, 2 mM GlutaMax™-I, and 25 mM glucose. Isolated MDMs or Kupffer cells were subjected to a mitochondrial stress test by adding oligomycin (2 μM) followed by carbonyl cyanide 4-(trifluoromethoxy), phenylhydrazone (FCCP, 5 μM), and antimycin (1 μM). For glucose stress assay and ECAR measurement, XF assay medium was supplemented only with GlutaMax™-I. Tissue or cells were subjected to a glucose stress test by adding highly concentrated glucose (for tissue, 25 mM; for cells, 10 mM), followed by adding oligomycin (5 μM), FCCP (5 μM), and 2-DG (50 mM). Cells were seeded in culture dishes, and the medium was changed after 6 hours with serum-free DMEM. Cells were incubated for 12-16 hours, and the culture medium was then collected for measurement of glucose and lactate concentrations. Glucose levels were determined using a glucose (GO) assay kit (Sigma). Glucose consumption was the difference in glucose concentration when compared with DMEM. Lactate levels were determined using a lactate assay kit (Eton Bioscience).

### Immunofluorescence and Microscope

BMDMs or Kupffer Cells were fixed and incubated with primary antibodies, and then labeled with Alexa Fluor dye-conjugated secondary antibodies and counterstained with Hoechst 33342 according to standard protocols. Cells were examined using a deconvolution microscope (Zeiss) with a 63-A oil immersion objective. Axio Vision software from Zeiss was used to deconvolute Z-series images.

### PKM and GAPDH Enzymatic Activity

The enzymatic activities of PKM and GAPDH were measured using the pyruvate kinase activity assay kit (Biovision, #K709) and GAPDH activity assay kit (Biovision, #K680) according to the manufacturer's protocols, respectively.

### Myc Luciferase Assay

The c-Myc activity was assessed using the Myc Reporter kit (BPS Biosciences) and the Dual-Luciferase Reporter System (Promega) according to the manufacturers' instructions. Briefly, 100 μL (1.5×10⁵ cells/mL) control and Kupffer cells were seeded into 96-well plates. After overnight incubation, when cells reached ˜50% confluency, 1 μL of Reporter A (60 ng/μL) in the Myc Reporter kit was transfected into cells using Turbofectin 8.0. After 48 hours, cells were lysed in 25 μL Passive Lysis Buffer (provided in the Dual-Luciferase Reporter kit). 20 μL of cell lysate was transferred to 96-well plates and placed in a 96-well microplate luminometer (GloMax-Multi, Promega). 100 μL Luciferase Assay Reagent II and 100 μL Stop & Glo Reagent (both provided in the Dual-Luciferase Reporter kit) were sequentially injected, and firefly and Renilla luciferase activities were automatically measured. c-Myc activities were determined by the ratios of firefly to Renilla luciferase activities.

### HFD-Induced NAFLD Mouse Model and Treatment

C57BL/6 mice at 5 weeks of age (body weight=23-25 g) were randomly assigned to three groups: 5 mice were fed with a normal chow diet for 16 weeks and then injected with saline once every 5 days for 4 weeks; 10 mice were fed with HFD (60 kcal % fat) for 16 weeks to induce obesity and hepatosteatosis and then divided into two groups: HFD+vehicle (HFD) group (n=5) was injected with the vehicle (PEG3000) and HFD+DPI group (n=5) was injected with DPI in vehicle (2 mg/kg) i.p. every 5 days for 4 weeks.

### Histopathology and Immunochemical Staining

Liver samples fixed in 10% buffered formalin were embedded in paraffin, sliced (2 μm sections), and stained with hematoxylin and eosin (H&E). Histological examination for morphological changes was performed in a blinded manner. Liver sections were scored according to the criteria of the NAFLD activity score (NAS).

### Glucose Tolerance Test (GTT)

The GTT were performed in mice 19 weeks after feeding with HFD or NC. For GTT, mice were fasted overnight, followed by an intraperitoneal injection of 1 g/kg glucose. For the ITT, mice were fasted for 6 hours, followed by an intraperitoneal injection of 0.75 units/kg insulin. Blood was obtained from the tail vein before (0 min) and after (15, 30, 60, 90 and 120 min) the injection of glucose or insulin. Glucose levels were measured using an automatic glucometer (Roche Diagnostics, Rotkreuz, Switzerland).

### Human Liver Immune Cell Isolation and Kupffer Cell Isolation

Human liver biopsies were obtained from livers procured from deceased donors deemed unacceptable for liver transplantation. Samples were collected with appropriate institutional ethics approval from The First Affiliated Hospital of Jilin University. All experiments were performed in accordance with the relevant guidelines and regulations. In addition, written informed consent was obtained from each subject. During organ retrieval, donor liver grafts were perfused in situ with cold (HTK) solution (Methapharm) to thoroughly flush out circulating cells, leaving only tissue resident cells that are then used to prepare a single-cell suspension to isolate immune cells. The unused liver caudate lobe post liver transplantation was collected and flushed with HBS+EGTA at 4° C. to remove any non-liver resident cells. Single-cell isolation from the resected caudate lobe was performed with a modified two-step collagenase procedure (MacFarland et al. 2017 ACnano). Single cell suspension was stained with anti-CD45 to sort all immune cells for scRNAseq or anti-CD14 to sort KCs for in vitro treatment by flow cytometry (BD Aria).

### RNA Isolation, Sequencing, and Data Analysis

Mouse livers were dissected and digested with Collagenase IV (Roche). Single cell suspension was stained with anti-F4/80, anti-CD11b and anti-Gr-1. F4/80⁺CD11b⁺Gr1^{low} macrophages were sorted by flow cytometry (BD Aria). RNAs were extracted with RNeasy MinElute Kit (Qiagen), converted into cDNA and sequenced using Next-Generation Sequencing (Illumina). RNA-seq data were aligned to the human genome (version hg19) and raw counts of each genes of each sample were calculated with bowtie2 2.2.3 (Langmead et al. 2009) and RSEM 1.2.15 (Li et al. 2011). Differential expression analysis was performed using the program edgeR at P<0.05 with a two-fold change (Robinson et al. 2010). The gene expression level across different samples was normalized and quantified using the function of cpm. DEGs were annotated using online functional enrichment analysis tool DAVID (Huang et al. 2007).

### Single Cell RNAseq and Computational Analysis

Sorted CD45⁺ cells were resuspended and washed in 0.05% RNase-free BSA in PBS for single-cell library preparation with 10× Chromium Next GEM Single Cell 3′ Kit (10×Genomics according to the manufacturer's instructions. The single-cell cDNA libraries were sequenced by NexSeq500 (Illumina). Raw sequences were demultiplexed, aligned, filtered, barcode counting, unique molecular identifier (UMI) counting with Cell Ranger software v3.1 (10×Genomics) to digitalize the expression of each gene for each cell. The analysis was performed using the Seurat 3.0 package. We first processed each individual data set separately prior to combining data from multiple samples. The outlier cells with extreme low number (<500) or high number (>5,000) of gene features as doublets, or low total UMI (<1,000) and high mitochondrial ratio (>15%) from each data set were removed. Subsequently, samples were combined based on the identified anchors for the following integrated analysis. We ran principal component analysis (PCA) and used the first 15 principal components (PCs) to perform tSNE clustering. We checked well-defined marker genes for each cluster to identify potential cell populations, such as T cells (CD3E, CD8A, CD4, CD69, IL7R), B and plasma cells (CD19, MS4A1, SDC1), DC (CD11C, CLEC9A), NK cells (CD56, CD16, GZMB). For macrophage analysis, CD14 and CD68 positive clusters were selected for subsequent analyses. We repeated PCA, tSNE clustering on the integrated data sets of macrophages. Differential expression analysis was performed to identify the genes significantly upregulated in each cluster compared with all other cells. For gene sets representing specific cellular functions or pathways, we performed functional enrichment analysis with the biological process of Gene Ontology by the online tool DAVID.

### Statistic Methods

Statistical significance was determined with the two-sided unpaired or paired Student's t test. The FDRs were computed with q=P×n/i, where P=P value, n=total number of tests, and i=sorted rank of P value.

### Example 9: DPI Stimulates Both Rapid and Sustained Increase in Glycolysis in Macrophages

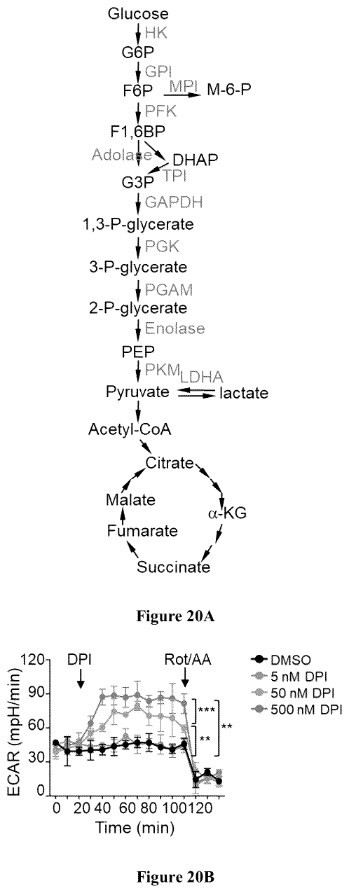

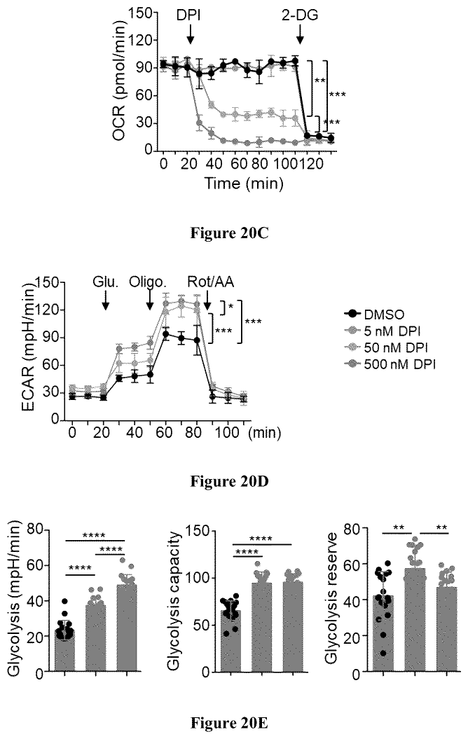

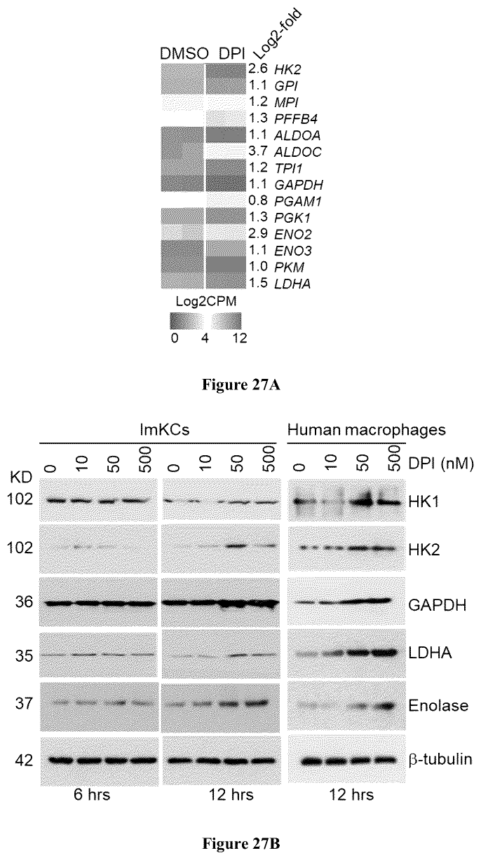

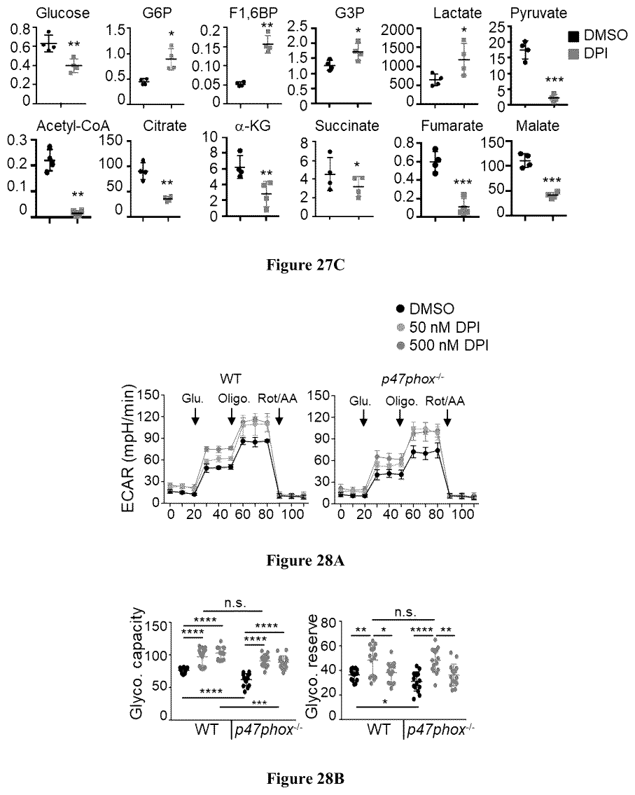

DPI stimulates transcription of many genes in the glycolysis pathway in human primary macrophages (FIG. 20A and FIG. 27A). We confirmed the upregulation of hexokinase (HK), glyceraldehyde-3-phosphate dehydrogenase (GAPDH), lactate dehydrogenase A (LDHA) and enolase at the protein level in both human primary macrophages and an immortalized line of mouse Kupffer cells (ImKCs) in an DPI dose- and treatment time-dependent manner (FIG. 27B). To investigate the effect of DPI on cellular metabolism, we measured cellular activities in glycolysis and oxidative phosphorylation (OxPhos) by assaying extracellular acidification rate (ECAR) and oxygen consumption rate (OCR), respectively, in ImKCs in the absence or the presence of 5, 50 and 500 nM DPI. In a dose-dependent manner, DPI stimulated an immediate increase in ECAR and a concomitant decrease in OCR (FIGS. 20B-20C). The DPI-stimulated increase in glycolysis was sensitive to glucose, oligomycin, and rotenone plus antimycin A, and was associated with significant increase in glycolytic capacity and reserve (FIGS. 20D-20E). The effects of DPI on glycolysis and OxPhos were confirmed by quantifying the levels of the major intermediates in the glycolysis pathway and the tricarboxylic acid (TCA) cycle in ImKCs 6 hours after DPI treatment. As shown in FIG. 20F, in a DPI dose-dependent manner, glucose level decreased significantly while the levels of intermediates in the glycolysis pathway, including glucose 6-phosphate (G6P), fructose 1,6-bisphosphare (F1,6BP), glyceraldehyde 3-phosphate (G3P), pyruvate, and lactate increased significantly. In contrast, the levels of TCA cycle intermediates, including acetyl-CoA, citrate, α-ketoglutarate (α-KG), succinate, fumarate and malate, all decreased in a DPI dose-dependent manner. Similar changes in the levels of glucose, glycolysis and TCA cycle intermediates were also seen 24 hours after DPI treatment (FIG. 27C). These results show that DPI regulates cellular metabolism dynamically at two levels: rapid stimulation of glycolysis with concomitant inhibition of OxPhos and sustained stimulation of glycolysis by upregulating transcription and expression of genes in the glycolysis pathway.

### Example 10: DPI Stimulates Glycolysis Through GPR3 and β-Arrestin2

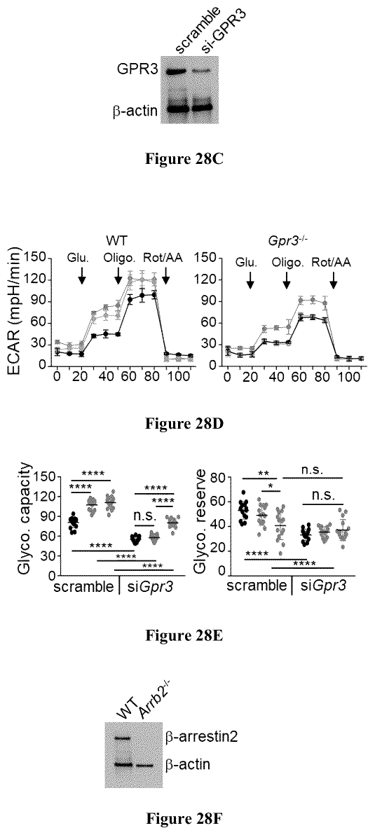

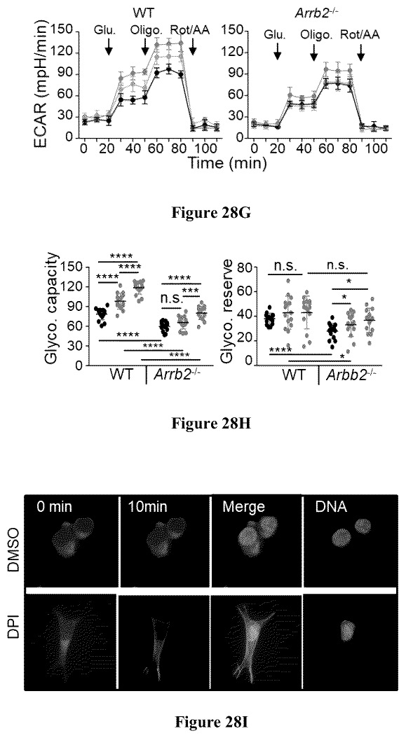

DPI is an agonist of GPR3 and an inhibitor of GAPDH oxidase (NOX). We first determined the requirement of NOX in DPI-stimulated glycolysis. Bone marrow derived macrophages (BMDMs) were prepared from p47phox^{-/-} mice, which do not have any functional NOX activity as p47phox is the organizer of phagocyte NAPDH oxidase (NOX2). Compared to wild-type (WT) BMDMs, p47phox^{-/-} BMDMs had a significantly lower basal level of glycolysis, glycolytic capacity and glycolytic reserve (FIGS. 21A-21C and FIGS. 28A-28B). However, DPI stimulated similar levels of increase in glycolysis, glycolytic capacity and glucose consumption in a dose-dependent manner in both wild-type and p47phox^{-/-} BMDMs. Similarly, DPI stimulated similar increase in glycolysis in ImKCs when NOX activity was pharmacologically inhibited by apocynin, a NOX specific inhibitor (FIG. 21B). These data show that DPI-stimulated glycolysis is independent of NOX activity.

To determine the requirement of GPR3, we knocked down GPR3 by siRNA (siGpr3) in ImKCs. Although GPR3 knockdown was about 70% (FIG. 28C), the basal level of glycolysis and glycolytic capacity were significantly decreased in siGpr3 ImKCs as compared to ImKCs transfected with scramble siRNA (FIG. 21D). Importantly, at 50 nM, DPI did not stimulate any increase in glycolysis, glycolytic capacity and glucose consumption in siGpr3 ImKCs as compared to controls (FIGS. 21D-21E and FIGS. 28D-28E). However, at 500 nM, DPI stimulated a significant increase in glycolysis and glycolytic capacity in siGpr3 ImKCs, but the magnitude of increase was significantly lower than that in scramble siRNA transfected ImKCs, probably due to the partial knockout of GPR3 by siRNA or stimulation of other proteins by DPI. Moreover, sphingosine-1-phosphate (S1P), a reported endogenous ligand of GPR3, also stimulated a significant increase in glycolysis in ImKCs, although the magnitude of increase was much lower than that stimulated by 50 nM DPI (FIG. 21F), showing that activation of GPR3 by an endogenous ligand also stimulates glycolysis in macrophages.

β-arrestin2, encoded by Arrb2, has been reported to bind to GPR3 and is required for GPR3 signaling. To investigate the requirement of 0-arrestin2 in DPI-stimulated glycolysis, we constructed Arrb2^{-/-} ImKCs using CRISPR-Cas9 mediated gene editing (FIG. 28F). The same as siGpr3 ImKCs, the basal level of glycolysis and glycolytic capacity were significantly decreased in Arrb2^{-/-} ImKCs as compared to parental ImKCs (FIGS. 21G-21H and FIGS. 28G-28H), and at 50 nM, DPI did not stimulate any increase in glycolysis and glycolytic capacity in Abbr2^{-/-} ImKCs. Moreover, DPI, but not S1P, stimulated translocation of β-arrestin2 from cytosol to the plasma membrane in 10 min in both ImKCs and BMDMs (FIG. 21I and FIG. 28I).

Together, these results show that DPI-stimulated glycolysis is dependent on GPR3 and β-arrestin2 and that activation of GPR3 by DPI leads to rapid trafficking of 0-arrestin2 to the plasma membrane.

### Example 11: DPI Stimulates Rapid Increase in Glycolytic Activity Through the Formation of GPR3-β-Arrestin2-GAPDH-PKM2 Super Enzymatic Complex

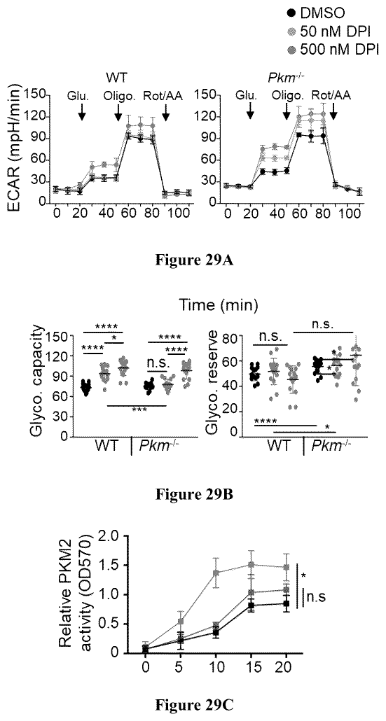

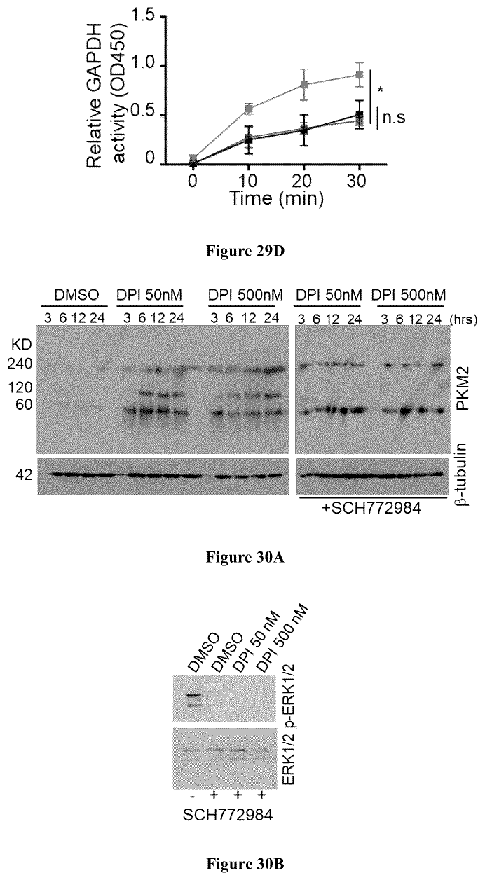

How does DPI stimulate a rapid increase in glycolytic activity? We investigated the interaction between β-arrestin2 and metabolic enzymes, including PKM2 and GAPDH. To investigate this mechanism, we treated ImKCs with or without DPI for 6 hours and immunoprecipitated β-arrestin2 followed by Western blotting analysis. ERK1/2, enolase, GAPDH and PKM2 co-precipitated with β-arrestin2 (FIG. 22A). Notably, significantly higher levels of GAPDH and PKM2 co-precipitated with β-arrestin2 following DPI treatment, suggesting that DPI promotes interactions between β-arrestin2 and GAPDH and PKM2. To determine the requirement of PKM2 in DPI-induced glycolysis, we treated BMDMs from wild-type and Pkm^{-/-} mice with DPI and measured glycolytic activity. The same as siGpr3 ImKCs and Arrb2^{-/-} ImKCs, 50 nM DPI did not stimulate any increase in glycolysis, glycolytic capacity and glucose consumption of Pkm^{-/-} BMDMs (FIGS. 22B-22C and FIGS. 29A-29B). We also measured the enzymatic activity of PKM2 and GAPDH in parental and Arrb2^{-/-} ImKCs in the absence or the presence of 50 nM DPI. As shown in FIGS. 22D-22E, DPI stimulated an immediate increase in PKM2 and GAPDH enzymatic activities in an β-arrestin2-dependent manner. Moreover, DPI's effect on PKM2 and GAPDH enzymatic activities were abolished when phosphorylation of ERK1/2 was inhibited by aapocynin (FIG. 29C). Thus, DPI stimulates the formation of GPR3-β-arrestin2-GAPDH-PKM2 complex, leading to enhanced enzymatic activities of PKM2 and GAPDH, and providing a mechanistic explanation for the observed rapid increase in glycolytic activity following DPI treatment.

### Example 12: DPI Stimulates Sustained Increase in Glycolytic Activity Through Nuclear Translocation of PKM2 and Transcriptional Activation

How does DPI stimulate transcription of genes in the glycolysis pathway? PKM2 is known to be present in monomeric, dimeric and tetrameric forms. While the tetrameric form exhibits glycolytic enzymatic activity, the dimeric form can translocate into the nucleus and function as a transcriptional cofactor to activate expression of c-Myc, which, in turn, can directly activate the transcription of almost all glycolytic genes through binding the classical E-box sequence. To test this mechanism, we first determined if PKM2 is required for DPI-induced transcription of glycolytic genes. BMDMs were prepared from wild-type and Pkm^{-/-} mice, incubated with or without 50 and 500 nM DPI for 24 hours, and the transcript levels of key glycolytic genes were quantified by RT-PCR. In a dose-dependent manner, DPI stimulated the transcription of Pkm, Ldha and Hk2 in the wild-type but not in Pkm^{-/-} BMDMs (FIG. 23A), suggesting PKM2 is required for mediating DPI-stimulated transcription of glycolytic genes.

Next, we determined if DPI induces formation of dimeric PKM2 and nuclear translocation. ImKCs were treated with 50 or 500 nM DPI for 6 or 12 hours, lysed and analyzed directly by Native PAGE gel, followed by anti-PKM2 Western blotting. While PKM2 was found in monomeric and tetrameric forms without DPI treatment, dimeric form was induced following DPI treatment in a dose-dependent manner (FIG. 23B). Induction of dimeric PKM2 by DPI was further confirmed by DSS crosslinking followed by Western blotting and abolished by inhibition of ERK1/2 with SCH772984 (FIGS. 30A-30B), consistent with the previous reports. To further determine PKM2 nuclear translocation following DPI treatment, both ImKCs and human primary KCs were not treated or treated with DPI for 24 hours and then stained with anti-PKM2. Without DPI treatment, anti-PKM2 fluorescent signals were localized in the cytosol, whereas with DPI treatment, significant amount of anti-PKM2 fluorescent signals was detected in the nucleus (FIG. 23C), suggesting translocation of PKM2 from cytosol into the nucleus following DPI treatment.

We also determined if c-Myc is induced by DPI in a PKM2-dependent manner. As shown in FIG. 23A, in a dose-dependent manner, DPI stimulated the transcription of c-Myc in wild-type but not Pkm^{-/-} BMDMs. To determine whether DPI activates c-Myc transcriptional activity, we performed c-Myc luciferase reporter assays in the parental ImKCs and Pkm^{-/-} ImKCs. Luciferase activity was induced by DPI only in parental ImKCs not in Pkm^{-/-} ImKCs (FIG. 23D), showing that DPI activates c-Myc transcriptional activity in PKM2-dependent manner.

Taken together, these results show that DPI stimulates sustained increase in glycolytic activity through nuclear translocation of PKM2, transcriptional activation of c-Myc, and transcription of glycolytic genes.

### Example 13: DPI Inhibits HFD-Induced Obesity and Liver Pathogenesis Through PKM2 Expression in Kupffer Cells

To explore the in vivo consequence of DPI on glycolysis, we examined fast glucose response in DPI pretreated mice. C57BL/6 (B6) mice were injected intraperitoneally (i.p.) with 2 mg/kg DPI and 6 hours later mice were injected i.p. with 1.5 mg/kg glucose. Blood glucose levels were measured before DPI injection, 6 hours after DPI injection and at different time points after glucose injection. As shown in FIG. 31, mice had the same levels of blood glucose before DPI injection. 6 hours after DPI injection, DPI treated mice had a significantly lower level of blood glucose and maintained significantly lower levels of glucose 15 and 30 min after glucose injection, suggesting DPI stimulates an increased metabolic rate of blood glucose. We further examined whether DPI inhibits high fat diet (HFD) induced obesity and liver pathogenesis. B6 mice at 5 weeks of age were fed with HFD for a total of 8 weeks. Three weeks after the start on HFD when mice had exhibited significant weight gain, a portion of the mice were given vehicle (PEG3000) and the rest of the mice were given DPI (2 mg/kg) in vehicle i.p. every five days. Among the HFD-fed mice, DPI treatment immediately and significantly reduced the weight gain as compared to vehicle treated group (FIG. 24A) without affecting the weekly food intake (FIG. 24B). Consistently, DPI-treated mice had significantly lower levels of iWAT after 8 weeks on HFD (FIG. 24C). Notably, DPI-treated HFD mice gained weight at similar rate as mice fed with normal diet (ND) (FIG. 24A), suggesting that DPI inhibits weight gain due to extra fat uptake but not the normal growth. Glucose tolerance test showed that the DPI-treated HFD mice displayed a significant increase in glucose tolerance compared to the vehicle-treated HFD mice (FIG. 24D). DPI treatment also significantly reduced the lipid deposition in the liver as compared to vehicle-treated HFD mice (FIG. 23E). Consistently, the concentrations of serum ALT and AST in HFD-fed mice were significantly higher than in normal diet-fed mice (FIG. 23F). DPI administration significantly reduced the HFD-induced elevation of serum AST and ALT.

We also examined the effect of DPI on hepatic steatosis. B6 mice were fed with HFD for 16 weeks. Nine weeks after HFD when mice became obese, DPI (2 mg/kg) was given once every 5 days for a total of 10 doses. DPI also significantly reduced the weight gain without affecting the weekly food intake (FIGS. 24A-24B). The weight of iWAT was significantly lower in DPI-treated group than in vehicle-treated group (FIG. 31C). Similarly, the DPI-treated HFD mice displayed an increased glucose tolerance and had reduced lipid droplet, steatosis and collagen deposition in the liver (FIGS. 31D-31E). Together, these results show that DPI inhibits HFD-induced obesity, lipid deposition and hepatic steatosis in mice.

To investigate the cell types in the liver that mediate DPI's effect, we analyzed the expression of PKM2 in different cell types in the livers using known single cell RNAseq data. In both human and mice, PKM2 was highly expressed in Kupffer cells and intermediately expressed in other immune cells, while PKM1 (PKLR) was exclusively expressed in APOC3′ hepatocytes (FIG. 25). To directly test whether PKM2 expression in Kupffer cells mediate the effect of DPI, we constructed KC-specific PKM2 knockout (Pkm^{-/-}) mice by crossing Clec4f-Cre mice with PKM2 floxed (Pkm^{f/f}) mice. KC-specific Pkm^{-/-} mice were fed with HFD for 8 weeks starting at 5 weeks of age. Three weeks after HFD, half of the mice were given vehicle and the other half was given DPI (2 mg/kg) i.p. every 5 days. As shown in FIGS. 24I-24J, DPI did not reduce the HFD-induced body-weight gain and lipid droplet deposition in the livers of KC-specific Pkm^{-/-} mice. Similarly, KC-specific Pkm^{-/-} mice with or without DPI treatment had similar glucose tolerance, serum AST and ALT levels, except that DPI treated mice has a significantly lower level of iWAT (FIG. 33). These results show that DPI inhibits HFD-induced obesity and liver pathogenesis is dependent on PKM2 expression in Kupffer cells.

### Example 14: DPI Upregulates Glycolysis and Suppresses Inflammatory Responses of Kupffer Cells in HFD-Fed Mice

To further investigate the effects of DPI on Kupffer cells in vivo, we purified KCs from vehicle- or DPI-treated HFD-fed mice and age-matched mice on the normal diet, and performed RNA-seq. GSEA and functional enrichment analysis showed that upregulation of genes associated with immune and inflammatory responses in KCs from mice fed with HFD or ND (FIGS. 25A-25C). Expression of genes involved in inflammation were significantly suppressed in KCs from HFD mice following DPI treatment. In contrast, expression of many other genes that was down-regulated in KCs from HFD mice were significantly upregulated after DPI treatment (FIG. 25A). Interestingly, expression of genes involved in glycolysis, oxidative phosphorylation and fatty acid metabolism was downregulated in KCs of HFD mice, whereas expression of these genes was upregulated in KCs from HFD mice after DPI treatment (FIGS. 25A-25C). Macrophage polarization index (MPI) analysis showed that KCs were polarized to M1 in HFD-fed mice but to M2 in mice on normal diet, while KCs were reprogrammed to an intermediated phenotype in DPI-treated HFD mice (FIG. 25D). These results suggest that DPI upregulates glycolysis and suppresses inflammatory responses of KCs in HFD-fed mice.

### Example 15: DPI Upregulates Glycolysis and Suppresses Inflammatory Responses of Kupffer Cells from Patients with NAFLD

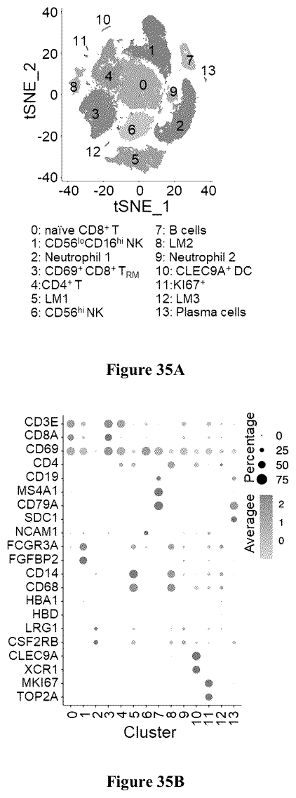

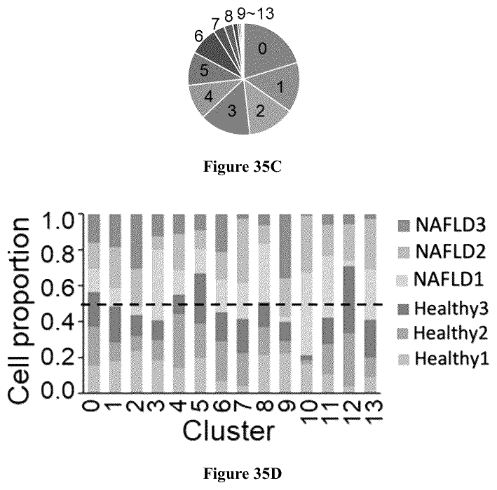

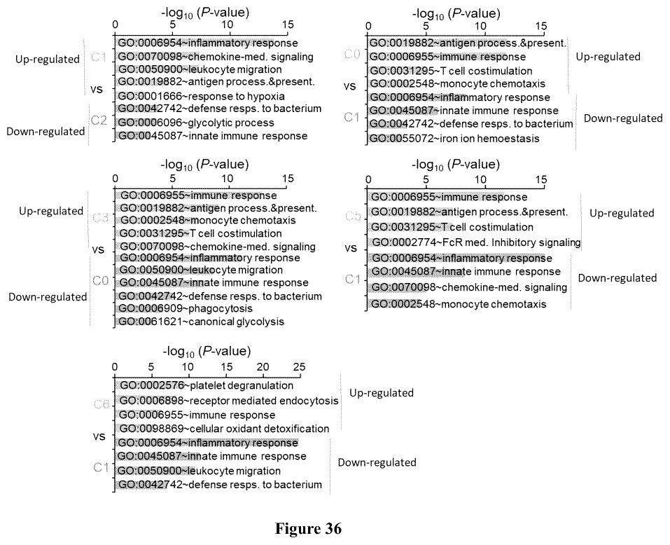

Single cell RNAseq analysis of liver cells from NASH and cirrhosis patients has identified TREM2⁺ disease-associated macrophages (DAMs) in the liver that have lower expression of metabolic genes. To determine whether the DAMs are also present in patients with NFALD, we performed scRNAseq of immune cells from liver biopsies of 3 healthy donors and 3 NFALD patients. Fourteen cell clusters were identified, including naïve CD8⁺ T cells, resident memory CD8⁺ (T_{RM}) cells, CD4⁺ T cells, B and plasma cells, CD56^{low} and CD56^{hi} NK cells, macrophages or KCs, neutrophils and proliferating cells (FIG. 35). Three liver macrophage populations (LM1, LM2, LM3) were identified and further analyzed. As shown in FIGS. 26A-26E, LMs were reclassified into 7 clusters, which could be annotated. Cluster 1 (C1) and C2 were resident KCs as they expressed MNDA and FCN1. C1 differed from C2 by expressing higher levels of inflammatory genes (FIG. 36) whereas C2 expressed higher levels of glycolytic genes, including PGAM1, PKM, GAPDH and ENO1 (FIG. 26C). C0, C3 and C4 all expressed MHC-II (HLA-DRB1, etc.). C4 was like dendritic cells as some cells expressed CD1C. C3 resembled to DAMs by expressing C1QA, APOE, TREM2, CD9, GPNMB and CLEC10A, as well as complement genes (C1QA, etc.). C3 was the only elevated LM population in NFALD, with upregulated pathways of antigen processing and presentation, monocyte chemotaxis, response to wounding and down-regulated pathways of immune response, glycolysis, phagocytosis (FIG. 26F), as observed in advanced NASH and cirrhosis. Based on the trajectory inference (FIG. 26E) and enriched GO ontology pathways (FIG. 26F and FIG. 36), C0 was likely the intermediate or differentiating LM or KCs between resident KCs (C1 and C2) and DAMs (C3) by co-expressing multiple genes, including CD163, LIPA, CCL3, CCL4 and CXCL3 (FIG. 26C). C5 expressed high levels of myeloid checkpoint receptors LIRB1 and LIRB2. C6 was likely the KCs phagocytosing red blood cells by co-expressing hemoglobin mRNAs (HBD and HBA2) (FIG. 26C and FIG. 36).

To directly examine the effect of DPI on human Kupffer cells from NFALD patients, we purified KCs from two NFALD patients and performed the transcriptional analysis by RNA-seq following DPI treatment ex vivo for 24 hours. The same as human MDMs and mouse ImKCs, the expression of glycolytic genes was upregulated by DPI whereas the expression of DAM markers, including APOE, CLEC10A, TREM2 and C1QA, was downregulated (FIG. 26G). Functional enrichment analysis showed that DPI-treated KCs not only upregulated the expression of glycolytic genes but also suppressed the expression of genes associated with chemokine-mediated signaling, chemotaxis and inflammatory response (FIG. 26H). These results show that DPI also upregulates glycolysis and suppresses inflammatory responses of Kupffer cells from patients with NAFLD.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of identifying a modulator of macrophage activation, comprising:
contacting a primary macrophage cell with a candidate agent;
monitoring or photographing the morphology of the cell contacted with the candidate agent; and
optionally comparing the cell's morphology in the presence of the candidate agent with the cell's morphology in the absence of the candidate agent; wherein a change in morphology in the presence of the candidate agent is indicative of modulation of macrophage activation.

2. The method of claim 1, wherein the primary macrophage cell is a bone marrow-derived macrophage or a monocyte-derived macrophage.

3.-6. (canceled)

7. The method of claim 1, wherein the morphology of the cell is changed from elongated shape to round shape.

8. The method of claim 7, wherein the modulator activates a M1-like macrophage.

9. The method of claim 7, wherein the modulator deactivates a M2-like macrophage.

10. The method of claim 7, wherein the modulator changes a tumor-associated macrophage (TAM) to M1-like macrophage.

11. The method of claim 7, wherein the modulator changes a M2-like macrophage to a M1-like macrophage.

12. The method of claim 7, wherein the modulator changes a M-CSF macrophage to a M1-like macrophage.

13. The method of claim 7, wherein the modulator changes a GM-CSF macrophage to a M1-like macrophage.

14. The method of claim 7, wherein the modulator changes a primary macrophage to a M1-like macrophage.

15. The method of claim 7, wherein the modulator induces LPS, IFNγ or TNFα.

16. The method of claim 7, wherein the modulator activates a serotonin transporter or receptor, a histamine transporter or receptor, a dopamine transporter or receptor, an adrenoceptor, VEGF, EGF and/or leptin.

17. The method of claim 7, wherein the modulator is a M1-activating compound.

18. The method of claim 7, wherein the modulator is cytochalasin-B, fenbendazole, parbendazole, methiazole, alprostadil, FTY720, penfluridol, taxol, smer-3, cantharidin, SCH79797, mitoxantrone, niclosamide, MS275, HMN-214, DPI, thiostrepton, evodiamine, cucurbitacin-I, NVP 231, Chlorhexidine, Diphenyleneiodonium, LE135, Fluvoxamine, Mocetinostat, Pimozide, NP-010176, Celastrol, FTY720, WP1130, Prulifloxacin, dihydrocelastryl diacetate, or Quinolinium.

19. The method of claim 8, wherein the M1-like macrophage mediates a pro-inflammatory response, an anti-microbial response, and/or an anti-tumor response.

20.-23. (canceled)

24. The method of claim 1, wherein the morphology of the cell is changed from round shape to elongated shape.

25. The method of claim 24, wherein the modulator activates a M2-like macrophage.

26.-33. (canceled)

34. The method of claim 24, wherein the modulator is Bostunib, Su11274, Alsterpaullone, Alrestatin, Bisantrene, triptolide, lovastatin, QS 11, Regorafenib, Sorafenib, MLN2238, GW-843682X, KW 2449, Axitinib, JTE 013, Purmorphamine, Arcyriaflavin A, Dasatinib, NVP-LDE225, 1-Naphthyl PP1, Selamectin, MGCD-265, podofilox, colchicine, or vinblastine sulfate.

35.-38. (canceled)

39. A method of treating cancer, fibrosis, or an infectious disease, comprising administering to a subject in need thereof an effective amount of a modulator of macrophage activation; wherein the modulator changes the morphology of a macrophage cell from elongated shape to round shape or the modulator activates a serotonin transporter or receptor, a histamine transporter or receptor, a dopamine transporter or receptor, an adrenoceptor, VEGF, EGF and/or leptin, or the modulator is KW 2449.

40.-62. (canceled)

63. A method of treating an inflammatory disease, a metabolic disease, an autoimmune disease, or a neurodegenerative disease, comprising administering to a subject in need thereof an effective amount of a modulator of macrophage activation; wherein the modulator changes the morphology of a macrophage cell from round shape to elongated shape, the modulator inhibits a serotonin transporter or receptor, a histamine transporter or receptor, a dopamine transporter or receptor, an adrenoceptor, VEGF, EGF and/or leptin, or the modulator is KW 2449.

64.-96. (canceled)


SEQUENCE LISTING (40 sequences)

SEQ ID NO: 1 - DNA, 22 bp, synthetic construct
  source 1..22: mol_type = other DNA
cattggcttgcgagacgtagac

SEQ ID NO: 2 - DNA, 22 bp, synthetic construct
  source 1..22: mol_type = other DNA
gctgaaggtctcttccatcacc

SEQ ID NO: 3 - DNA, 23 bp, synthetic construct
  source 1..23: mol_type = other DNA
caaggaacttcttgccaatccag

SEQ ID NO: 4 - DNA, 22 bp, synthetic construct
  source 1..22: mol_type = other DNA
ccaagatccacaggcaaagcca

SEQ ID NO: 5 - DNA, 22 bp, synthetic construct
  source 1..22: mol_type = other DNA
ctgctgtaacgatgaagccctg

SEQ ID NO: 6 - DNA, 22 bp, synthetic construct
  source 1..22: mol_type = other DNA
gctgtaggaagctcatctctcc

SEQ ID NO: 7 - DNA, 22 bp, synthetic construct
  source 1..22: mol_type = other DNA
tactcacttccacaggagcagg

SEQ ID NO: 8 - DNA, 22 bp, synthetic construct
  source 1..22: mol_type = other DNA
ctccagtgtagccatccttagg

SEQ ID NO: 9 - DNA, 22 bp, synthetic construct
  source 1..22: mol_type = other DNA
tgatacgcctgagtggctgtct

SEQ ID NO: 10 - DNA, 22 bp, synthetic construct
  source 1..22: mol_type = other DNA
cacaagagcagtgagcgctgaa

SEQ ID NO: 11 - DNA, 22 bp, synthetic construct
  source 1..22: mol_type = other DNA
ggtgcctatgtctcagcctctt

SEQ ID NO: 12 - DNA, 23 bp, synthetic construct
  source 1..23: mol_type = other DNA
gccatagaactgatgagagggag

SEQ ID NO: 13 - DNA, 22 bp, synthetic construct
  source 1..22: mol_type = other DNA
acggctgagtttcagtgagacc

SEQ ID NO: 14 - DNA, 22 bp, synthetic construct
  source 1..22: mol_type = other DNA
cactctggtaggtgtaaggtgc

SEQ ID NO: 15 - DNA, 22 bp, synthetic construct
  source 1..22: mol_type = other DNA
gctacaagaggatcaccagcag

SEQ ID NO: 16 - DNA, 22 bp, synthetic construct
  source 1..22: mol_type = other DNA
gtctggacccattccttcttgg

SEQ ID NO: 17 - DNA, 22 bp, synthetic construct
  source 1..22: mol_type = other DNA
cctgctgctttgcctacctctc

SEQ ID NO: 18 - DNA, 22 bp, synthetic construct
  source 1..22: mol_type = other DNA
acacacttggcggttccttcga

SEQ ID NO: 19 - DNA, 22 bp, synthetic construct
  source 1..22: mol_type = other DNA
catccagagcttgagtgtgacg

SEQ ID NO: 20 - DNA, 22 bp, synthetic construct
  source 1..22: mol_type = other DNA
ggcttcagggtcaaggcaaact

SEQ ID NO: 21 - DNA, 20 bp, synthetic construct
  source 1..20: mol_type = other DNA
agtatgactccactcacggc

SEQ ID NO: 22 - DNA, 20 bp, synthetic construct
  source 1..20: mol_type = other DNA
gttcacacccatcacaaaca

SEQ ID NO: 23 - DNA, 22 bp, synthetic construct
  source 1..22: mol_type = other DNA
gagacagggaagtctgaagcac

SEQ ID NO: 24 - DNA, 22 bp, synthetic construct
  source 1..22: mol_type = other DNA
ccagcagtagttgctcctcttc

SEQ ID NO: 25 - DNA, 22 bp, synthetic construct
  source 1..22: mol_type = other DNA
gtctcctctgacttcaacagcg

SEQ ID NO: 26 - DNA, 22 bp, synthetic construct
  source 1..22: mol_type = other DNA
accaccctgttgctgtagccaa

SEQ ID NO: 27 - DNA, 22 bp, synthetic construct
  source 1..22: mol_type = other DNA
ctcttctgcctgctgcactttg

SEQ ID NO: 28 - DNA, 22 bp, synthetic construct
  source 1..22: mol_type = other DNA
atgggctacaggcttgtcactc

SEQ ID NO: 29 - DNA, 22 bp, synthetic construct
  source 1..22: mol_type = other DNA
ccacagaccttccaggagaatg

SEQ ID NO: 30 - DNA, 23 bp, synthetic construct
  source 1..23: mol_type = other DNA
gtgcagttcagtgatcgtacagg

SEQ ID NO: 31 - DNA, 22 bp, synthetic construct
  source 1..22: mol_type = other DNA
ggcagaaagcttgtctcaaccc

SEQ ID NO: 32 - DNA, 22 bp, synthetic construct
  source 1..22: mol_type = other DNA
ctccttcaggaacagccaccaa

SEQ ID NO: 33 - DNA, 22 bp, synthetic construct
  source 1..22: mol_type = other DNA
tctccgagatgccttcagcaga

SEQ ID NO: 34 - DNA, 22 bp, synthetic construct
  source 1..22: mol_type = other DNA
tcagacaaggcttggcaaccca

SEQ ID NO: 35 - DNA, 21 bp, synthetic construct
  source 1..21: mol_type = other DNA
tcattccctgatgttacgagc

SEQ ID NO: 36 - DNA, 21 bp, synthetic construct
  source 1..21: mol_type = other DNA
tcttccctctccattgtgttg

SEQ ID NO: 37 - DNA, 22 bp, synthetic construct
  source 1..22: mol_type = other DNA
gtgtgatgactcttgggacttg

SEQ ID NO: 38 - DNA, 20 bp, synthetic construct
  source 1..20: mol_type = other DNA
aggatgactgacgggatgag

SEQ ID NO: 39 - DNA, 22 bp, synthetic construct
  source 1..22: mol_type = other DNA
gactgataagtggagggtgagg

SEQ ID NO: 40 - DNA, 19 bp, synthetic construct
  source 1..19: mol_type = other DNA
ccagagaggaacccattcg